(19) United States
(12) Patent Application Publication
(10) Pub. No.: US 2009/0186050 A1
(43) Pub. Date: Jul. 23, 2009

(54) Live attenuated metapneumovirus strains and their use in vaccine formulations and chimeric metapneumovirus strains

(76) Inventors: Ron A.M. Fouchier, Rotterdam (NL); Sander Herfst, Capelle aan den Ijssel (NL); Miranda de Graaf, Amsterdam (NL)

Correspondence Address: JONES DAY, 222 EAST 41ST ST, NEW YORK, NY, 10017, US

(21) Appl. No.: 12/292,000

(22) Filed: Nov. 14, 2008

Related U.S. Application Data
(60) Provisional application No. 61/003,562, filed on Nov. 16, 2007.

(51) Int. Cl. A61K 39/155; C12N 7/01; A61P 31/12

(52) U.S. Cl. 424/211.1; 435/235.1

(57) ABSTRACT

The invention relates to an isolated mammalian negative strand RNA virus, metapneumovirus (MPV), within the sub-family Pneumoviridae, of the family Paramyxoviridae with one or more genetic modifications. The present invention also relates to the mutant components, i.e., nucleic acids and proteins, of these mutant mammalian MPVs. These mutant mMPV can be attenuated. These mutant mMPVs can encode non-native sequences. The invention further relates to vaccine formulations comprising the mMPV, including recombinant and chimeric forms of said viruses. The vaccine preparations of the invention encompass multivalent vaccines, including bivalent and trivalent vaccine preparations. In addition, the invention relates to chimeric viral RNA polymerase complex and assays using these chimeric RNA polymerase complexes. The chimeric RNA polymerase complexes of the invention are composed of different RNA polymerase components from different viruses of the family of paramyxoviridae.

This application claims and is entitled to priority benefit of U.S. provisional application Ser. No. 61/003,562, filed Nov. 16, 2007, which is incorporated herein by reference in its entirety.

### 1. INTRODUCTION

The invention relates to an isolated mammalian negative strand RNA virus, metapneumovirus (MPV), within the sub-family Pneumoviridae, of the family Paramyxoviridae with one or more genetic modifications. The present invention also relates to the mutant components, i.e., nucleic acids and proteins, of these mutant mammalian MPVs. These mutant mMPV can be attenuated. These mutant mMPVs can encode non-native sequences. The invention further relates to vaccine formulations comprising the mMPV, including recombinant and chimeric forms of said viruses. The vaccine preparations of the invention encompass multivalent vaccines, including bivalent and trivalent vaccine preparations. In addition, the invention relates to chimeric viral RNA polymerase complex and assays using these chimeric RNA polymerase complexes. The chimeric RNA polymerase complexes of the invention are composed of different RNA polymerase components from different viruses of the family of paramyxoviridae.

### 2. BACKGROUND OF THE INVENTION

The human metapneumovirus (hMPV) was first isolated from respiratory specimens obtained from children hospitalized for acute respiratory tract illness (RTI) in The Netherlands (van den Hoogen et al., 2001, Nat Med 7:719-24). Clinical manifestations of hMPV infections are similar to those caused by respiratory syncytial virus (RSV), ranging from mild respiratory illness to bronchiolitis and pneumonia (van den Hoogen, et al., 2003, J Infect Dis 188; Williams et al., 2006, J Infect Dis 193:387-95). Two surface glycoproteins, the attachment protein (G) and the short-hydrophobic protein (SH), are highly variable among virus isolates, the fusion protein (F) is highly conserved, and antibodies induced against F correlate with protection in animal models (Skiadopoulos et al., 2006, Virology 345:492-501; Tang et al., 2005, Vaccine 23:1657-67).

Several vaccination strategies have been explored since the discovery of hMPV, including subunit vaccines (Cseke et al., 2007, 81:698-707; Herfst et al., 2007, J Gen Virol, in press), live attenuated vaccines (LAVs) (Biacchesi et al., 2005, J Virol 79:12608-13; Pham et al., 2005, J Virol 79:15114-22; and Tang et al., 2005, Vaccine 23:1657-67) and formalin-inactivated (FI-) hMPV. The upper respiratory tract (URT) of cotton rats immunized with FI-hMPV were almost completely protected against infection, but an increase in lung pathology combined with a change in cytokine profiles was observed (Yim et al., 2007, Vaccine 25:5034-40). This observation may indicate that the enhanced disease observed in RSV-infected children upon immunization with FI-RSV (Kim et al., 1969, Am J Epidemiol 89:422-34) may also be a problem if such vaccines are applied for hMPV. Human metapneumovirus (hMPV) is an enveloped, non-segmented, negative-strand RNA virus that causes respiratory tract illnesses primarily in infants, young children, frail elderly and immunocompromised individuals (Crowe, 2004, Pediatr. Infect. Dis. 23, S215-221; Falsey et al., 2003, J. Infect. Dis. 187, 785-790; Kahn, 2006, Clin. Microbiol. Rev. 19, 546-557; Pelletier et al., 2002, Emerg. Infect. Dis. 8, 976-978; van den Hoogen et al., 2001, Nat. Med. 7, 719-724; van den Hoogen et al., 2003, J. Infect. Dis. 188, 1571-1577). HMPV is a member of the Paramyxoviridae family, subfamily Pneumovirinae, genus Metapneumovirus, and can be divided into two main genetic lineages (A and B) each consisting of two sublineages (A1, A2, B1 and B2) (van den Hoogen et al., 2004, Emerg. Infect. Dis. 10, 658-666). The only other identified member of the Metapneumovirus genus is the avian metapneumovirus (aMPV). AMPV has been found to infect domestic poultry worldwide, causing acute respiratory infections (Cook, 2000, Rev. Sci. Tech. 19, 602-613). AMPVs have been classified into four subgroups, A through D (Bayon-Auboyer et al., 1999, Arch. Virol. 144, 1091-1109; Eterradossi et al., 1995, Zentralbl. Veterinarmed. B. 42, 175-186; Juhasz & Easton, 1994, J. Gen. Virol. 75, 2873-2880; Seal, 1998, Virus Res. 58, 45-52). AMPV-C was first detected in the United States and is more closely related to hMPV than the other aMPV subgroups (Govindarajan & Samal, 2004, Virus Res. 105, 59-66; Govindarajan & Samal, 2005, Virus Genes 30, 331-333; Govindarajan et al., 2004, J. Gen. Virol. 85, 3671-3675; Toquin et al., 2003, J. Gen. Virol. 84, 2168-2178; van den Hoogen et al., 2002, Virology 295, 119-132; Yunus et al., 2003, Virus Res. 93, 91-97). The only other known member of the Pneumovirinae subfamily that infects humans is the human respiratory syncytial virus (hRSV). In comparison to hRSV, metapneumoviruses lack the non-structural proteins NS1 and NS2 and the order of the genes between the matrix (M) and large polymerase (L) genes is different; hMPV/aMPV, ‘3 le-N-P-M-F-M2-5H-G-L-tr 5’, RSV-A2, ′3 le-NS1-NS2-N-P-M-5H-G-F-M2-L-tr 5′.

The viral genome of all members of the Pneumovirinae subfamily is of antisense polarity and assembled in a ribonucleoprotein complex (RNP). This RNP contains the viral genomic RNA (vRNA) encapsidated by the nucleocapsid protein (N), the phosphoprotein (P) and the L protein. In analogy to other paramyxoviruses, the L protein is responsible for the main catalytic activities of the viral polymerase complex (Grdzelishvili et al., 2005, J. Virol. 79, 7327-7337; Hercyk et al., 1988, Virology 163, 222-225; Ogino et al., 2005, J. Biol. Chem. 280, 4429-4435). The assembly and polymerase cofactor P and the L protein form the minimal complex needed for viral polymerase activity (Mazumder & Barik, 1994, Virology 205, 104-111). RSV RNA synthesis involves an additional viral protein, the M2.1 protein, a transcriptional elongation factor that enhances the synthesis of readthrough mRNAs (Collins et al., 1996, Proc. Natl. Acad. Sci. USA 93, 81-85; Feams & Collins, 1999, J. Virol. 73, 5852-5864; Hardy & Wertz, 1998, J. Virol. 72, 520-526). For hMPV the function of M2.1 is not completely understood as recombinant hMPV can be recovered in the absence of M2.1 and viruses from which the M2.1 gene is deleted grow efficiently in vitro but not in vivo (Buchholz et al., 2005, J. Virol. 79, 6588-6597; Herfst et al., 2004, J. Virol. 78, 8264-8270). The 3′(leader) and 5′(trailer) ends contain the viral promoters necessary for replication and transcription. Transcription of paramyxoviruses is further directed by gene start (GS) and gene end (GE) sequences flanking each of the open reading frames (ORFs) in the viral genome. Transcription of the viral genome results in a gradient of transcripts, steadily decreasing toward the 5′ end of the genome. Thus, the gene order roughly reflects the relative amount of gene products required for efficient virus replication.

Several vaccination strategies have been explored since the discovery of hMPV, including subunit vaccines (Cseke et al., 2007, J. Virol. 81, 698-707; Herfst et al., 2007, J. Gen. Virol. In press), live attenuated vaccines (“LAVs”; Biacchesi et al., 2005, J. Virol. 79, 12608-12613; Pham et al., 2005, J. Virol. 79, 15114-15122; Tang et al., 2005, Vaccine 23, 1657-1667) and formalin-inactivated (FI-) hMPV. The upper respiratory tract (URT) of cotton rats immunized with FI-hMPV were almost completely protected against infection, but an increase in lung pathology combined with a change in cytokine profiles was observed (Yim et al. 2007, Vaccine 25, 5034-5040). This observation may indicate that the enhanced disease observed in RSV-infected children upon immunization with FI-RSV (Kim et al., Am. J. Epidemiol. 89, 422-434) may also be a problem if such FI-vaccines are applied for hMPV. For LAVs, no enhanced disease has been observed in studies performed in naïve animals with RSV and hMPV. LAVs may be useful to prime or boost hMPV-specific immune responses, since such viruses have the advantage of mimicking a natural infection, and thus could provide protection against subsequent infections without inducing enhanced disease. Recently developed reverse genetics systems for hMPV (Biacchesi et al., 2004, Virology 321, 247-259; Herfst et al., 2004, J. Virol. 78, 8264-8270) facilitate the modification of viral genomes and thus provide a powerful tool to design LAVs.

hMPV deletion mutants, chimeric viruses based on hMPV and avian metapneumovirus (aMPV), and a human/bovine parainfluenza virus type 3 (b/hPIV3) expressing the F protein of hMPV (Biacchesi et al., 2005, J. Virol. 79, 12608-12613; Pham et al., 2005, J. Virol. 79, 15114-15122; Tang et al., 2005, Vaccine 23, 1657-1667) have recently been described.

Citation or discussion of a reference herein shall not be construed as an admission that such is prior art to the present invention.

### 3. SUMMARY OF THE INVENTION

The invention relates to mutants of mammalian metapneumovirus (mMPV). In certain aspects of the invention, the mammalian metapneumovirus is a human metapneumovirus (hMPV). The mammalian MPV can be a variant A1, A2, B1 or B2 mammalian MPV. In certain embodiments, the mutant mMPV or hMPV is attenuated and can be used as a vaccine. In certain embodiments, the mutant mMPV or hMPV of the invention can be used in an immunogenic composition. In certain embodiments, the mutant mMPV or hMPV is temperature-sensitive.

The invention also relates to an assay system to test the activity of a chimeric viral RNA polymerase complex that is composed of RNA polymerase subunits from different paramyxoviruses.

In certain embodiments, the invention provides for isolated mammalian MPV comprising genetic modifications. In one aspect of this embodiment, an isolated mammalian MPV is provided which comprises a genetic modification resulting in an amino acid substitution, deletion, or insertion at one or more amino acid positions selected from the group consisting of: position 66 in the P protein; positions 9, 38, 52, and 132 in the M protein; positions 93, 101, 280, 471, 532, and 538 in the F protein; position 187 in the M2 protein; positions 139 and 164 in the G protein; and positions 235, 323, and 1453 in the L protein, with the proviso that the modification at position 101 in the F protein is not a substitution to Proline and that the modification at position 93 in the F protein is not a substitution to Lysine. In a more specific embodiment, the genetic modification results in an amino acid substitution, deletion, or insertion at one or more amino acid positions selected from the group consisting of: position 66 in the P protein; position 132 in the M protein; positions 101, 280, and 471 in the F protein; position 187 in the M2 protein; position 139 in the G protein; and positions 235, 323, and 1453 in the L protein, with the proviso that modification at position 101 in the F protein is not a substitution to Proline. In another specific embodiment, the genetic modification results in an amino acid substitution, deletion, or insertion at one or more amino acid positions selected from the group consisting of: position 132 in the M protein; positions 101, 280, and 471 in the F protein; position 187 in the M2 protein; position 139 in the G protein; and position 1453 in the L protein, with the proviso that modification at position 101 in the F protein is not a substitution to Proline. In a further embodiment, the genetic modification results in an amino acid substitution, deletion, or insertion at one or more amino acid positions selected from the group consisting of: positions 235 and 323 in the L protein. In a specific embodiment, the isolated mammalian metapneumovirus comprises genetic modifications resulting in amino acid substitution, deletion, or insertion at amino acid positions 235 and 323 in the L protein.

In another embodiment, the invention provides an isolated mammalian MPV comprising a genetic modification at one or more of the nucleotide positions selected from the group consisting of: position 197 in the P open reading frame; position 25, 113, 155, 336, 394, and 436 in the M open reading frame; positions 277, 301, 839, 1412, 1594, and 1612 in the F open reading frame; position 560 in the M2 open reading frame; position 415 and 491 in the G open reading frame; and positions 703, 967, and 4357 in the L open reading frame.

In certain embodiments, the isolated mammalian MPV of the invention further comprises a genetic modification that is a silent mutation, i.e., it does not result in an amino acid exchange. In more specific embodiments, the isolated mammalian MPV comprises a silent mutation at one or more of the nucleotide positions selected from the group consisting of: positions 336 and 436 in the M open reading frame.

In certain embodiments, the isolated mammalian MPV of the invention further comprises a genetic alteration that results in an amino acid exchange at amino acid 109 of the F protein. In a more specific embodiment, the isolated mammalian MPV of the invention further comprises a mutation at nucleotide position 325 of the F protein that results in a serine at that position. In a more specific embodiment, the isolated mammalian MPV of the invention further comprises a mutation at nucleotide position 325 of the F protein.

In another embodiment, the invention provides an isolated mammalian MPV comprising a genetic modification at one or more of the nucleotide positions selected from the group consisting of: position 197 in the P open reading frame; position 25, 113, 155, 336, 394, and 436 in the M open reading frame; positions 277, 301, 839, 1412, 1594, and 1612 in the F open reading frame; position 560 in the M2 open reading frame; position 415 and 491 in the G open reading frame; and positions 703, 967, and 4357 in the L open reading frame, wherein the genetic modifications at positions 336 and 436 in the M open reading frame result in silent mutations.

In a further embodiment, the invention provides an isolated mammalian MPV comprising a genetic modification resulting in one or more amino acid changes selected from the group consisting of: (i) position 66 in the P gene is altered to Val; (ii) position 9 in the M gene is altered to His; (iii) position 38 in the M gene is altered to Ser; (iv) position 52 in the M gene is altered to Pro; (v) position 132 in the M gene is altered to Pro; (vi) position 93 in the F gene is altered to Lys; (vii) position 280 in the F gene is altered to Gly; (viii) position 471 in the F gene is altered to Arg; (ix) position 532 in the F gene is altered to Tyr; (x) position 538 in the F gene is altered to Tyr; (xi) position 187 in the M2 gene is altered to Ile; (xii) position 139 in the G gene is altered to Pro; (xiii) position 164 in the G gene is altered to Pro; (xiv) position 235 in the L gene is altered to Arg; (xv) position 323 in the L gene is altered to Asp; and (xvi) position 1453 in the L gene is altered to Leu. In one aspect of this embodiment, the amino acid changes represented in (i) to (xvi) are combined with genetic modifications at one or more of the nucleotide positions selected from the group consisting of: positions 336 and 436 in the M open reading frame, wherein the genetic modifications at positions 336 and 436 in the M open reading frame result in silent mutations.

In embodiments of the invention wherein isolated mammalian MPV comprising several potential amino acid modifications are provided, the isolated mammalian MPV may have at least two, at least three, at least four, at least five, at least six, at least seven or at least eight of the specified genetic modifications.

In another embodiment, the invention provides for a recombinant mammalian MPV comprising two or more genetic modifications, wherein the genetic modification is an amino acid substitution, deletion, or insertion amino acid position 456 of the L gene; position 1094 of the L gene; or position 1246 of the L gene; or a nucleotide substitution, deletion, or insertion at the gene start sequence of the M2 gene.

In yet another embodiment, the invention provides for a recombinant mammalian MPV, comprising an alteration in the gene start sequence of the M2 gene; an alteration in the L gene such that Phe at amino acid position 456 is mutated to Leu; and an alteration of the L gene such that Met at amino acid position 1094 is mutated to Val.

In certain embodiments of the invention the mutant isolated mammalian MPV carries an amino acid exchange that is encoded by two or three nucleotide substitutions per codon, i.e., a stabilized codon.

In embodiments of the invention comprising isolated mammalian MPV, the isolated mammalian MPV may be temperature-sensitive. In certain embodiments, the isolated mammalian MPV may be a human MPV. In more specific embodiments, the isolated mammalian MPV may be hMPV variant A1, A2, B1, or B2. In other specific embodiments, the isolated mammalian MPV may be hMPV strain NL/1/99, NL/17/00, NL/1/00, or NL/1/94.

In another embodiment of the invention, a method is provided for stimulating the immune response against mammalian MPV in a mammal comprising administering to the mammal an isolated mammalian MPV of the invention including, but not limited to, the isolated mammalian MPV comprising the genetic modifications described above. In one aspect of this embodiment, the mammal is a human. In another aspect of this embodiment, the isolated mammalian MPV is a human MPV, wherein the hMPV can in some aspects be hMPV variant A1, A2, B1, or B2. In other aspects of this embodiment, the hMPV can be hMPV strain NL/1/99, NL/1/00, NL/17/00, or NL/1/94.

The invention also provides for vaccine formulations comprising the isolated mammalian MPV of the invention including, but not limited to, the isolated mammalian MPV comprising the genetic modifications described above, said vaccine formulation to be delivered along with a pharmaceutically acceptable excipient.

In another embodiment of the invention, immunogenic compositions are provided, said immunogenic compositions comprising the isolated mammalian MPV of the invention including, but not limited to, the isolated mammalian MPV comprising the genetic modifications described above, along with a pharmaceutically acceptable excipient.

In another embodiment, the isolated mammalian MPV of the invention including, but not limited to, the isolated mammalian MPV comprising the genetic modifications described above, can be used as a medicament.

In other embodiments, the invention provides a recombinant nucleic acid comprising cDNA encoding the isolated mammalian MPV of the invention, including, but not limited to, the isolated mammalian MPV comprising the genetic modifications described above. In further embodiments, a vector is provided that comprises the recombinant nucleic acid comprising cDNA encoding the isolated mammalian MPV of the invention.

In another embodiment, the invention provides a method of producing a mammalian MPV comprising: a) introducing recombinant nucleic acid comprising cDNA encoding an isolated mammalian MPV of the invention operatively linked to a promoter for DNA-directed RNA polymerase into a host cell, wherein the host cell expresses (i) the N, P, and L proteins of a mammalian MPV and (ii) the DNA-directed RNA polymerase; and b) isolating the virus produced by the cell.

In another embodiment, the invention provides a method for producing a mammalian MPV comprising: a) introducing recombinant nucleic acid comprising cDNA encoding the isolated mammalian MPV of the invention operatively linked to a promoter for a DNA-directed RNA polymerase into a host cell, wherein the host cell expresses the DNA-directed RNA polymerase; b) introducing cDNA encoding the N, P, and L genes of a mammalian metapneumovirus into the host cell; and c) isolating the virus produced by the host cell.

In certain embodiments, the invention provides an infectious chimeric virus, wherein the chimeric virus comprises the genome of a mammalian MPV of a first variant, wherein one or more of the open reading frames in the genome of the mammalian MPV of the first variant have been replaced by the analogous open reading frame from a mammalian MPV of a second variant. In certain embodiments, the invention provides an infectious chimeric virus, wherein the chimeric virus comprises the genome of a mammalian MPV of a first variant, wherein one or more of open reading frames of a mammalian MPV of a second variant are inserted into the genome of the mammalian MPV of the first variant.

In certain embodiments, the invention provides an infectious chimeric virus, wherein the chimeric virus comprises the genome of a mammalian MPV, wherein one or more of the open reading frames in the genome of the mammalian MPV have been replaced by an ORF which encodes one or more of (i) an avian MPV F protein; (ii) an avian MPV G protein (iii) an avian MPV SH protein; (iv) an avian MPV N protein (v) an avian MPV P protein; (vi) an avian MPV M2 protein; (vii) an avian MPV M2.1 protein; (viii) an avian MPV M2.2 protein; or (ix) an avian MPV L protein. In certain embodiments, the invention provides an infectious chimeric virus, wherein the chimeric virus comprises the genome of an avian MPV, wherein one or more of the open reading frames in the genome of the avian MPV have been replaced by an ORF which encodes one or more of (i) a mammalian MPV F protein (ii) a mammalian MPV G protein; (iii) a mammalian MPV SH protein; (iv) a mammalian MPV N protein; (v) a mammalian MPV P protein; (vi) a mammalian MPV M2 protein; (vii) a mammalian MPV M2.1 protein; (viii) a mammalian MPV M2.2 protein; or (ix) a mammalian MPV L protein.

In a certain embodiment, the invention provides a chimeric MPV wherein the N gene of an aMPV replaces the N gene of a hMPV. In another embodiment, the P gene of a hMPV is replaced by the P gene from an aMPV. In still another embodiment, the L gene of a hMPV is replaced with the L gene of an aMPV. In one aspect of these embodiments, the hMPV is serotype B1. In another aspect of these embodiments, the aMPV is from aMPV subgroup C. In still another aspect of these embodiments, the chimeric MPV is attenuated.

In certain embodiments, the invention provides an infectious chimeric or recombinant virus, wherein the chimeric or recombinant virus is rescued using an interspecies or intraspecies polymerase. In one embodiment, the invention provides a chimeric or recombinant virus, wherein the chimeric or recombinant virus is rescued using MPV polymerase. In one embodiment, the invention uses a polymerase from a virus different from the polymerase of the virus to be rescued, i.e., from a different clade, subtype, or other species. In another embodiment, the invention provides an infectious chimeric or recombinant virus, wherein the chimeric or recombinant virus is rescued using the polymerase from another virus, including, but not limited to the polymerase of PIV, AMPV or RSV. By way of example, and not meant to limit the possible combinations, RSV polymerase can be used to rescue MPV; MPV polymerase can be used to rescue RSV; or PIV polymerase can be used to rescue MPV. In yet another embodiment of the invention, the polymerase complex that is used to rescue the recombinant virus is encoded by polymerase proteins from different viruses. By way of example, and not meant to limit the possible combinations, in one embodiment, the polymerase complex proteins are encoded by the N gene of MPV, the L gene of PIV, the P gene of RSV and the M2.1 gene of MPV. In other embodiments, the M2.1 gene is not a component of the polymerase complex. In another embodiment of the invention, and meant by way of example, the polymerase complex proteins are encoded by the N gene of RSV, the L gene of RSV, the P gene of AMPV, and the M2.1 gene of RSV. In another embodiment of the invention, the M2.1 gene is not required to rescue the recombinant virus of the invention. One skilled in the art would be familiar with the types of combinations that can be used to encode the polymerase complex proteins so that the recombinant chimeric virus of the invention is rescued.

In other embodiments, the invention provides an infectious recombinant virus, wherein the recombinant virus is rescued using a chimeric polymerase complex. In a certain aspect of this embodiment, the method comprises the steps of: a) introducing into a host cell cDNA encoding the MPV; b) introducing into the host cell cDNA encoding a chimeric polymerase complex comprising N, P, L, and M2.1 of a MPV, wherein N, P, L, and M2.1 are from at least two different MPV strains; and c) isolating the virus produced by the host cell. In certain aspects of this embodiment, the MPV is a human MPV. In further aspects of this embodiment, the hMPV is variant A1, A2, B1, or B2. In a specific aspect of this embodiment, the hMPV is variant B1. In a certain aspect of this embodiment, the chimeric polymerase complex comprises hMPV B1 and aMPV C, wherein at least one but not all of N, P, L, and M2.1 are from hMPV B1 and at least one but not all of N, P, L, and M2.1 are from aMPV C.

In another embodiment, the invention provides an isolated chimeric viral RNA polymerase complex comprising RNA polymerase complex subunits from at least two different paramyxoviruses. In an aspect of this embodiment, the RNA polymerase complex subunits are the N, P, L, and M2.1 proteins. In another aspect of this embodiment, the two different paramyxoviruses are selected from the group consisting of RSV, PIV, aMPV, and mammalian MPV.

In another embodiment, a method for determining the activity of a chimeric viral RNA polymerase complex is provided, said method comprising the steps: a) introducing into a host cell a cDNA encoding a reporter gene flanked by the genomic termini of a first paramyxoviridae; b) introducing into the host cell cDNAs encoding the RNA polymerase complex subunits from at least two different paramyxoviridae; and c) measuring the activity of the reporter gene. In one aspect of this embodiment, the RNA polymerase complex subunits are heterologous to the first paramyxoviridae. In other aspects of this embodiment, at least one of the RNA polymerase complex subunits is of the first paramyxoviridae. In another aspect of this embodiment, the RNA polymerase complex subunits are the N, P, L, and M2.1 proteins. In another aspect of this embodiment, the first paramyxoviridae is a mammalian MPV. In still another aspect of this embodiment, the at least two different paramyxoviridae whose cDNAs encode the RNA polymerase complex subunits are selected from the group consisting of RSV, PIV, aMPV, and mammalian MPV.

In certain embodiments, the invention provides an immunogenic composition, wherein the immunogenic composition comprises the infectious recombinant or chimeric virus of the invention.

In certain embodiments, the invention provides a pharmaceutical composition, wherein the pharmaceutical composition comprises the infectious recombinant or chimeric virus of the invention.

In certain embodiments, the invention provides a method for detecting a mammalian MPV in a sample, wherein the method comprises amplifying or probing for MPV related nucleic acids, processed products, or derivatives thereof. In a more specific embodiment, the invention provides polymerase chain reaction based methods for the detection of MPV in a sample. In an even further embodiment, the invention provides oligonucleotide probes that can be used to specifically detect the presence of MPV related nucleic acids, processed products, or derivatives thereof. In yet another embodiment, the invention provides diagnostic methods for the detection of MPV antibodies in a host that is infected with the virus.

In certain embodiments, the invention provides a method for treating or preventing a respiratory tract infection in a mammal, said method comprising administering a vaccine comprising a mammalian MPV.

In certain embodiments, the invention provides a method for treating or preventing a respiratory tract infection in a mammal, said method comprising administering a vaccine comprising the recombinant or chimeric mammalian MPV of the invention. In certain embodiments, the invention provides a method for treating or preventing a respiratory tract infection in a mammal, said method comprising administering a vaccine comprising avian MPV. In certain embodiments, the invention provides a method for treating or preventing a respiratory tract infection in a human, said method comprising administering a vaccine comprising avian MPV. In certain embodiments, the invention provides a method for treating or preventing a respiratory tract infection in a subject, said method comprising administering to the subject a composition of the invention.

### 3.1 CONVENTIONS

cDNA complementary DNA
L large protein
M matrix protein (lines inside of envelope)
F fusion glycoprotein
HN hemagglutinin-neuraminidase glycoprotein
N, NP or NC nucleoprotein (associated with RNA and required for polymerase activity)
P phosphoprotein
MOI multiplicity of infection
NA neuraminidase (envelope glycoprotein)
PIV parainfluenza virus
hPIV human parainfluenza virus
hPIV3 human parainfluenza virus type 3
APV/hMPV recombinant APV with hMPV sequences
hMPV/APV recombinant hMPV with APV sequences
Mammalian MPV mammalian metapneumovirus
nt nucleotide
RNP ribonucleoprotein
rRNP recombinant RNP
vRNA genomic virus RNA
cRNA antigenomic virus RNA
hMPV human metapneumovirus
APV avian pneumovirus
MVA modified vaccinia virus Ankara
FACS Fluorescence Activated Cell Sorter
CPE cytopathic effects
Position 1 Position of the first gene of the viral genome to be transcribed
Position 2 Position between the first and the second open reading frame of the native viral genome, or alternatively, the position of the second gene of the viral genome to be transcribed
Position 3 Position between the second and the third open reading frame of the native viral genome, or alternatively, the position of the third gene of the viral genome to be transcribed.
Position 4 Position between the third and the fourth open reading frame of the native viral genome, or alternatively, the position of the fourth gene of the viral genome to be transcribed.
Position 5 Position between the fourth and the fifth open reading frame of the native viral genome, or alternatively, the position of the fifth gene of the viral genome to be transcribed.
Position 6 Position between the fifth and the sixth open reading frame of the native viral genome, or alternatively, the position of the sixth gene of the viral genome to be transcribed.
dpi days post-infection
F protein Fusion protein
HAI hemagglutination-inhibition
HN hemagglutinin-neuraminidase
hpi hours post-infection
MOI multiplicity of infection
POI point of infection
bPIV-3 bovine parainfluenza virus type 3
hPIV-3 human parainfluenza virus type 3
RSV respiratory syncytial virus
SFM serum-free medium
TCID₅₀ 50% tissue culture infective dose
NT nasal turbinates
URT Upper respiratory tract
PRVN plaque reduction virus neutralization

### 4. DESCRIPTION OF THE FIGURES

FIG. 1: Replication kinetics in Vero cell cultures of wild-type hMPV and recombinant viruses in which cp-mutations were introduced. Vero cells, infected at a MOI of 0.1 with hMPV NL/1/99 (panel A), hMPVM₈ (panel B), hMPV_{M11} (panel C), hMPV_{M2} (panel D) or hMPV_{RSV3} (panel E) were washed and incubated for 6 to 8 days at 32° C. (open diamond), 37° C. (closed diamond), 38° C. (open square), 39° C. (closed square) or 40° C. (open triangle). Samples were collected every two days, and virus titers determined by plaque assay.

FIG. 2: Infectious virus titers in (A) nasal turbinates and (B) lungs of Syrian golden hamsters inoculated with 10⁶ TCID₅₀ of NL/1/99, hMPV_{M11} or hMPV_{RSV3}. Nasal turbinates and lungs were collected at 4 dpi. Virus in tissues was quantified by serial dilution in Vero-118 monolayers. The lower limit of detection is indicated with the dotted line.

FIG. 3: 50% Plaque reduction virus neutralization (PRVN) titers measured against NL/1/99, after immunization with NL/1/99, hMPV_{M11} or hMPV_{RSV3}. Blood samples were collected by orbital puncture at 21 dpi. Titers were calculated according to the method of Reed and Muench. The lower limit of detection is indicated with the dotted line.

FIG. 4: Infectious virus titers in (A) nasal turbinates and (B) lungs of Syrian golden hamsters. Animals were immunized with PBS, NL/1/99, hMPV_{M11} or hMPV_{RSV3}. Three weeks after immunization, animals were challenged with 10⁷ TCID₅₀ of the heterologous virus hMPV NL/1/00. Animals were euthanized at 4 dpi. Virus present in tissues was quantified by serial dilution in Vero-118 monolayers. The lower limit of detection is indicated with the dotted line.

FIG. 5: Replication of vRNA-like molecules by polymerase complexes of homologous or heterologous viruses. VRNA-like molecules were co-transfected into BSR-T7 cells with N, P, L and M2.1 expression plasmids and a plasmid expressing β-galactosidase. The means and standard deviations of three independent transfection experiments are given. CAT values are standardized to 10 ng β-galactosidase.

FIG. 6: Replication of vRNA-like molecules by chimeric metapneumovirus polymerase complexes. VRNA-like molecules were co-transfected into BSR-T7 cells with their own N, P, L and M2.1 expression plasmids (black bars) chimeric sets of expression plasmids (grey bars), or the heterologous set of expression plasmids (white bars) and a plasmid expressing β-galactosidase. Plasmids supplied from a heterologous virus species are indicated along the x-axis. The means and standard deviations of three independent transfection experiments are given. CAT values are standardized to 10 ng β-galactosidase.

FIG. 7: Replication kinetics of chimeric hMPV-B1/hMPV-A1 viruses. Vero-118 cells, infected at a multiplicity of infection of 0.1 with hMPV-B1(), hMPV-B1/N_{hMPV-A1} (▾) hMPV-B1/P_{hMPV-A1} (∇), hMPV-B1/NP_{hMPV-A}1 (▪), hMPV-B1/M2.1_{hMPV-A1} (□), hMPV-B1/L_{hMPV-A1}(♦) and hMPV-A1(∘) were washed and incubated. Supernatants were collected daily and virus titers were determined by plaque assay.

FIG. 8: Replication kinetics of chimeric hMPV-B1/aMPV-C viruses. Vero-118 cells, infected at a multiplicity of infection of 0.1 with hMPV-B1 (∘), hMPV-B1/N_{aMPV-C}(▪), hMPV-B1/P_{aMPV-C} (∇), hMPV-B1/L_{aMPV-C} (v), and aMPV-C () were washed and incubated. Supernatants were collected daily and virus titers were determined by plaque assay.

FIG. 9: Evaluation of attenuation of hMPV-B1/aMPV-C chimeric viruses in Syrian golden hamsters. Infectious virus titers were determined in (A) NT and (B) lungs of hamsters inoculated with hMPV-B1, hMPV-B1/N_{aMPV-C}, hMPV-B1/P_{aMPV-C}, hMPV-B1/L_{aMpV-C}, and aMPV-C. NT and lungs were collected four days after inoculation. The lower limit of detection is indicated with the dotted line.

FIG. 10: Aligmnent of hMPV-A1, hMPV-B1, aMPV-C, aMPV-A, RSV, and PIV-3 leader and trailer sequences. Differences in sequence identity are underscored.

FIG. 11: Titers of hMPV in the lungs (A) and nasal turbinates (B) of hamsters immunized with the F protein of hMPV NL/1/99 or NL/1/00. Animals in groups of 8 were immunized with 10 μg of the F protein of NL/1/99 (F1/99) or NL/1/00 (F1/00) along with Specol, with 1M (iscom matrix), or without adjuvant. Control groups consisting of 6 animals each were immunized with Specol alone, IM alone, or PBS. Immunizations were administered twice, with a 3 week interval between them. Three weeks after the second immunization, all animals were challenged with 10⁶ TCID50 of hMPV strain NL/1/00. Four days following challenge, animals were sacrificed, and lungs and nasal turbinates were collected and subjected to virus titration on Vero cells.

FIG. 12: Growth curve of hMPV isolate NL/1/00 (A1) in Vero cells. The Vero cells were infected at a MOI of 0.1.

FIG. 13: Sequence of CAT-hMPV minireplicon construct. The function encoded by a segment of sequence is indicated underneath the sequence.

FIG. 14: Leader and Trailer Sequence Comparison: Alignments of the leader and trailer sequences of different viruses as indicated are shown.

FIG. 15: Expression of CAT from the CAT-hMPV minireplicon. The different constructs used for transfection are indicated on the x-axis; the amount of CAT expression is indicated on the y-axis. The Figure shows CAT expression 24 hours after transfection and CAT expression 48 hours after transfection. Standards were dilutions of CAT protein.

FIG. 16: hMPV genome analysis: PCR fragments of hMPV genomic sequence relative to the hMPV genomic organization are shown. The position of mutations are shown underneath the vertical bars indicating the PCR fragments.

FIG. 17: Restriction maps of hMPV isolate NL/1/00 (A1) and hMPV isolate NL/1/99 (B1). Restriction sites in the respective isolates are indicated underneath the diagram showing the genomic organization of hMPV. The scale on top of the diagram indicates the position in the hMPV genome in kb.

FIGS. 18A and 18B: hMPV cDNA assembly. The diagram on top shows the genomic organization of hMPV, the bars underneath indicate the PCR fragments (see FIG. 27) that are assembled to result in a full length cDNA encoding the virus. The numbers on top of the bars representing the PCR fragments indicate the position in the viral genome in basepairs.

FIG. 19: Results of RT-PCR assays on throat and nose swabs of 12 guinea pigs 15 inoculated with NL/1/00 (A 1) and/or NL/1/99 (B1).

FIG. 20: Comparison of the use of the hMPV ELISA and the APV inhibition ELISA for the detection of IgG antibodies in human sera.

FIG. 21: Generation of M2 deletion mutants. To construct M2 deletions, BspEI sites were constructed at nucleotides 4741 and 5444 and the intervening nucleotides were deleted. To construct M2-1 deletions, NheI sites were constructed at nucleotides 4744 and 5241 and the intervening nucleotides were deleted. To construct M2-2 deletions, SwaI sites were constructed at nucleotides 5311 and 5435 and the intervening nucleotides were deleted.

FIG. 22. Growth curves of recombinant hMPV/NL/1/00 in the presence and absence of Trypsin. wt hMPV=wild type hMPV/NL/1/00; rec hMPV (#21)=recombinant virus with the sequence of hMPV/NL/1/00; rec hMPV (C4A)(#5) recombinant virus with the sequence of hMPV/NL/1/00.

FIG. 23. Replication of wild type and recombinant hMPV in the upper and lower respiratory tract of hamsters.

FIG. 24. Growth curves of wild-type hMPV/NL/1/00 and recombinant hMPV (C4A).

### 5. DETAILED DESCRIPTION OF THE INVENTION

### 5.1 Mutant Mammalian Metapneumovirus

The invention relates to mutants of mammalian metapneumovirus (mMPV). In certain aspects of the invention, the mammalian metapneumovirus is a human metapneumovirus (hMPV). The mammalian MPV can be a variant A1, A2, B1 or B2 mammalian MPV. In certain embodiments, the mutant mMPV or hMPV is attenuated and can be used as a vaccine. In certain embodiments, the mutant mMPV or hMPV of the invention can be used in an immunogenic composition. In certain embodiments, the mutant mMPV or hMPV is temperature-sensitive. In certain embodiments, the mutant viruses of the invention are generation using recombinant DNA technology.

In certain embodiments, a mutant mMPV of the invention alters the host specificity, replication efficiency, efficiency of infectivity, efficiency of viral mRNA transcription, efficiency of viral protein synthesis, efficiency of assembly and release of the mutant mMPV relative to wild type mMPV.

In accordance with the present invention, a recombinant virus is one derived from a mammalian MPV or an APV that is encoded by endogenous or native genomic sequences or non-native genomic sequences. In accordance with the invention, a non-native sequence is one that is different from the native or endogenous genomic sequence due to one or more mutations, including, but not limited to, point mutations, rearrangements, insertions, deletions etc., the genomic sequence that may or may not result in a phenotypic change. In accordance with the invention, a chimeric virus is a recombinant MPV or APV which further comprises a heterologous nucleotide sequence. In accordance with the invention, a chimeric virus may be encoded by a nucleotide sequence in which heterologous nucleotide sequences have been added to the genome or in which endogenous or native nucleotide sequences have been replaced with heterologous nucleotide sequences.

In certain embodiments, the replication rate of the recombinant virus of the invention is at most 5%, at most 10%, at most 20%, at most 30%, at most 40%, at most 50%, at most 75%, at most 80%, at most 90% of the replication rate of the wild type virus from which the recombinant virus is derived under the same conditions. In certain embodiments, the replication rate of the recombinant virus of the invention is at least 5%, at least 10%, at least 20%, at least 30%, at least 40%, at least 50%, at least 75%, at least 80%, at least 90% of the replication rate of the wild type virus from which the recombinant virus is derived under the same conditions. In certain embodiments, the replication rate of the recombinant virus of the invention is between 5% and 20%, between 10% and 40%, between 25% and 50%, between 40% and 75%, between 50% and 80%, or between 75% and 90% of the replication rate of the wild type virus from which the recombinant virus is derived under the same conditions.

The mutant viruses of the invention can be used in pharmaceutical compositions, in immunogenic compositions, and in vaccines. The mutant viruses of the invention can be used as expression vectors of non-native nucleotide sequences (i.e., non-native to the mMPV). See section 5.5. Such expression vectors can be used to express protein in different expression systems or as immunogenic compositions to stimulate the immune system against the non-native protein.

In certain embodiments, an isolated mammalian MPV is provided which comprises a genetic modification resulting in an amino acid substitution, deletion, or insertion at one or more amino acid positions selected from the group consisting of: position 66 in the protein; positions 9, 38, 52, and 132 in the M protein; positions 93, 101, 280, 471, 532, and 538 in the F protein; position 187 in the M2 protein; positions 139 and 164 in the G protein; and positions 235, 323, and 1453 in the L protein, with the proviso that the modification at position 101 in the F protein is not a substitution to Proline and that the modification at position 93 in the F protein is not a substitution to Lysine. In a more specific embodiment, the genetic modification results in an amino acid substitution, deletion, or insertion at one or more amino acid positions selected from the group consisting of: position 66 in the P protein; position 132 in the M protein; positions 101, 280, and 471 in the F protein; position 187 in the M2 protein; position 139 in the G protein; and positions 235, 323, and 1453 in the L protein, with the proviso that modification at position 101 in the F protein is not a substitution to Proline. In another specific embodiment, the genetic modification results in an amino acid substitution, deletion, or insertion at one or more amino acid positions selected from the group consisting of: position 132 in the M protein; positions 101, 280, and 471 in the F protein; position 187 in the M2 protein; position 139 in the G protein; and position 1453 in the L protein, with the proviso that modification at position 101 in the F protein is not a substitution to Proline. In a further embodiment, the genetic modification results in an amino acid substitution, deletion, or insertion at one or more amino acid positions selected from the group consisting of: positions 235 and 323 in the L protein. In a specific embodiment, the isolated mammalian metapneumovirus comprises genetic modifications resulting in amino acid substitution, deletion, or insertion at amino acid positions 235 and 323 in the L protein.

In certain embodiments, the isolated mammalian MPV of the invention further comprises a genetic modification that is a silent mutation, i.e., it does not result in an amino acid exchange. In more specific embodiments, the isolated mammalian MPV comprises a silent mutation at one or more of the nucleotide positions selected from the group consisting of: positions 336 and 436 in the M open reading frame.

In certain embodiments, the isolated mammalian MPV of the invention further comprises a genetic alteration that results in an amino acid exchange at amino acid 109 of the F protein. In a more specific embodiment, the isolated mammalian MPV of the invention further comprises a mutation at nucleotide position 325 of the F protein that results in a serine at that position. In a more specific embodiment, the isolated mammalian MPV of the invention further comprises a mutation at nucleotide position 325 of the F protein.

In another embodiment, the invention provides an isolated mammalian MPV comprising a genetic modification at one or more of the nucleotide positions selected from the group consisting of: position 197 in the P open reading frame; position 9, 113, 155, 336, 394, and 436 in the M open reading frame; positions 277, 301, 839, 1412, 1594, and 1612 in the F open reading frame; position 560 in the M2 open reading frame; position 415 and 491 in the G open reading frame; and positions 703, 967, and 4357 in the L open reading frame.

In a further embodiment, the invention provides an isolated mammalian MPV comprising a genetic modification resulting in one or more amino acid changes selected from the group consisting of: (i) position 66 in the P gene is altered to Val; (ii) position 9 in the M gene is altered to His; (iii) position 38 in the M gene is altered to Ser; (iv) position 52 in the M gene is altered to Pro; (v) position 132 in the M gene is altered to Pro; (vi) position 93 in the F gene is altered to Lys; (vii) position 280 in the F gene is altered to Gly; (viii) position 471 in the F gene is altered to Arg; (ix) position 532 in the F gene is altered to Tyr; (x) position 538 in the F gene is altered to Tyr; (xi) position 187 in the M2 gene is altered to Ile; (xii) position 139 in the G gene is altered to Pro; (xiii) position 164 in the G gene is altered to Pro; (xiv) position 235 in the L gene is altered to Arg; (xv) position 323 in the L gene is altered to Asp; and (xvi) position 1453 in the L gene is altered to Leu.

In another embodiment, the invention provides an isolated mammalian MPV comprising a genetic modification resulting in one or more amino acid changes selected from the group consisting of: (i) position 66 in the P gene is altered to Val; (ii) position 9 in the M gene is altered to His; (iii) position 38 in the M gene is altered to Ser; (iv) position 52 in the M gene is altered to Pro; (v) position 132 in the M gene is altered to Pro; (vi) position 93 in the F gene is altered to Lys; (vii) position 280 in the F gene is altered to Gly; (viii) position 471 in the F gene is altered to Arg; (ix) position 532 in the F gene is altered to Tyr; (x) position 538 in the F gene is altered to Tyr; (xi) position 187 in the M2 gene is altered to Ile; (xii) position 139 in the G gene is altered to Pro; (xiii) position 164 in the G gene is altered to Pro; (xiv) position 235 in the L gene is altered to Arg; (xv) position 323 in the L gene is altered to Asp; and (xvi) position 1453 in the L gene is altered to Leu, wherein the isolated mammalian MPV comprising genetic modifications at any of (i) to (xvi) also has genetic modifications at one or more of the nucleotide positions selected from the group consisting of: positions 336 and 436 in the M open reading frame, wherein the genetic modifications at positions 336 and 436 in the M open reading frame result in silent mutations.

In embodiments of the invention wherein isolated mammalian MPV comprising several potential amino acid modifications are provided, the isolated mammalian MPV may have at least two, at least three, at least four, at least five, at least six, at least seven or at least eight of the specified genetic modifications.

In another embodiment, the invention provides for a recombinant mammalian MPV comprising two or more genetic modifications, wherein the genetic modification is an amino acid substitution, deletion, or insertion amino acid position 456 of the L gene; position 1094 of the L gene; or position 1246 of the L gene; or a nucleotide substitution, deletion, or insertion at the gene start sequence of the M2 gene.

In yet another embodiment, the invention provides for a recombinant mammalian MPV, comprising an alteration in the gene start sequence of the M2 gene; an alteration in the L gene such that Phe at amino acid position 456 is mutated to Leu; and an alteration of the L gene such that Met at amino acid position 1094 is mutated to Val.

In still other embodiments, an isolated mammalian MPV is provided which comprises a genetic modification resulting in an amino acid substitution, deletion, or insertion at one or more amino acid positions selected from the group consisting of: amino acid position 129 of the M gene, amino acid positions 129, 231, 294, 307, 475 and 488 of the F protein, amino acid position 35 of the SH protein, amino acid positions 113 and 133 of the G protein, and amino acid positions 403, 537, 1220, 1336, 1440 and 1997 of the L protein. In a further aspect of this embodiment, the isolated mammalian MPV comprises a genetic modification at one or more genomic nucleotide positions selected from the group consisting of: genomic nucleotide positions 6072 and 6076 in the gene end sequence of the SH protein. In another aspect of this embodiment, the isolated mammalian MPV of the invention further comprises a genetic modification that is a silent mutation, i.e., it does not result in an amino acid exchange. In a more specific embodiment, the isolated mammalian MPV comprises a silent mutation at one of the nucleotide positions in the codon of one or more of the amino acids positions selected from the group consisting of: amino acid position 177 in the G protein and amino acid positions 554, 568, 582, 819, and 1343 in the L protein. In another embodiment, the isolated mammalian MPV of the invention comprises all of these mutations. In a specific aspect of this embodiment, the isolated mammalian MPV is strain NL/1/94.

In another embodiment, an isolated mammalian MPV is provided which comprises a genetic modification resulting in an amino acid substitution, deletion, or insertion at one or more amino acid positions selected from the group consisting of: amino acid positions 341 and 465 of the F protein, amino acid position 119 of the M2.1 protein, and amino acid position 467 of the L protein. In a further aspect of this embodiment, the isolated mammalian MPV comprises a genetic modification at one or more genomic nucleotide positions selected from the group consisting of: genomic nucleotide position 27 in the leader sequence, genomic nucleotide position 4692 in the gene end sequence of the F protein, and genomic nucleotide position 6981 in the gene end sequence of the G protein. In a more specific embodiment, the isolated mammalian MPV comprises a silent mutation at one of the nucleotide positions in the codon of one or more of the amino acids positions selected from the group consisting of: amino acid positions 1206, 1402, and 1407 in the L protein. In another embodiment, the isolated mammalian MPV of the invention comprises all of these mutations. In a specific aspect of this embodiment, the isolated mammalian MPV is strain NL/17/00.

In yet another embodiment, an isolated mammalian MPV is provided which comprises a genetic modification resulting in an amino acid substitution, deletion, or insertion at one or more amino acid positions selected from the group consisting of: amino acid position 130 of the M protein, amino acid positions 93, 100, and 101 of the F protein, amino acid position 10 of the G protein, and amino acid position 1138 of the L protein. In another embodiment, an isolated mammalian MPV is provided which comprises a genetic modification resulting in an amino acid substitution, deletion, or insertion at one or more amino acid positions selected from the group consisting of: amino acid position 130 of the M protein, amino acid positions 100, 101, 468, and 529 of the F protein, amino acid position 45 of the M2.2 protein, and amino acid position 10 of the G protein. In a further aspect of this embodiment, the isolated mammalian MPV comprises a genetic modification at one or more genomic nucleotide positions selected from the group consisting of: genomic nucleotide position 13306 in the trailer sequence. In a more specific embodiment, the isolated mammalian MPV comprises a silent mutation at one of the nucleotide positions in the codon of one or more of the amino acids positions selected from the group consisting of: amino acid position 93 of the F protein, amino acid position 90 of the SH protein, and amino acid positions 270, 736, 689, and 1138 of the L protein. In another embodiment, the isolated mammalian MPV of the invention comprises all of these mutations. In a specific aspect of this embodiment, the isolated mammalian MPV is strain NL/1/00.

In certain embodiments of the invention the mutant isolated mammalian MPV carries an amino acid exchange that is encoded by two or three nucleotide substitutions per codon, i.e., a stabilized codon.

In embodiments of the invention comprising isolated mammalian MPV, the isolated mammalian MPV may be temperature-sensitive. In certain embodiments, the isolated mammalian MPV may be a human MPV. In more specific embodiments, the isolated mammalian MPV may be hMPV variant A1, A2, B1, or B2. In other specific embodiments, the isolated mammalian MPV may be hMPV strain NL/1/99, NL/17/00, NL/1/00, or NL/1/94.

In another embodiment of the invention, a method is provided for stimulating the immune response against mammalian MPV in a mammal comprising administering to the mammal an isolated mammalian MPV of the invention including, but not limited to, the isolated mammalian MPV comprising the genetic modifications described above. In one aspect of this embodiment, the mammal is a human. In another aspect of this embodiment, the isolated mammalian MPV is a human MPV, wherein the hMPV can in some aspects be hMPV variant A1, A2, B1, or B2. In other aspects of this embodiment, the hMPV can be hMPV strain NL/1/99, NL/1/00, NL/17/00, or NL/1/94.

### 5.2 Chimeric Viral Polymerases and Assays

The invention also provides an assay to determine the activity of an RNA-directed chimeric RNA polymerase complex. This assay is also suited for determining the activity of an RNA polymerase complex that is from a virus other than the virus being replicated. In certain embodiments, the RNA polymerase complex is from a virus different from the virus whose genomic termini are replicated. This assay can be used to determine the specificity of an RNA polymerase complex for a particular virus as substrate.

In certain embodiments, the invention provides an assay for determining the activity of an RNA-directed RNA polymerase complex wherein the substrate of the RNA polymerase complex is a minigenome (i.e., a reporter gene flanked by the genomic termini of a virus) with genomic termini of a virus different from the virus from which the RNA polymerase was obtained.

This assay can be used to determine which combinations of RNA polymerase subunits are suitable to replicate a virus at lower levels to result in a replication-competent, yet attenuated, virus. The subunits of the RNA polymerase complex or the chimeric RNA polymerase complex can be mutated. In a specific embodiment, one or more subunits of the chimeric RNA polymerase complex are from mMPV. In an even more specific embodiment, one or more of the mMPV subunits carries one or more of the mutations of a virus of the invention (see Section 5.1).

The assay can be performed as discussed for the minireplicon constructs in section 5.8 (a).

In certain embodiments, the subunits of the chimeric RNA polymerase complex are the N, P, L, and M2.1 proteins. The individual components are from two, three, or four different viruses of the family of paramyxoviridae. In more specific embodiments, at least one subunit is from mMPV, RSV, PIV, measles virus, mumps virus, or avian metapneumovirus. In other embodiments, at least one RNA polymerase complex subunit is from a Mononegavirales other than a paramyxoviridae. In certain embodiments, the different subunits are derived from different variants of mMPV, i.e., A1, A2, B1, and/or B2.

In certain embodiments, the genomic termini of the substrate of the RNA polymerase complex are from a member of the paramyxoviridae, such as, but not limited to, mMPV, RSV, PIV, measles virus, mumps virus, or avian metapneumovirus.

In certain embodiments, a host cell is transfected with nucleic acids encoding the individual components of the viral RNA polymerase complex and with a nucleic acid encoding the minireplicon. The subunits and the replicon can be transcribed by a DNA-directed RNA polymerase, such as, but not limited to T3, T7, or Sp6. The host cell can be transiently transfected or stably transfected with DNA encoding the DNA-directed RNA polymerase, such as, but not limited to T3, T7, or Sp6. For example, Vero cells can be engineered to express T7 RNA polymerase under the control of a CMV or SV40 promoter. This approach is useful because it eliminates the need for co-infection with a helper virus, such as a pox-virus expressing T7 RNA polymerase. Another advantage of this method is the elimination of the need for selection systems required to remove the helper virus.

Alternatively, the host cell is infected with Modified Vaccinia Virus Ankara (MVA) encoding T7 RNA polymerase. The following cells can be used as hosts: Vero cells, LLC-MK-2 cells, HEp-2 cells, LF 1043 (HEL) cells, tMK cells, LLC-MK2, HUT 292, FRHL-2 (rhesus), FCL-1 (green monkey), WI-38 (human), MRC-5 (human) cells, 293 T cells, QT 6 cells, QT 35 cells and CEF cells. The reporter gene can be a viral gene, CAT (chloramphenicol acetyltransferase—transfers radioactive acetyl groups to chloramphenicol or detection by thin layer chromatography and autoradiography); GAL (b-galactosidase—hydrolyzes colorless galactosides to yield colored products); GUS (b-glucuronidase—hydrolyzes colorless glucuronides to yield colored products); LUC (luciferase—oxidizes luciferin, emitting photons); GFP (green fluorescent protein—fluorescent protein without substrate); SEAP (secreted alkaline phosphatase—luminescence reaction with suitable substrates or with substrates that generate chromophores); HRP (horseradish peroxidase—in the presence of hydrogen oxide, oxidation of 3,3′,5,5′-tetramethylbenzidine to form a colored complex); and AP (alkaline phosphatase—luminescence reaction with suitable substrates or with substrates that generate chromophores). See section 5.8(b).

The amount of reporter gene expressed or the activity of the expressed reporter gene can be determined by any method known to the skilled artisan. For the amount, transcribed RNA can be detected and quantified by Northern blotting, PCR analysis, real time PCR analysis, molecular beacons etc. Expressed protein can be detected and quantified by, e.g., Western blotting and immunoprecipitation. Peptide tags can also be used to quantify the expressed reporter gene. The activity of the expressed reporter gene can be detected and quantified based on the enzymatic properties of the reporter gene. See section 5.8(b).

The amount/activity of the expressed reporter gene is a measure for the activity of the RNA-directed RNA polymerase complex or the chimeric RNA-directed RNA polymerase complex. The higher the amount/activity of the expressed reporter gene, the higher the activity of the RNA-directed RNA polymerase complex or the chimeric RNA-directed RNA polymerase complex. Vice versa, the lower the amount/activity of the expressed reporter gene, the lower the activity of the RNA-directed RNA polymerase complex or the chimeric RNA-directed RNA polymerase complex.

The specificity (attributes to heterologous viruses) and the effect of the terminal residues of the leader (attributes to homologous virus) of the minireplicon system can also be tested by superinfecting the minireplicon-transfected cells with hMPV polymerase components (NL/1/00 and NL/1/99) or polymerase components from APV-A, APV-C, RSV or PIV. The different amount of each of the six plasmids can also be tested in order to determine the optimal conditions.

### 5.3 Mammalian Metapneumovirus

Any mammalian metapneumovirus (mMPV) can be used for the generation of the mutant viruses of the invention, for the chimeric viruses and RNA polymerase complexes of the invention, and for the methods of the invention. In specific embodiments, human metapneumovirus (hMPV) can be used for the generation of the mutant viruses of the invention, for the chimeric viruses and RNA polymerase complexes of the invention, and for the methods of the invention.

mMPV is an isolated negative-sense single stranded RNA virus MPV belonging to the sub-family Pneumovirinae of the family Paramyxoviridae and identifiable as phylogenetically corresponding to the genus Metapneumovirus, wherein the virus is phylogenetically more closely related to a virus isolate deposited as 1-2614 with CNCM, Paris than to turkey rhinotracheitis virus, the etiological agent of avian rhinotracheitis.

mMPV can be devided into two subgroups: subgroup A and subgroup B. The mammalian MPVs can be a variant A1, A2, B1 or B2 mammalian MPV. A mammalian MPV can be identified as a member of subgroup A if it is phylogenetically closer related to the isolate NL/1/00 (SEQ ID NO:2) than to the isolate NL/1/99 (SEQ ID NO:1). A mammalian MPV can be identified as a member of subgroup B if it is phylogenetically closer related to the isolate NL/1/99 (SEQ ID NO: 1) than to the isolate NL/1/00 (SEQ ID NO:2).

The isolate NL/1/00 (SEQ ID NO:2) is an example of the variant A1 of mammalian MPV. The isolate NL/1/99 (SEQ ID NO: 1) is an example of the variant B1 of mammalian MPV. An isolate of mammalian MPV is classified as a variant B1 if it is phylogenetically closer related to the viral isolate NL/1/99 (SEQ ID NO: 1) than it is related to any of the following other viral isolates: NL/1/00 (SEQ ID NO:2), NL/17/00 (SEQ ID NO:3) and NL/1/94 (SEQ ID NO:4). An isolate of mammalian MPV is classified as a variant A1 if it is phylogenetically closer related to the viral isolate NL/1/00 (SEQ ID NO:2) than it is related to any of the following other viral isolates: NL/1/99 (SEQ ID NO: 1), NL/17/00 (SEQ ID NO:3) and NL/1/94 (SEQ ID NO:4). An isolate of mammalian MPV is classified as a variant A2 if it is phylogenetically closer related to the viral isolate NL/17/00 (SEQ ID NO:3) than it is related to any of the following other viral isolates: NL/1/99 (SEQ ID NO: 1), NL/1/00 (SEQ ID NO:2) and NL/1/94 (SEQ ID NO:4). An isolate of mammalian MPV is classified as a variant B2 if it is phylogenetically closer related to the viral isolate NL/1/94 (SEQ ID NO:4) than it is related to any of the following other viral isolates: NL/1/99 (SEQ ID NO:1), NL/1/00 (SEQ ID NO:2) and NL/17/00 (SEQ ID NO:3).

The classification of an mMPV into one of the variants, A1, A2, B1, and B2, can be based on nucleotide sequence of amino acid sequence identity of one or more genes, non-coding regions, and/or proteins. For example, the N, P, M, F, M2, SH, G, or L protein of an A1 mMPV is at least 90%, 95%, 98%, 99% or 99.5% identical to the corresponding protein of NL/1/00 (SEQ ID NO:2). For example, the N, P, M, F, M2, SH, G, or L protein of an A2 mMPV is at least 90%, 95%, 98%, 99% or 99.5% identical to the corresponding protein of NL/17/00 (SEQ ID NO:3). For example, the N, P, M, F, M2, SH, G, or L protein of a B1 mMPV is at least 90%, 95%, 98%, 99% or 99.5% identical to the corresponding protein of NL/1/94 (SEQ ID NO:4). For example, the N, P, M, F, M2, SH, G, or L protein of a B2 mMPV is at least 90%, 95%, 98%, 99% or 99.5% identical to the corresponding protein of NL/1/99 (SEQ ID NO: 1).

See Table 1 for a description of the sequences for each sequence identifier number.

mMPV, such as hMPV, and methods for identifying mMPV, such as hMPV, are disclosed in International Patent Application PCT/NL02/00040, published as WO 02/057302, which is incorporated by reference in its entirety herein. In particular, PCT/NL02/00040 discloses nucleic acid sequences that are suitable for phylogenetic analysis at page 12, line 27 to page 19, line 29, which are incorporated by reference herein.

Additional descriptions of mMPV, such as hMPV, can be found in International Patent Application No PCT/US03/05271 (published as WO 03/072719) and International Patent Application No. PCT/US04/12724 (published as WO 04/096993), both of which are incorporated herein by reference in their entireties. In particular, these international patent application publications describe the variants A1, A2, B1, and B2 of mMPV.

### 5.4 Recombinant and Chimeric Metapneumovirus

In certain embodiments, a mutant MPV of the invention further comprises a non-native nucleotide sequence. In accordance with the invention, a chimeric virus may be encoded by a nucleotide sequence in which the non-native nucleotide sequence has been added to the genome or in which an endogenous or native nucleotide sequence has been replaced with heterologous nucleotide sequence.

The non-native nucleotide sequence can be from a different strains of mMPV. The non-native nucleotide sequence can encode a polypeptide, or it may be a non-coding sequence. Non-native nucleotide sequences to be incorporated into the viral genome include sequences obtained or derived from different strains of metapneumovirus, a different variant of MPV, i.e., variant A1, A2, B1, or B2, strains of avian pneumovirus, and other negative strand RNA viruses, including, but not limited to, RSV, PIV and influenza virus, HIV (e.g., the gp 160 protein), and other viruses, including morbillivirus. A non-native sequence may encode a tag or marker or a biological response modifier, examples of which include, lymphokines, interleukines, granulocyte macrophage colony stimulating factor and granulocyte colony stimulating factor, cytokines, interferon type 1, gamma interferon, colony stimulating factors, and interleukin −1, −2, −4, −5, −6, −12, or a chimeric F or G protein of RSV, PIV, APV or hMPV. For heterologous nucleotide sequences derived from respiratory syncytial virus see, e.g., PCT/US98/20230, which is hereby incorporated by reference in its entirety.

Thus, the mutant virus of the invention that carries a non-native sequence may express a protein from a different virus or organism. Such chimeric mutant mMPV can be used as immunogenic compositions or as vaccines to stimulate an immune response against the mMPV and against the other virus or organism. The expression products and/or recombinant or chimeric virions obtained in accordance with the invention may advantageously be utilized in vaccine formulations. The expression products and chimeric virions of the present invention may be engineered to create vaccines against a broad range of pathogens, including viral and bacterial antigens, tumor antigens, allergen antigens, and auto antigens involved in autoimmune disorders. In particular, the chimeric virions of the present invention may be engineered to create vaccines for the protection of a subject from infections with PIV, RSV, and/or metapneumovirus.

Non-native gene sequences that can be expressed into the recombinant viruses of the invention include but are not limited to antigenic epitopes and glycoproteins of viruses which result in respiratory disease, such as influenza glycoproteins, in particular hemagglutinin H5, H7, respiratory syncytial virus epitopes, New Castle Disease virus epitopes, Sendai virus and infectious Laryngotracheitis virus (ILV). Non-native nucleotide sequences can be from a RSV or PIV. Non-native gene sequences that can be engineered into the chimeric viruses of the invention include, but are not limited to, viral epitopes and glycoproteins of viruses, such as hepatitis B virus surface antigen, hepatitis A or C virus surface glycoproteins of Epstein Barr virus, glycoproteins of human papilloma virus, simian virus 5 or mumps virus, West Nile virus, Dengue virus, glycoproteins of herpes viruses, VPI of poliovirus, and sequences derived from a lentivirus, preferably, but not limited to human immunodeficiency virus (HIV) type 1 or type 2. In yet another embodiment, heterologous gene sequences that can be engineered into chimeric viruses of the invention include, but are not limited to, Marek's Disease virus (MDV) epitopes, epitopes of infectious Bursal Disease virus (IBDV), epitopes of Chicken Anemia virus, infectious laryngotracheitis virus (ILV), Avian Influenza virus (AIV), rabies, feline leukemia virus, canine distemper virus, vesicular stomatitis virus, and swinepox virus (see Fields et al., (ed.), 1991, Fundamental Virology, Second Edition, Raven Press, New York, incorporated by reference herein in its entirety).

In certain embodiments, the non-native nucleotide sequence encodes an F protein or a G protein or a fragment of an F protein or a G protein. In an exemplary embodiment, the F-gene and/or the G-gene of human metapneumovirus have been replaced with the F-gene and/or the G-gene of avian pneumovirus to construct chimeric hMPV/APV virus. In other embodiments, viral vectors contain sequences derived from APV and mammalian MPV, such that a chimeric APV/hMPV virus is encoded by the viral vector. In more exemplary embodiments, the F-gene and/or the G-gene of avian pneumovirus have been replaced with the F-gene and/or the G-gene of human metapneumovirus to construct the chimeric APV/hMPV virus.

In another embodiment, the chimeric virions of the present invention may be engineered to create anti-HIV vaccines, wherein an immunogenic polypeptide from gp 160, and/or from internal proteins of HIV is engineered into the glycoprotein HN protein to construct a vaccine that is able to elicit both vertebrate humoral and cell-mediated immune responses. In yet another embodiment, the invention relates to recombinant metapneumoviral vectors and viruses which are engineered to encode mutant antigens. A mutant antigen has at least one amino acid substitution, deletion or addition relative to the wild-type viral protein from which it is derived. In instances whereby the heterologous sequences are HIV-derived, such sequences may include, but are not limited to sequences derived from the env gene (i.e., sequences encoding all or part of gp160, gp120, and/or gp41), the pol gene (i.e., sequences encoding all or part of reverse transcriptase, endonuclease, protease, and/or integrase), the gag gene (i.e., sequences encoding all or part of p 7, p 6, p 55, p 17/18, p 24/25) tat, rev, nef, vif, vpu, vpr, and/or vpx.

Mutant mMPV of the invention may be engineered to express tumor-associated antigens (TAAs), including but not limited to, human tumor antigens recognized by T cells (Robbins and Kawakami, 1996, Curr. Opin. Immunol. 8:628-636, incorporated herein by reference in its entirety), melanocyte lineage proteins, including gp100, MART-1/MelanA, TRP-1 (gp75), tyrosinase; Tumor-specific widely shared antigens, MAGE-1, MAGE-3, BAGE, GAGE-1, GAGE-1, N-acetylglucosaminyltransferase-V, p 15; Tumor-specific mutated antigens, β-catenin, MUM-1, CDK4; Nonmelanoma antigens for breast, ovarian, cervical and pancreatic carcinoma, HER-2/neu, human papillomavirus-E6, -E7, MUC-1.

The non-native sequence can be from Salmonella, Shigella, Chlamydia, Helicobacter, Yersinia, Bordatella, Pseudomonas, Neisseria, Vibrio, Haemophilus, Mycoplasma, Streptomyces, Treponema, Coxiella, Ehrlichia, Brucella, Streptobacillus, Fusospirocheta, Spirillum, Ureaplasma, Spirochaeta, Mycoplasma, Actinomycetes, Borrelia, Bacteroides, Trichomoras, Branhamella, Pasteurella, Clostridium, Corynebacterium, Listeria, Bacillus, Erysipelothrix, Rhodococcus, Escherichia, Klebsiella, Pseudomanas, Enterobacter, Serratia, Staphylococcus, Streptococcus, Legionella, Mycobacterium, Proteus, Campylobacter, Enterococcus, Acinetobacter, Morganella, Moraxella, Citrobacter, Rickettsia, Rochlimeae, as well as bacterial species such as: P. aeruginosa; E. coli, P. cepacia, S. epidermis, E. faecalis, S. pneumonias, S. aureus, N. meningitidis, S. pyogenes, Pasteurella multocida, Treponema pallidum, and P. mirabilis.

Examples of non-native gene sequences derived from pathogenic fungi, include, but are not limited to, antigens derived from fungi such as Cryptococcus neoformans; Blastomyces dermatitidis; Aiellomyces dermatitidis; Histoplasma capsulatum; Coccidioides immitis; Candida species, including C. albicans, C. tropicalis, C. parapsilosis, C. guilliermondii and C. krusei, Aspergillus species, including A. fumigatus, A. flavus and A. niger, Rhizopus species; Rhizomucor species; Cunninghammella species; Apophysomyces species, including A. saksenaea, A. mucor and A. absidia, Sporothrix schenckii, Paracoccidioides brasiliensis; Pseudallescheria boydii, Torulopsis glabrata; Trichophyton species, Microsporum species and Dermatophyres species, as well as any other yeast or fungus now known or later identified to be pathogenic.

Finally, examples of non-native gene sequences derived from parasites include, but are not limited to, antigens derived from members of the Apicomplexa phylum such as, for example, Babesia, Toxoplasma, Plasmodium, Eimeria, Isospora, Atoxoplasma, Cystoisospora, Hammondia, Besniotia, Sarcocystis, Frenkelia, Haemoproteus, Leucocytozoon, Theileria, Perkinsus and Gregarina spp., Pneumocystis carinii; members of the Microspora phylum such as, for example, Nosema, Enterocytozoon, Encephalitozoon, Septata, Mrazekia, Amblyospora, Ameson, Glugea, Pleistophora and Microsporidium spp.; and members of the Ascetospora phylum such as, for example, Haplosporidium spp., as well as species including Plasmodium falciparum, P. vivax, P. ovale, P. malaria; Toxoplasma gondii; Leishmania mexicana, L. tropica, L. major, L. aethiopica, L. donovani, Trypanosoma cruzi, T brucei, Schistosoma mansoni, S. haematobium, S. japonium, Trichinella spiralis; Wuchereria bancrofti, Brugia malayli; Entamoeba histolytica; Enterobius vermiculoarus; Taenia solium, T. saginata, Trichomonas vaginatis, T. hominis, T. tenax; Giardia lamblia, Cryptosporidium parvum; Pneumocytis carinii, Babesia bovis, B. divergens, B. microti, Isospora belli, L. hominis, Dientamoeba fragilis; Onchocerca volvulus; Ascaris lumbricoides; Necator americanis; Ancylostoma duodenale; Strongyloides stercoralis, Capillaria philippinensis; Angiostrongylus cantonensis, Hymenolepis nana; Diphyllobothrium latum; Echinococcus granulosus, E. multilocularis, Paragonimus westermani, P. caliensis; Chlonorchis sinensis; Opisthorchis felineas, G. Viverini, Fasciola hepatica, Sarcoptes scabiei, Pediculus humanus; Phihirlus pubis; and Dermatobia hominis, as well as any other parasite now known or later identified to be pathogenic.

A chimeric virus may be of particular use for the generation of recombinant vaccines protecting against two or more viruses (Tao et al., J. Virol. 72, 2955-2961; Durbin et al., 2000, J. Virol. 74, 6821-6831; Skiadopoulos et al., 1998, J. Virol. 72, 1762-1768; Teng et al., 2000, J. Virol. 74, 9317-9321). For example, it can be envisaged that a MPV or APV virus vector expressing one or more proteins of another negative strand RNA virus, e.g., RSV or a RSV vector expressing one or more proteins of MPV will protect individuals vaccinated with such vector against both virus infections. A similar approach can be envisaged for PIV or other paramyxoviruses. Attenuated and replication-defective viruses may be of use for vaccination purposes with live vaccines as has been suggested for other viruses. (See, PCT WO 02/057302, at pp. 6 and 23, incorporated by reference herein).

In certain embodiments of the invention, one or more sequences, intergenic regions, termini sequences, or portions or entire ORF have been substituted with a non-native sequence.

In certain embodiments, the non-native nucleotide sequence is inserted or added at Position 1 of the viral genome. In another preferred embodiment, the non-native nucleotide sequence is inserted or added at Position 2 of the viral genome. In even another preferred embodiment, the non-native nucleotide sequence is inserted or added at Position 3 of the viral genome. Insertion or addition of nucleic acid sequences at the lower-numbered positions of the viral genome results in stronger or higher levels of expression of the non-native nucleotide sequence compared to insertion at higher-numbered positions due to a transcriptional gradient across the genome of the virus. Thus, inserting or adding non-native nucleotide sequences at lower-numbered positions is the preferred embodiment of the invention if high levels of expression of the heterologous nucleotide sequence is desired.

The non-native sequence can be inserted at Postion 1, 2, 3, 4, 5, or 6. Without being bound by theory, the position of insertion or addition of the non-native sequence affects the replication rate of the virus. The higher rates of replication can be achieved if the non-native sequence is inserted or added at Position 2 or Position 1 of the viral genome. The rate of replication is reduced if the non-native sequence is inserted or added at Position 3, Position 4, Position 5, or Position 6.

Depending on the purpose (e.g., to have strong immunogenicity) of the inserted non-native nucleotide sequence, the position of the insertion and the length of the intergenic region of the inserted heterologous nucleotide sequence can be determined by various indexes including, but not limited to, replication kinetics and protein or mRNA expression levels, measured by following non-limiting examples of assays: plaque assay, fluorescent-focus assay, infectious center assay, transformation assay, endpoint dilution assay, efficiency of plating, electron microscopy, hemagglutination, measurement of viral enzyme activity, viral neutralization, hemagglutination inhibition, complement fixation, immunostaining, immunoprecipitation and immunoblotting, enzyme-linked immunosorbent assay, nucleic acid detection (e.g., Southern blot analysis, Northern blot analysis, Western blot analysis), growth curve, employment of a reporter gene (e.g., using a reporter gene, such as Green Fluorescence Protein (GFP) or enhanced Green Fluorescence Protein (eGFP), integrated to the viral genome the same fashion as the interested heterologous gene to observe the protein expression), or a combination thereof. Procedures of performing these assays are well known in the art (see, e.g., Flint et al., PRINCIPLES OF VIROLOGY, MOLECULAR BIOLOGY, PATHOGENESIS, AND CONTROL, 2000, ASM Press pp 25-56, the entire text is incorporated herein by reference), and non-limiting examples are given in the Example sections, infra.

In a specific embodiment, the non-native sequence is inserted into the region of the G-ORF that encodes for the ectodomain, such that it is expressed on the surface of the viral envelope. In one approach, the non-native sequence may be inserted within the antigenic site without deleting any viral sequences. In another approach, the non-native sequences replaces sequences of the G-ORF. Expression products of such constructs may be useful in vaccines against the foreign antigen, and may indeed circumvent problems associated with propagation of the recombinant virus in the vaccinated host. An intact G molecule with a substitution only in antigenic sites may allow for G function and thus allow for the construction of a viable virus. Therefore, this virus can be grown without the need for additional helper functions.

Without being bound by theory, the size of the intergenic region between the viral gene and the non-native sequence further determines rate of replication of the virus and expression levels of the heterologous sequence.

In certain embodiments, the viral vector of the invention contains two or more different non-native nucleotide sequences.

### 5.5 Construction of the Recombinant cDNA and RNA

Standard recombinant DNA technology can be used to generate a cDNA encoding a mutant virus of the invention. The cDNA can optionally contain one or more non-native nucleotide sequences. See Section 5.4.

In certain embodiments, the starting material is a cDNA of the sequence of SEQ ID NO: 1, 2, 3, or 4. Mutations can be introduced into the cDNA by any method known to the skilled artisan. Such methods include PCR amplification using primers encoding the mutation. Exemplary mutagenic primers are provided as SEQ ID NOs: 123-140.

Non-native gene coding sequences flanked by the complement of the viral polymerase binding site/promoter, e.g., the complement of 3′-hMPV virus terminus, or the complements of both the 3′- and 5′-hMPV virus termini may be constructed using techniques known in the art. In more specific embodiments, a recombinant virus of the invention contains the leader and trailer sequence of hMPV or APV. In certain embodiments, the intergenic regions are obtained from hMPV or APV. The resulting RNA templates may be of the negative-polarity and contain appropriate terminal sequences which enable the viral RNA-synthesizing apparatus to recognize the template. Alternatively, positive-polarity RNA templates which contain appropriate terminal sequences which enable the viral RNA-synthesizing apparatus to recognize the template, may also be used. Recombinant DNA molecules containing these hybrid sequences can be cloned and transcribed by a DNA-directed RNA polymerase, such as bacteriophage T7, T3, the SP6 polymerase or eukaryotic polymerase such as polymerase I and the like, to produce in vitro or in vivo the recombinant RNA templates which possess the appropriate viral sequences that allow for viral polymerase recognition and activity. In a more specific embodiment, the RNA polymerase is fowlpox virus T7 RNA polymerase or a MVA T7 RNA polymerase.

An illustrative approach for constructing these hybrid molecules is to insert the non-native nucleotide sequence into a DNA complement of an hMPV, APV, APV/hMPV or hMPV/APV genome, so that the heterologous sequence is flanked by the viral sequences required for viral polymerase activity; i.e., the viral polymerase binding site/promoter, hereinafter referred to as the viral polymerase binding site, and a polyadenylation site. In a preferred embodiment, the heterologous coding sequence is flanked by the viral sequences that comprise the replication promoters of the 5′ and 3′ termini, the gene start and gene end sequences, and the packaging signals that are found in the 5′ and/or the 3′ termini. In an alternative approach, oligonucleotides encoding the viral polymerase binding site, e.g., the complement of the 3′-terminus or both termini of the virus genomic segment can be ligated to the heterologous coding sequence to construct the hybrid molecule.

Suitable restriction enzyme sites can readily be placed anywhere within a viral cDNA through the use of site-directed mutagenesis (e.g., see, for example, the techniques described by Kunkel, 1985, Proc. Natl. Acad. Sci. U.S.A. 82; 488). Variations in polymerase chain reaction (PCR) technology also allow for the specific insertion of sequences (i.e., restriction enzyme sites) and allow for the facile construction of hybrid molecules. Alternatively, PCR reactions could be used to prepare recombinant templates without the need of cloning. For example, PCR reactions could be used to prepare double-stranded DNA molecules containing a DNA-directed RNA polymerase promoter (e.g., bacteriophage T3, T7 or SP6) and the hybrid sequence containing the heterologous gene and the hMPV polymerase binding site. RNA templates could then be transcribed directly from this recombinant DNA. In yet another embodiment, the recombinant RNA templates may be prepared by ligating RNAs specifying the negative polarity of the heterologous gene and the viral polymerase binding site using an RNA ligase.

In addition, one or more nucleotides can be added in the untranslated region to adhere to the “Rule of Six” which may be important in obtaining virus rescue. The “Rule of Six” applies to many paramyxoviruses and states that the RNA nucleotide genome must be divisible by six to be functional. The addition of nucleotides can be accomplished by techniques known in the art such as using a commercial mutagenesis kits such as the QuikChange mutagenesis kit (Stratagene). After addition of the appropriate number of nucleotides, the correct DNA fragment can then be isolated by digestion with appropriate restriction enzyme and gel purification. Sequence requirements for viral polymerase activity and constructs which may be used in accordance with the invention are described in the subsections below.

In certain embodiments, the leader and or trailer sequence of the virus are modified relative to the wild type virus. In certain more specific embodiments, the lengths of the leader and/or trailer are altered. In other embodiments, the sequence(s) of the leader and/or trailer are mutated relative to the wild type virus.

The production of a recombinant virus of the invention relies on the replication of a partial or full-length copy of the negative sense viral RNA (vRNA) genome or a complementary copy thereof (cRNA). This vRNA or cRNA can be isolated from infectious virus, produced upon in-vitro transcription, or produced in cells upon transfection of nucleic acids. Second, the production of recombinant negative strand virus relies on a functional polymerase complex. Typically, the polymerase complex of pneumoviruses consists of N, P, L and possibly M2 proteins, but is not necessarily limited thereto.

Polymerase complexes or components thereof can be isolated from virus particles, isolated from cells expressing one or more of the components, or produced upon transfection of specific expression vectors.

Infectious copies of MPV can be obtained when the above mentioned vRNA, CRNA, or vectors expressing these RNAs are replicated by the above mentioned polymerase complex 16 (Schnell et al., 1994, EMBO J. 13: 4195-4203; Collins, et al., 1995, PNAS 92: 11563-11567; Hoffmann, et al., 2000, PNAS 97: 6108-6113; Bridgen, et al., 1996, PNAS 93: 15400-15404; Palese, et al., 1996, PNAS 93: 11354-11358; Peeters, et al., 1999, J. Virol. 73: 5001-5009; Durbin, et al., 1997, Virology 235: 323-332).

The invention also provides a host cell comprising a nucleic acid or a vector according to the invention. Plasmid or viral vectors containing the polymerase components of MPV (N, P, L and M2, but not necessarily limited thereto) are generated in prokaryotic cells for the expression of the components in relevant cell types. Plasmid or viral vectors containing full-length or partial copies of the MPV genome will be generated in prokaryotic cells for the expression of viral nucleic acids in-vitro or in-vivo.

In addition, eukaryotic cells, transiently or stably expressing one or more full-length or partial MPV proteins can be used. Such cells can be made by transfection (proteins or nucleic acid vectors), infection (viral vectors) or transduction (viral vectors) and may be useful for complementation of mentioned wild type, attenuated, replication-defective or chimeric viruses.

Bicistronic mRNA could be constructed to permit internal initiation of translation of viral sequences and allow for the expression of foreign protein coding sequences from the regular terminal initiation site. Alternatively, a bicistronic mRNA sequence may be constructed wherein the viral sequence is translated from the regular terminal open reading frame, while the foreign sequence is initiated from an internal site. Certain internal ribosome entry site (IRES) sequences may be utilized. The IRES sequences which are chosen should be short enough to not interfere with MPV packaging limitations. Thus, it is preferable that the IRES chosen for such a bicistronic approach be no more than 500 nucleotides in length. In a specific embodiment, the IRES is derived from a picornavirus and does not include any additional picornaviral sequences. Specific IRES elements include, but are not limited to the mammalian BiP IRES and the hepatitis C virus IRES.

Alternatively, a foreign protein may be expressed from a new internal transcriptional unit in which the transcriptional unit has an initiation site and polyadenylation site. In another embodiment, the foreign gene is inserted into a MPV gene such that the resulting expressed protein is a fusion protein.

In some embodiments, the cDNA encoding the mMPV encodes the wild-type leader sequence of the virus. In certain embodiments, the cDNA encoding the mMPV encodes the C4A mutation in the leader sequence of the virus, i.e., a nucleotide substitution at position 4 of the leader sequence that results in an A in place of the C.

### 5.6 Rescue of Recombinant Virus Particles

Any technique known to those of skill in the art may be used to achieve replication and rescue of recombinant and chimeric viruses. Descriptions of mMPV rescue can be found in International Patent Application No PCT/US03/05271 (published as WO 03/072719; see Section 5.6) and International Patent Application No. PCT/US04/12724 (published as WO 04/096993; see Section 5.6), both of which are incorporated herein by reference in their entireties.

In order to prepare the chimeric and recombinant viruses of the invention, a cDNA encoding the genome of a recombinant or chimeric virus of the invention in the plus or minus sense (i.e., the genome or the antigenome) may be used to transfect a host cell which provide viral proteins and functions required for replication and rescue. Alternatively, cells may be transfected with helper virus before, during, or after transfection by the DNA or RNA molecule coding for the recombinant virus of the invention. The synthetic recombinant plasmid DNAs and RNAs of the invention can be replicated and rescued into infectious virus particles by any number of techniques known in the art, as described, e.g., in U.S. Pat. No. 5,166,057 issued Nov. 24, 1992; in U.S. Pat. No. 5,854,037 issued Dec. 29, 1998; in European Patent Publication EP 0702085A1, published Feb. 20, 1996; in U.S. patent application Ser. No. 09/152,845; in International Patent Publications PCT WO97/12032 published Apr. 3, 1997; WO96/34625 published Nov. 7, 1996; in European Patent Publication EP-A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 47SA1 published Jun. 25, 1997, each of which is incorporated by reference herein in its entirety.

In one embodiment, of the present invention, synthetic recombinant viral RNAs may be prepared that contain the non-coding regions (leader and trailer) of the negative strand virus RNA which are essential for the recognition by viral polymerases and for packaging signals necessary to generate a mature virion. There are a number of different approaches which may be used to apply the reverse genetics approach to rescue negative strand RNA viruses. First, the recombinant RNAs are synthesized from a recombinant DNA template and reconstituted in vitro with purified viral polymerase complex to form recombinant ribonucleoproteins (RNPs) which can be used to transfect cells. In another approach, a more efficient transfection is achieved if the viral polymerase proteins are present during transcription of the synthetic RNAs either in vitro or in vivo. With this approach the synthetic RNAs may be transcribed from cDNA plasmids which are either co-transcribed in vitro with cDNA plasmids encoding the polymerase proteins, or transcribed in vivo in the presence of polymerase proteins, i.e., in cells which transiently or constitutively express the polymerase proteins.

In additional approaches described herein, infectious chimeric or recombinant virus may be replicated in host cell systems that express a metapneumoviral polymerase protein (e.g., in virus/host cell expression systems; transformed cell lines engineered to express a polymerase protein, etc.), so that infectious chimeric or recombinant virus are replicated and rescued. In this instance, helper virus need not be utilized since this function is provided by the viral polymerase proteins expressed.

One approach involves supplying viral proteins and functions required for replication in vitro prior to transfecting host cells. In such an embodiment, viral proteins may be supplied in the form of wildtype virus, helper virus, purified viral proteins or recombinantly expressed viral proteins. The viral proteins may be supplied prior to, during or post transcription of the synthetic cDNAs or RNAs encoding the chimeric virus. The entire mixture may be used to transfect host cells. In another approach, viral proteins and functions required for replication may be supplied prior to or during transcription of the synthetic cDNAs or RNAs encoding the chimeric virus. In such an embodiment, viral proteins and functions required for replication are supplied in.the form of wildtype virus, helper virus, viral extracts, synthetic cDNAs or RNAs which express the viral proteins are introduced into the host cell via infection or transfection. This infection/transfection takes place prior to or simultaneous to the introduction of the synthetic cDNAs or RNAs encoding the chimeric virus genome.

Helper viruses that may be used in accordance with the invention, include those that express the polymerase viral proteins natively, such as MPV or APV. Alternatively, helper viruses may be used that have been recombinantly engineered to provide the polymerase viral proteins

In certain aspects, the host cell expresses components of the viral polymerase constitutively. In other aspects, the expression of the viral polymerase components is induced. In certain aspects, the host cell is transiently transfected with the plasmids encoding the viral polymerase components. In other aspects, the host cell is a stable cell line with the nucleotide sequences encoding the viral polymerase components. In other embodiments, the host cell is infected with a helper virus that provides the RNA polymerase.

In yet another embodiment, viral proteins and functions required for replication may be supplied as genetic material in the form of synthetic cDNAs or RNAs so that they are co-transcribed with the synthetic cDNAs or RNAs encoding the chimeric virus. Plasmids that encode express the virus and the viral polymerase and/or other viral functions are co-transfected into host cells. Alternatively, rescue of the recombinant viruses of the invention may be accomplished by the use of Modified Vaccinia Virus Ankara (MVA) encoding T7 RNA polymerase, or a combination of MVA and plasmids encoding the polymerase proteins (N, P, and L). For example, MVA-T7 or Fowl Pox-T7 can be infected into Vero cells, LLC-MK-2 cells, HEp-2 cells, LF 1043 (HEL) cells, tMK cells, LLC-MK2, HUT 292, FRHL-2 (rhesus), FCL-1 (green monkey), WI-38 (human), MRC-5 (human) cells, 293 T cells, QT 6 cells, QT 35 cells and CEF cells. After infection with MVA-T7 or Fowl Pox-T7, a full length antigenomic or genomic cDNA encoding the recombinant virus of the invention may be transfected into the cells together with the N, P, L, and M2.1 encoding expression plasmids. Alternatively, the polymerase may be provided by plasmid transfection. The cells and cell supernatant can subsequently be harvested and subjected to a single freeze-thaw cycle. The resulting cell lysate may then be used to infect a fresh Vero cell monolayer in the presence of 1-beta-D-arabinofuranosylcytosine (ara C), a replication inhibitor of vaccinia virus, to generate a virus stock. The supernatant and cells from these plates can then be harvested, freeze-thawed once and the presence of recombinant virus particles of the invention can be assayed by immunostaining of virus plaques using antiserum specific to the particular virus.

Another approach to propagating the chimeric or recombinant virus may involve co-cultivation with wild-type virus. This could be done by simply taking recombinant virus and co-infecting cells with this and another wild-type virus. The wild-type virus should complement for the defective virus gene product and allow growth of both the wild-type and recombinant virus.

In order to achieve replication and packaging of the viral genome, it is important that the leader and trailer sequences retain the signals necessary for viral polymerase recognition. The leader and trailer sequences for the viral RNA genome can be optimized or varied to improve and enhance viral replication and rescue. Alternatively, the leader and trailer sequences can be modified to decrease the efficiency of viral replication and packaging, resulting in a rescued virus with an attenuated phenotype. Examples of different leader and trailer sequences, include, but are not limited to, leader and trailer sequences of a paramyxovirus. In a specific embodiment of the invention, the leader and trailer sequence is that of a wild type or mutated hMPV. In another embodiment of the invention, the leader and trailer sequence is that of a PIV, APV, or an RSV. In yet another embodiment of the invention, the leader and trailer sequence is that of a combination of different virus origins. By way of example and not meant to limit the possible combination, the leader and trailer sequence can be a combination of any of the leader and trailer sequences of hMPV, PIV, APV, RSV, or any other paramyxovirus. Examples of modifications to the leader and trailer sequences include varying the spacing relative to the viral promoter, varying the sequence, e.g., varying the number of G residues (typically 0 to 3), and defining the 5′ or 3′ end using ribozyme sequences, including, Hepatitis Delta Virus (HDV) ribozyme sequence, Hammerhead ribozyme sequences, or fragments thereof, which retain the ribozyme catalytic activity, and using restriction enzymes for run-off RNA produced in vitro.

In an alternative embodiment, the efficiency of viral replication and rescue may be enhanced if the viral genome is of hexamer length. In order to ensure that the viral genome is of the appropriate length, the 5′ or 3′ end may be defined using ribozyme sequences, including, Hepatitis Delta Virus (HDV) ribozyme sequence, Hammerhead ribozyme sequences, or fragments thereof, which retain the ribozyme catalytic activity, and using restriction enzymes for run-off RNA produced in vitro.

In order for the genetic material encoding the viral genome and for the genetic material encoding the RNA polymerase components to be transcribed, the genetic material is engineered to be placed under the control of appropriate transcriptional regulatory sequences, e.g., promoter sequences recognized by a polymerase. In preferred embodiments, the promoter sequences are recognized by a T7, Sp6 or T3 polymerase. In yet another embodiment, the promoter sequences are recognized by cellular DNA dependent RNA polymerases, such as RNA polymerase I (Pol I) or RNA polymerase II (Pol II). The genetic material encoding the viral genome may be placed under the control of the transcriptional regulatory sequences, so that either a positive or negative strand copy of the viral genome is transcribed. The genetic material encoding the viral genome is recombinantly engineered to be operatively linked to the transcriptional regulatory sequences in the context of an expression vector, such as a plasmid based vector, e.g. a plasmid with a pol II promoter such as the immediate early promoter of CMV, a plasmid with a T7 promoter, or a viral based vector, e.g., pox viral vectors, including vaccinia vectors, MVA-T7, and Fowl pox vectors.

The genetic material encoding the viral genome may be modified to enhance expression by the polymerase of choice, e.g., varying the number of G residues (typically 0 to 3) upstream of the leader or trailer sequences to optimize expression from a T7 promoter. Replication and packaging of the viral genome occurs intracellularly in a host cell permissive for viral replication and packaging. There are a number of methods by which the host cell can be engineered to provide sufficient levels of the viral polymerase and structural proteins necessary for replication and packaging, including, host cells infected with an appropriate helper virus, host cells engineered to stably or constitutively express the viral polymerase and structural proteins, or host cells engineered to transiently or inducibly express the viral polymerase and structural proteins.

Protein function required for MPV viral replication includes, but not limited to, the polymerase proteins P, N, L, and M2.1.

In order to achieve efficient viral replication and packaging, high levels of expression of the polymerase proteins is preferred. Such levels are obtained using 100-200 ng L/pCITE, 200-400 ng N/pCITE, 200-400 ng P/pCITE, and 100-200 ng M2.1/pCITE plasmids encoding paramyxovirus proteins together with 2-4 ug of plasmid encoding the full-length viral cDNA transfected into cells infected with MVA-T7. In another embodiment, 0.1-2.0 μg of pSH25 (CAT expressing), 0.1-3.0 μg of pRF542 (expressing T7 polymerase), 0.1-0.8 μg pCITE vector with N cDNA insert, and 0.1-1.0 μg of each of three pCITE vectors containing P, L and M2.1 cDNA insert are used. Alternatively, one or more polymerase and structural proteins can be introduced into the cells in conjunction with the genetic material by transfecting cells with purified ribonucleoproteins. Host cells that are permissive for MPV viral replication and packaging are preferred. Examples of preferred host cells include, but are not limited to, 293T, Vero, tMK, and BHK. Other examples of host cells include, but are not limited to, LLC-MK-2 cells, Hep-2 cells, LF 1043 (HEL) cells, LLC-MK2, HUT 292, FRHL-2 (rhesus), FCL-1 (green monkey), WI-38 (human), MRC-5 (human) cells, QT 6 cells, QT 35 cells and CEF cells.

In alternative embodiments of the invention, the host cells can be treated using a number of methods in order to enhance the level of transfection and/or infection efficiencies, protein expression, in order to optimize viral replication and packaging. Such treatment methods, include, but are not limited to, sonication, freeze/thaw, and heat shock. Furthermore, standard techniques known to the skilled artisan can be used to optimize the transfection and/or infection protocol, including, but are not limited to, DEAE-dextran-mediated transfection, calcium phosphate precipitation, lipofectin treatment, liposome-mediated transfection and electroporation. The skilled artisan would also be familiar with standard techniques available for the optimization of transfection/infection protocols. By way of example, and not meant to limit the available techniques, methods that can be used include, manipulating the timing of infection relative to transfection when a virus is used to provide a necessary protein, manipulating the timing of transfections of different plasmids, and affecting the relative amounts of viruses and transfected plasmids.

The viruses of the invention can be propagated using any technique known to the skilled artisan. In a particular embodiment, the viruses are propagated in serum-free medium as described in International Patent Application No. PCT/US04/12724 (published as WO 04/096993; Section 5.6).

### 5.7 Additional Attenuating Mutations

In certain embodiments of the invention additional mutations can be introduced into the mutant mMPV, such as hMPV. In certain embodiments, the additional mutations contribute to an attenuated phenotype of the viruses of the invention. In certain embodiments, the additional mutation is a deletion of an entire open reading frame, or a deletion that reduces the function of the affected gene. For example, the additional mutation can be a deletion of (or in) the M2.2 gene or the SH gene.

In particular, the recombinant viruses of the invention exhibit an attenuated phenotype in a subject to which the virus is administered as a vaccine. Attenuation can be achieved by any method known to a skilled artisan. Without being bound by theory, the attenuated phenotype of the recombinant virus can be caused, e.g., by using a virus that naturally does not replicate well in an intended host (e.g., using an APV in human), by reduced replication of the viral genome, by reduced ability of the virus to infect a host cell, or by reduced ability of the viral proteins to assemble to an infectious viral particle relative to the wild type strain of the virus. The viability of certain sequences of the virus, such as the leader and the trailer sequence can be tested using a minigenome assay (see section 5.8).

The attenuated phenotypes of a recombinant virus of the invention can be tested by any method known to the artisan (see, e.g., section 5.8). A candidate virus can, for example, be tested for its ability to infect a host or for the rate of replication in a cell culture system. In certain embodiments, a mimi-genome system is used to test the attenuated virus when the gene that is altered is N, P, L, M2, F, G, M2.1, M2.2 or a combination thereof. In certain embodiments, growth curves at different temperatures are used to test the attenuated phenotype of the virus. For example, an attenuated virus is able to grow at 35° C., but not at 39° C. or 40° C. In certain embodiments, different cell lines can be used to evaluate the attenuated phenotype of the virus. For example, an attenuated virus may only be able to grow in monkey cell lines but not the human cell lines, or the achievable virus titers in different cell lines are different for the attenuated virus. In certain embodiments, viral replication in the respiratory tract of a small animal model, including but not limited to, hamsters, cotton rats, mice and guinea pigs, is used to evaluate the attenuated phenotypes of the virus. In other embodiments, the immune response induced by the virus, including but not limited to, the antibody titers (e.g., assayed by plaque reduction neutralization assay or ELISA) is used to evaluate the attenuated phenotypes of the virus. In a specific embodiment, the plaque reduction neutralization assay or ELISA is carried out at a low dose. In certain embodiments, the ability of the recombinant virus to elicit pathological symptoms in an animal model can be tested. A reduced ability of the virus to elicit pathological symptoms in an animal model system is indicative of its attenuated phenotype. In a specific embodiment, the candidate viruses are tested in a monkey model for nasal infection, indicated by mucous production.

The viruses of the invention can be attenuated such that one or more of the functional characteristics of the virus are impaired. In certain embodiments, attenuation is measured in comparison to the wild type strain of the virus from which the attenuated virus is derived. In other embodiments, attenuation is determined by comparing the growth of an attenuated virus in different host systems. Thus, for a non-limiting example, an APV is said to be attenuated when grown in a human host if the growth of the APV in the human host is reduced compared to the growth of the APV in an avian host.

In certain embodiments, the attenuated virus of the invention is capable of infecting a host, is capable of replicating in a host such that infectious viral particles are produced. In comparison to the wild type strain, however, the attenuated strain grows to lower titers or grows more slowly. Any technique known to the skilled artisan can be used to determine the growth curve of the attenuated virus and compare it to the growth curve of the wild type virus. For exemplary methods see Example section, infra. In a specific embodiment, the attenuated virus grows to a titer of less than 10⁵ pfu/ml, of less than 10⁴ pfu/ml, of less than 10³ pfu/ml, or of less than 10² pfu/ml in Vero cells.

In certain embodiments, the attenuated hMPV of the invention cannot replicate in human cells as well as the wild type virus (e.g., wild type mammalian MPV) does. However, the attenuated virus can replicate well in a cell line that lack interferon functions, such as Vero cells.

In other embodiments, the attenuated virus of the invention is capable of infecting a host, of replicating in the host, and of causing proteins of the virus of the invention to be inserted into the cytoplasmic membrane, but the attenuated virus does not cause the host to produce new infectious viral particles. In certain embodiments, the attenuated virus infects the host, replicates in the host, and causes viral proteins to be inserted in the cytoplasmic membrane of the host with the same efficiency as the wild type mammalian virus. In other embodiments, the ability of the attenuated virus to cause viral proteins to be inserted into the cytoplasmic membrane into the host cell is reduced compared to the wild type virus. In certain embodiments, the ability of the attenuated mammalian virus to replicate in the host is reduced compared to the wild type virus. Any technique known to the skilled artisan can be used to determine whether a virus is capable of infecting a mammalian cell, of replicating within the host, and of causing viral proteins to be inserted into the cytoplasmic membrane of the host. For illustrative methods see section 5.8.

In certain embodiments, the attenuated virus of the invention is capable of infecting a host. In contrast to the wild type mammalian MPV, however, the attenuated mammalian MPV cannot be replicated in the host. In a specific embodiment, the attenuated mammalian virus can infect a host and can cause the host to insert viral proteins in its cytoplasmic membranes, but the attenuated virus is incapable of being replicated in the host. Any method known to the skilled artisan can be used to test whether the attenuated mammalian MPV has infected the host and has caused the host to insert viral proteins in its cytoplasmic membranes.

In certain embodiments, the ability of the attenuated mammalian virus to infect a host is reduced compared to the ability of the wild type virus to infect the same host. Any technique known to the skilled artisan can be used to determine whether a virus is capable of infecting a host. For illustrative methods see section 5.8.

In certain embodiments, mutations (e.g., missense mutations) are introduced into the genome of the virus to generated a virus with an attenuated phenotype. Mutations (e.g., missense mutations) can be introduced into the N-gene, the P-gene, the M-gene, the F-gene, the M2-gene, the SH-gene, the G-gene or the L-gene of the recombinant virus. Mutations can be additions, substitutions, deletions, or combinations thereof. In specific embodiments, a single amino acid deletion mutation for the N, P, L, F, G, M2.1, M2.2 or M2 proteins is introduced, which can be screened for functionality in the mini-genome assay system and be evaluated for predicted functionality in the virus. In more specific embodiments, the missense mutation is a cold-sensitive mutation. In other embodiments, the missense mutation is a heat-sensitive mutation. In one embodiment, major phosphorylation sites of P protein of the virus is removed. In another embodiment, a mutation or mutations are introduced into the L gene of the virus to generate a temperature sensitive strain. In yet another embodiment, the cleavage site of the F gene is mutated in such a way that cleavage does not occur or occurs at very low efficiency. In certain, more specific embodiments, the motif with the amino acid sequence RQSR at amino acid postions 99 to 102 of the F protein of hMPV is mutated. A mutation can be, but is not limited to, a deletion of one or more amino acids, an addition of one or more amino acids, a substitution (conserved or non-conserved) of one or more amino acids or a combination thereof. In some strains of hMPV, the cleavage site is RQPR (see Example “PIOIS”). In certain embodiments, the cleavage site with the amino acid sequence is RQPR is mutated. In more specific embodiments, the cleavage site of the F protein of hMPV is mutated such that the infectivity of hMPV is reduced. In certain embodiments, the infectivity of hMPV is reduced by a factor of at least 5, 10, 50, 100, 500, 10, 5×10³, 10⁴, 5×10⁴, 10⁵, 5×10⁵, or at least 10⁶. In certain embodiments, the infectivity of hMPV is reduced by a factor of at most 5, 10, 50, 100, 500, 10³, 5×10³, 10⁴, 5×10⁴, 10⁵, 5×10⁵, or at most 10⁶.

In other embodiments, deletions are introduced into the genome of the recombinant virus. In more specific embodiments, a deletion can be introduced into the N-gene, the P-gene, the M-gene, the F-gene, the M2-gene, the SH-gene, the G-gene or the L-gene of the recombinant virus. In specific embodiments, the deletion is in the M2-gene of the recombinant virus of the present invention. In other specific embodiments, the deletion is in the SH-gene of the recombinant virus of the present invention. In yet another specific embodiment, both the M2-gene and the SH-gene are deleted.

In certain embodiments, the intergenic region of the recombinant virus is altered. In one embodiment, the length of the intergenic region is altered. In another embodiment, the intergenic regions are shuffled from 5′ to 3′ end of the viral genome.

In other embodiments, the genome position of a gene or genes of the recombinant virus is changed. In one embodiment, the F or G gene is moved to the 3′ end of the genome. In another embodiment, the N gene is moved to the 5′ end of the genome.

In certain embodiments, attenuation of the virus is achieved by replacing a gene of the wild type virus with the analogous gene of a virus of a different species (e.g., of RSV, APV, PIV3 or mouse pneumovirus), of a different subgroup, or of a different variant. In illustrative embodiments, the N-gene, the P-gene, the M-gene, the F-gene, the M2-gene, the SH-gene, the G-gene or the L-gene of a mammalian MPV is replaced with the N-gene, the P-gene, the M-gene, the F-gene, the M2-gene, the SH-gene, the G-gene or the L-gene, respectively, of an APV. In other illustrative embodiments, the N-gene, the P-gene, the M-gene, the F-gene, the M2-gene, the SH-gene, the G-gene or the L-gene of APV is replaced with the N-gene, the P-gene, the M-gene, the F-gene, the M2-gene, the SH-gene, the G-gene or the L-gene, respectively, of a mammalian MPV. In a preferred embodiment, attenuation of the virus is achieved by replacing one or more polymerase associated genes (e.g., N, P, L or M2) with genes of a virus of a different species.

In certain embodiments, attenuation of the virus is achieved by replacing one or more specific domains of a protein of the wild type virus with domains derived from the corresponding protein of a virus of a different species. In an illustrative embodiment, the ectodomain of a F protein of APV is replaced with an ectodomain of a F protein of a mammalian MPV. In a preferred embodiment, one or more specific domains of L, N, or P protein are replaced with domains derived from corresponding proteins of a virus of a different species. In certain other embodiments, attenuation of the virus is achieved by deleting one or more specific domains of a protein of the wild type virus. In a specific embodiment, the transmembrane domain of the F-protein is deleted.

In certain embodiments of the invention, the leader and/or trailer sequence of the recombinant virus of the invention can be modified to achieve an attenuated phenotype. In certain, more specific embodiments, the leader and/or trailer sequence is reduced in length relative to the wild type virus by at least 1 nucleotide, at least 2 nucleotides, at least 3 nucleotides, at least 4 nucleotides, at least 5 nucleotides or at least 6 nucleotides. In certain other, more specific embodiments, the sequence of the leader and/or trailer of the recombinant virus is mutated. In a specific embodiment, the leader and the trailer sequence are 100% complementary to each other. In other embodiments, 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides are not complementary to each other where the remaining nucleotides of the leader and the trailer sequences are complementary to each other. In certain embodiments, the non-complementary nucleotides are identical to each other. In certain other embodiments, the non-complementary nucleotides are different from each other. In other embodiments, if the non-complementary nucleotide in the trailer is purine, the corresponding nucleotide in the leader sequence is also a purine. In other embodiments, if the non-complementary nucleotide in the trailer is pyrimidine, the corresponding nucleotide in the leader sequence is also a purine. In certain embodiments of the invention, the leader and/or trailer sequence of the recombinant virus of the invention can be replaced with the leader and/or trailer sequence of a another virus, e.g., with the leader and/or trailer sequence of RSV, APV, PIV3, mouse pneumovirus, or with the leader and/or trailer sequence of a human metapneumovirus of a subgroup or variant different from the hMPV metapneumovirus from which the protein-encoding parts of the recombinant virus are derived.

When a live attenuated vaccine is used, its safety must also be considered. Preferably the vaccine does not cause disease. Any techniques known in the art that can make a vaccine safe may be used in the present invention. In addition to attenuation techniques, other techniques may be used. One non-limiting example is to use a soluble heterologous gene that cannot be incorporated into the virion membrane. For example, a single copy of the soluble RSV F gene, a version of the RSV gene lacking the transmembrane and cytosolic domains, can be used. Since it cannot be incorporated into the virion membrane, the virus tropism is not expected to change.

Various assays can be used to test the safety of a vaccine. See section 5.8, infra. Particularly, sucrose gradients and neutralization assays can be used to test the safety. A sucrose gradient assay can be used to determine whether a heterologous protein is inserted in a virion. If the heterologous protein is inserted in the virion, the virion should be tested for its ability to cause symptoms even if the parental strain does not cause symptoms. Without being bound by theory, if the heterologous protein is incorporated in the virion, the virus may have acquired new, possibly pathological, properties.

In certain embodiments, one or more genes are deleted from the hMPV genome to generate an attenuated virus. In more specific embodiments, the M2.2 ORF, the M2.1 ORF, the M2 gene, the SH gene and/or the G2 gene is deleted.

In other embodiments, small single amino acid deletions are introduced in genes involved in virus replication to generate an attenuated virus. In more specific embodiments, a small single amino acid deletion is introduced in the N, L, or the P gene. In certain specific embodiments, one or more of the following amino acids are mutated in the L gene of a recombinant hMPV: Phe at amino acid position 456, Glu at amino acid position 749, Tyr at amino acid position 1246, Met at amino acid position 1094 and Lys at amino acid position 746 to generate an attenuated virus. A mutation can be, e.g., a deletion or a substitution of an amino acid. An amino acid substitution can be a conserved amino acid substitution or a non-conserved amino acid substitution. Illustrative examples for conserved amino acid exchanges are amino acid substitutions that maintain structural and/or functional properties of the amino acids' side-chains, e.g., an aromatic amino acid is substituted for another aromatic amino acid, an acidic amino acid is substituted for another acidic amino acid, a basic amino acid is substituted for another basic amino acid, and an aliphatic amino acid is substituted for another aliphatic amino acid. In contrast, examples of non-conserved amino acid exchanges are amino acid substitutions that do not maintain structural and/or functional properties of the amino acids' side-chains, e.g., an aromatic amino acid is substituted for a basic, acidic, or aliphatic amino acid, an acidic amino acid is substituted for an aromatic, basic, or aliphatic amino acid, a basic amino acid is substituted for an acidic, aromatic or aliphatic amino acid, and an aliphatic amino acid is substituted for an aromatic, acidic or basic amino acid. In even more specific embodiments Phe at amino acid position 456 is replaced by a Leu.

In certain embodiments, one nucleic acid is substituted to encode one amino acid exchange. In other embodiments, two or three nucleic acids are substituted to encode one amino acid exchange. It is preferred that two or three nucleic acids are substituted to reduce the risk of reversion to the wild type protein sequence.

In other embodiments, small single amino acid deletions are introduced in genes involved in virus assembly to generate an attenuated virus. In more specific embodiments, a small single amino acid deletion is introduced in the M gene or the M2 gene. In a preferred embodiment, the M gene is mutated.

In even other embodiments, the gene order in the genome of the virus is changed from the gene order of the wild type virus to generate an attenuated virus. In a more specific embodiment, the F, SH, and/or the G gene is moved to the 3′ end of the viral genome. In another embodiment, the N gene is moved to the 5′ end of the viral genome.

In other embodiments, one or more gene start sites are mutated or substituted with the analogous gene start sites of another virus (e.g., RSV, PIV3, APV or mouse pneumovirus) or of a human metapneumovirus of a subgroup or a variant different from the human metapneumovirus from which the protein-encoding parts of the recombinant virus are derived. In more specific embodiments, the gene start site of the N-gene, the P-gene, the M-gene, the F-gene, the M2-gene, the SH-gene, the G-gene and/or the L-gene is mutated or replaced with the start site of the N-gene, the P-gene, the M-gene, the F-gene, the M2-gene, the SH-gene, the G-gene and/or the L-gene, respectively, of another virus (e.g., RSV, PIV3, APV or mouse pneumovirus) or of a human metapneumovirus of a subgroup or a variant different from the human metapneumovirus from which the protein-encoding parts of the recombinant virus are derived.

In certain embodiments of the invention, attenuation is achieved by replacing one or more of the genes of a virus with the analogous gene of a different virus, different strain, or different viral isolate. In certain embodiments, one or more of the genes of a metapneumovirus, such as a mammalian metapneumovirus, e.g., hMPV, or APV, is replaced with the analogous gene(s) of another paramyxovirus. In a more specific embodiment, the N-gene, the P-gene, the M-gene, the F-gene, the M2-gene, the M2.1 ORF, the M2.2 ORF, the SH-gene, the G-gene or the L-gene or any combination of two or more of these genes of a mammalian metapneumovirus, e.g., hMPV, is replaced with the analogous gene of another viral species, strain or isolate, wherein the other viral species can be, but is not limited to, another mammalian metapneumovirus, APV, or RSV.

In more specific embodiments, one or more of the genes of human metapneumovirus are replaced with the analogous gene(s) of another isolate of human metapneumovirus. E.g., the N-gene, the P-gene, the M-gene, the F-gene, the M2-gene, the M2.1 ORF, the M2.2 ORF, the SH-gene, the G-gene or the L-gene or any combination of two or more of these genes of isolate NL/1/99, NL/1/00, NL/17/00, or NL/1/94 is replaced with the analogous gene or combination of genes, i.e., the N-gene, the P-gene, the M-gene, the F-gene, the M2-gene, the M2.1 ORF, the M2.2 ORF, the SH-gene, the G-gene or the L-gene, of a different isolate, e.g., NL/1/99, NL/1/00, NL/17/00, or NL/1/94.

In certain embodiments, one or more regions of the genome of a virus is/are replaced with the analogous region(s) from the genome of a different viral species, strain or isolate. In certain embodiments, the region is a region in a coding region of the viral genome. In other embodiments, the region is a region in a non-coding region of the viral genome. In certain embodiments, two regions of two viruses are analogous to each other if the two regions support the same or a similar function in the two viruses. In certain other embodiments, two regions of two viruses are analogous if the two regions provide the same of a similar structural element in the two viruses. In more specific embodiments, two regions are analogous if they encode analogous protein domains in the two viruses, wherein analogous protein domains are domains that have the same or a similar function and/or structure.

In certain embodiments, one or more of regions of a genome of a metapneumovirus, such as a mammalian metapneumovirus, e.g., hMPV, or APV, is/are replaced with the analogous region(s) of the genome of another paramyxovirus. In certain embodiments, one or more of regions of the genome of a paramyxovirus is/are replaced with the analogous region(s) of the genome of a mammalian metapneumovirus, e.g., hMPV, or APV. In more specific embodiments, a region of the N-gene, the P-gene, the M-gene, the F-gene, the M2-gene, the M2.1 ORF, the M2.2 ORF, the SH-gene, the G-gene or the L-gene or any combination of two or more regions of these genes of a mammalian metapneumovirus, e.g., hMPV, is replaced with the analogous region of another viral species, strain or isolate. Another viral species can be, but is not limited to, another mammalian metapneumovirus, APV, or RSV.

In more specific embodiments, one or more regions of human metapneumovirus are replaced with the analogous region(s) of another isolate of human metapneumovirus. E.g., one or more region(s) of the N-gene, the P-gene, the M-gene, the F-gene, the M2-gene, the M2.1 ORF, the M2.2 ORF, the SH-gene, the G-gene or the L-gene or any combination of two or more regions of isolate NL/1/99, NL/1/00, NL/17/00, or NL/1/94 is replaced with the analogous region(s) of a different isolate of hMPV, e.g., NL/1/99, NL/1/00, NL/17/00, or NL/1/94.

In certain embodiments, the region is at least 5 nucleotides (nt) in length, at least 10 nt, at least 25 nt, at least 50 nt, at least 75 nt, at least 100 nt, at least 250 nt, at least 500 nt, at least 750 nt, at least 1 kb, at least 1.5 kb, at least 2 kb, at least 2.5 kb, at least 3 kb, at least 4 kb, or at least 5 kb in length. In certain embodiments, the region is at most 5 nucleotides (nt) in length, at most 10 nt, at most 25 nt, at most 50 nt, at most 75 nt, at most 100 nt, at most 250 nt, at most 500 nt, at most 750 nt, at most 1 kb, at most 1.5 kb, at most 2 kb, at most 2.5 kb, at most 3 kb, at most 4 kb, or at most 5 kb in length.

### 5.8 Assays for Use with the Invention

A number of assays may be employed in accordance with the present invention in order to determine the rate of growth of a chimeric or recombinant virus in a cell culture system, an animal model system or in a subject. A number of assays may also be employed in accordance with the present invention in order to determine the requirements of the chimeric and recombinant viruses to achieve infection, replication and packaging of virions.

Expression levels of non-native sequence in a chimeric virus of the invention can be determined by infecting cells in culture with a virus of the invention and subsequently measuring the level of protein expression by, e.g., Western blot analysis or ELISA using antibodies specific to the gene product of the heterologous sequence, or measuring the level of RNA expression by, e.g., Northern blot analysis using probes specific to the heterologous sequence. Similarly, expression levels of the heterologous sequence can be determined by infecting an animal model and measuring the level of protein expressed from the heterologous sequence of the recombinant virus of the invention in the animal model. The protein level can be measured by obtaining a tissue sample from the infected animal and then subjecting the tissue sample to Western blot analysis or ELISA, using antibodies specific to the gene product of the heterologous sequence. Further, if an animal model is used, the titer of antibodies produced by the animal against the gene product of the heterologous sequence can be determined by any technique known to the skilled artisan, including but not limited to, ELISA.

In certain embodiments, to facilitate the identification of the optimal position of the heterologous sequence in the viral genome and the optimal length of the intergenic region, the heterologous sequence encodes a reporter gene. Once the optimal parameters are determined, the reporter gene is replaced by a heterologous nucleotide sequence encoding an antigen of choice. Any reporter gene known to the skilled artisan can be used with the methods of the invention.

The rate of replication of the recombinant virus can be determined by any standard technique known to the skilled artisan. The rate of replication is represented by the growth rate of the virus and can be determined by plotting the viral titer over the time post infection. The viral titer can be measured by any technique known to the skilled artisan. In certain embodiments, a suspension containing the virus is incubated with cells that are susceptible to infection by the virus. Cell types that can be used with the methods of the invention include, but are not limited to, Vero cells, LLC-MK-2 cells, Hep-2 cells, LF 1043 (HEL) cells, MRC-5 cells, WI-38 cells, tMK cells, 293 T cells, QT 6 cells, QT 35 cells, or chicken embryo fibroblasts (CEF). Subsequent to the incubation of the virus with the cells, the number of infected cells is determined. In certain specific embodiments, the virus comprises a reporter gene. Thus, the number of cells expressing the reporter gene is representative of the number of infected cells. In a specific embodiment, the virus comprises a heterologous nucleotide sequence encoding for eGFP, and the number of cells expressing eGFP, i.e., the number of cells infected with the virus, is determined using FACS.

The assays described herein may be used to assay viral titre over time to determine the growth characteristics of the virus. In a specific embodiment, the viral titre is determined by obtaining a sample from the infected cells or the infected subject, preparing a serial dilution of the sample and infecting a monolayer of cells that are susceptible to infection with the virus at a dilution of the virus that allows for the emergence of single plaques. The plaques can then be counted and the viral titre express as plaque forming units per milliliter of sample. In a specific embodiment of the invention, the growth rate of a virus of the invention in a subject is estimated by the titer of antibodies against the virus in the subject. Without being bound by theory, the antibody titer in the subject reflects not only the viral titer in the subject but also the antigenicity. If the antigenicity of the virus is constant, the increase of the antibody titer in the subject can be used to determine the growth curve of the virus in the subject. In a preferred embodiment, the growth rate of the virus in animals or humans is best tested by sampling biological fluids of a host at multiple time points post-infection and measuring viral titer.

The expression of heterologous gene sequence in a cell culture system or in a subject can be determined by any technique known to the skilled artisan. In certain embodiments, the expression of the heterologous gene is measured by quantifying the level of the transcript. The level of the transcript can be measured by Northern blot analysis or by RT-PCR using probes or primers, respectively, that are specific for the transcript. The transcript can be distinguished from the genome of the virus because the virus is in the antisense orientation whereas the transcript is in the sense orientation. In certain embodiments, the expression of the heterologous gene is measured by quantifying the level of the protein product of the heterologous gene. The level of the protein can be measured by Western blot analysis using antibodies that are specific to the protein.

In a specific embodiment, the heterologous gene is tagged with a peptide tag. The peptide tag can be detected using antibodies against the peptide tag. The level of peptide tag detected is representative for the level of protein expressed from the heterologous gene. Alternatively, the protein expressed from the heterologous gene can be isolated by virtue of the peptide tag. The amount of the purified protein correlates with the expression level of the heterologous gene. Such peptide tags and methods for the isolation of proteins fused to such a peptide tag are well known in the art. A variety of peptide tags known in the art may be used in the modification of the heterologous gene, such as, but not limited to, the immunoglobulin constant regions, polyhistidine sequence (Petty, 1996, Metal-chelate affinity chromatography, in Current Protocols in Molecular Biology, volume 1-3 (1994-1998). Ed. by Ausubel, F. M., Brent, R., Kunston, R. E., Moore, D. D., Seidman, J. G., Smith, J. A. and Struhl, K. Published by John Wiley and sons, Inc., USA, Greene Publish. Assoc. & Wiley Interscience), glutathione S-transferase (GST; Smith, 1993, Methods Mol. Cell. Bio. 4:220-229), the E. Coli maltose binding protein (Guan et al., 1987, Gene 67:21-30), various cellulose binding domains (U.S. Pat. Nos. 5,496,934; 5,202,247; 5,137,819; Tomme et al., 1994, Protein Eng. 7:117-123), and the FLAG epitope (Short Protocols in Molecular Biology, 1999, Ed. Ausubel et al., John Wiley & Sons, Inc., Unit 10.11) etc. Other peptide tags are recognized by specific binding partners and thus facilitate isolation by affinity binding to the binding partner, which is preferably immobilized and/or on a solid support. As will be appreciated by those skilled in the art, many methods can be used to obtain the coding region of the above-mentioned peptide tags, including but not limited to, DNA cloning, DNA amplification, and synthetic methods. Some of the peptide tags and reagents for their detection and isolation are available commercially.

Samples from a subject can be obtained by any method known to the skilled artisan. In certain embodiments, the sample consists of nasal aspirate, throat swab, sputum or broncho-alveolar lavage.

(a) MINIREPLICON CONSTRUCTS

cDNA or minireplicon constructs that encode vRNAs containing a reporter gene can be used to rescue virus and also to identify the nucleotide sequences and proteins involved in amplification, expression, and incorporation of RNAs into virions. Any reporter gene known to the skilled artisan can be used with the invention. For example, reporter genes that can be used include, but are not limited to, genes that encode GFP, HRP, LUC, and AP. In one specific embodiment, the reporter gene that is used encodes CAT. In another specific embodiment of the invention, the reporter gene is flanked by leader and trailer sequences. The leader and trailer sequences that can be used to flank the reporter genes are those of any negative-sense virus, including, but not limited to, MPV, RSV, and APV. For example, the reporter gene can be flanked by the negative-sense hMPV or APV leader linked to the hepatitis delta ribozyme (Hep-d Ribo) and T7 polymerase termination (T-T7) signals, and the hMPV or APV trailer sequence preceded by the T7 RNA polymerase promoter.

In certain embodiments, the plasmid encoding the minireplicon is transfected into a host cell. In a more specific embodiment of the invention, hMPV is rescued in a host cell expressing T7 RNA polymerase, the N gene, the P gene, the L gene, and the M2.l gene. In certain embodiments, the host cell is transfected with plasmids encoding T7 RNA polymerase, the N gene, the P gene, the L gene, and the M2.1 gene. In other embodiments, the plasmid encoding the minireplicon is transfected into a host cell and the host cell is infected with a helper virus.

The hMPV minireplicon can be rescued using a number of polymerases, including, but not limited to, interspecies and intraspecies polymerases. In a certain embodiment, the hMPV minireplicon is rescued in a host cell expressing the minimal replication unit necessary for hMPV replication. For example, hMPV can be rescued from a cDNA using a number of polymerases, including, but not limited to, the polymerase of RSV, APV, MPV, or PIV. In a specific embodiment of the invention, hMPV is rescued using the polymerase of an RNA virus. In a more specific embodiment of the invention, hMPV is rescued using the polymerase of a negative stranded RNA virus. In an even more specific embodiment of the invention, hMPV is rescued using RSV polymerase. In another embodiment of the invention, hMPV is rescued using APV polymerase. In yet another embodiment of the invention, hMPV is rescued using an MPV polymerase. In another embodiment of the invention, hMPV is rescued using PIV polymerase.

In another embodiment of the invention, hMPV is rescued from a cDNA using a complex of hMPV polymerase proteins. For example, the hMPV minireplicon can be rescued using a polymerase complex consisting of the L, P, N, and M2.1 proteins. In another embodiment of the invention, the polymerase complex consists of the L, P, and N proteins. In yet another embodiment of the invention, the hMPV minireplicon can be rescued using a polymerase complex consisting of polymerase proteins from other viruses, such as, but not limited to, RSV, PIV, and APV. In particular, the hMPV minireplicon can be rescued using a polymerase complex consisting of the L, P, N, and M2.1 proteins of RSV, PIV, or APV. In yet another embodiment of the invention, the polymerase complex used to rescue the hMPV minireplicon consists of the L, P, and N proteins of RSV, PIV, or APV. In even another embodiment of the invention, different polymerase proteins from various viruses can be used to form the polymerase complex. In such an embodiment, the polymerase used to rescue the hMPV minireplicon can be formed by different components of the RSV, PIV, or APV polymerases. By way of example, and not meant to limit the possible combination, in forming a complex, the N protein can be encoded by the N gene of RSV, APV, or PIV, while the L protein is encoded by the L gene of RSV, APV, or PIV, and P protein can be encoded by the P gene of RSV, APV, or PIV. One skilled in the art would be able to determine the possible combinations that may be used to form the polymerase complex necessary to rescue the hMPV minireplicon. In the minireplicon system, the expression of a reporter gene is measured in order to confirm the successful rescue of the virus and also to characterize the virus. The expression level of the reporter gene and/or its activity can be assayed by any method known to the skilled artisan, such as, but not limited to, the methods described in section 5.8.2.

In certain, more specific, embodiments, the minireplicon comprises the following elements, in the order listed: T7 RNA Polymerase or RNA polymerase I, leader sequence, gene start, GFP, trailer sequence, Hepatitis delta ribozyme sequence or RNA polymerase I termination sequence. If T7 is used as RNA polymerase, Hepatitis delta ribozyme sequence should be used as termination sequence. If RNA polymerase I is used, RNA polymerase I termination sequence may be used as a termination signal. Dependent on the rescue system, the sequence of the minireplicon can be in the sense or antisense orientation. In certain embodiments, the leader sequence can be modified relative to the wild type leader sequence of hMPV. The leader sequence can optionally be preceded by an AC. The T7 promoter sequence can be with or without a G-doublet or triplet, where the G-doublet or triplet provides for increased transcription.

(b) Reporter Genes

In certain embodiments, assays for measurement of reporter gene expression in tissue culture or in animal models can be used with the methods of the invention. The nucleotide sequence of the reporter gene is cloned into the virus, such as APV, hMPV, hMPV/APV or APV/hMPV, wherein (i) the position of the reporter gene is changed and (ii) the length of the intergenic regions flanking the reporter gene are varied. Different combinations are tested to determine the optimal rate of expression of the reporter gene and the optimal replication rate of the virus comprising the reporter gene. The reporter gene can also be inserted into a minireplicon, see above.

The amount of reporter gene expression is representative of the activity of the minireplicon system or the virulence of the virus. The biochemical activity of the reporter gene product represents the expression level of the reporter gene. The total level of reporter gene activity depends also on the replication rate of the recombinant virus of the invention. Thus, to determine the true expression level of the reporter gene from the recombinant virus, the total expression level should be divided by the titer of the recombinant virus in the cell culture or the animal model.

Reporter genes that can be used with the methods of invention include, but are not limited to: CAT (chloramphenicol acetyltransferase—transfers radioactive acetyl groups to chloramphenicol or detection by thin layer chromatography and autoradiography); GAL (b-galactosidase—hydrolyzes colorless galactosides to yield colored products); GUS (b-glucuronidase—hydrolyzes colorless glucuronides to yield colored products); LUC (luciferase—oxidizes luciferin, emitting photons); GFP (green fluorescent protein—fluorescent protein without substrate); SEAP (secreted alkaline phosphatase—luminescence reaction with suitable substrates or with substrates that generate chromophores); HRP (horseradish peroxidase—in the presence of hydrogen oxide, oxidation of 3,3′,5,5′-tetramethylbenzidine to form a colored complex); and AP (alkaline phosphatase—luminescence reaction with suitable substrates or with substrates that generate chromophores).

The abundance of the reporter gene can be measured by, inter alia, Western blot analysis or Northern blot analysis or any other technique used for the quantification of transcription of a nucleotide sequence, the abundance of its mRNA its protein (see Short Protocols in Molecular Biology, Ausubel et al., (editors), John Wiley & Sons, Inc., 4^{th} edition, 1999). In certain embodiments, the activity of the reporter gene product is measured as a readout of reporter gene expression from the recombinant virus. For the quantification of the activity of the reporter gene product, biochemical characteristics of the reporter gene product can be employed. The methods for measuring the biochemical activity of the reporter gene products are well-known to the skilled artisan. A more detailed description of illustrative reporter genes is set forth below.

(c) Measurement of Incidence of Infection Rate

The incidence of infection can be determined by any method well-known in the art, for example, but not limited to, clinical samples (e.g., nasal swabs) can be tested for the presence of a virus of the invention by immunofluorescence assay (IFA) using an anti-APV-antigen antibody, an anti-hMPV-antigen antibody, an anti-APV-antigen antibody, and/or an antibody that is specific to the gene product of the heterologous nucleotide sequence, respectively.

In certain embodiments, samples containing intact cells can be directly processed, whereas isolates without intact cells should first be cultured on a permissive cell line (e.g. HEp-2 cells). In an illustrative embodiments, cultured cell suspensions should be cleared by centrifugation at, e.g., 300×g for 5 minutes at room temperature, followed by a PBS, pH 7.4 (Ca++ and Mg++free) wash under the same conditions. Cell pellets are resuspended in a small volume of PBS for analysis. Primary clinical isolates containing intact cells are mixed with PBS and centrifuged at 300×g for 5 minutes at room temperature. Mucus is removed from the interface with a sterile pipette tip and cell pellets are washed once more with PBS under the same conditions. Pellets are then resuspended in a small volume of PBS for analysis. Five to ten microliters of each cell suspension are spotted per 5 mm well on acetone washed 12-well HTC supercured glass slides and allowed to air dry. Slides are fixed in cold (−20° C.) acetone for 10 minutes. Reactions are blocked by adding PBS-1% BSA to each well followed by a 10 minute incubation at room temperature. Slides are washed three times in PBS-0.1% Tween-20 and air dried. Ten microliters of each primary antibody reagent diluted to 250 ng/ml in blocking buffer is spotted per well and reactions are incubated in a humidified 37° C. environment for 30 minutes. Slides are then washed extensively in three changes of PBS-0.1% Tween-20 and air dried. Ten microliters of appropriate secondary conjugated antibody reagent diluted to 250 ng/ml in blocking buffer are spotted per respective well and reactions are incubated in a humidified 37° C. environment for an additional 30 minutes. Slides are then washed in three changes of PBS-0.1% Tween-20. Five microliters of PBS-50% glycerol-10 mM Tris pH 8.0-1 mM EDTA are spotted per reaction well, and slides are mounted with cover slips. Each reaction well is subsequently analyzed by fluorescence microscopy at 200× power using a B-2A filter (EX 450-490 nm). Positive reactions are scored against an autofluorescent background obtained from unstained cells or cells stained with secondary reagent alone. Positive reactions are characterized by bright fluorescence punctuated with small inclusions in the cytoplasm of infected cells.

(d) Measurement of Serum Titer

Antibody serum titer can be determined by any method well-known in the art, for example, but not limited to, the amount of antibody or antibody fragment in serum samples can be quantitated by a sandwich ELISA. Briefly, the ELISA consists of coating microtiter plates overnight at 4° C. with an antibody that recognizes the antibody or antibody fragment in the serum. The plates are then blocked for approximately 30 minutes at room temperature with PBS-Tween-0.5% BSA. Standard curves are constructed using purified antibody or antibody fragment diluted in PBS-TWEEN-BSA, and samples are diluted in PBS-BSA. The samples and standards are added to duplicate wells of the assay plate and are incubated for approximately 1 hour at room temperature. Next, the non-bound antibody is washed away with PBS-TWEEN and the bound antibody is treated with a labeled secondary antibody (e.g., horseradish peroxidase conjugated goat-anti-human IgG) for approximately 1 hour at room temperature. Binding of the labeled antibody is detected by adding a chromogenic substrate specific for the label and measuring the rate of substrate turnover, e.g., by a spectrophotometer. The concentration of antibody or antibody fragment levels in the serum is determined by comparison of the rate of substrate turnover for the samples to the rate of substrate turnover for the standard curve at a certain dilution.

(e) Serological Tests

In certain embodiments of the invention, the presence of antibodies that bind to a component of a mammalian MPV is detected. In particular the presence of antibodies directed to a protein of a mammalian MPV can be detected in a subject to diagnose the presence of a mammalian MPV in the subject. Any method known to the skilled artisan can be used to detect the presence of antibodies directed to a component of a mammalian MPV.

In another embodiment, serological tests can be conducted by contacting a sample, from a host suspected of being infected with MPV, with an antibody to an MPV or a component thereof, and detecting the formation of a complex. In such an embodiment, the serological test can detect the presence of a host antibody response to MPV exposure. The antibody that can be used in the assay of the invention to detect host antibodies or MPV components can be produced using any method known in the art. Such antibodies can be engineered to detect a variety of epitopes, including, but not limited to, nucleic acids, amino acids, sugars, polynucleotides, proteins, carbohydrates, or combinations thereof. In another embodiment of the invention, serological tests can be conducted by contacting a sample from a host suspected of being infected with MPV, with an a component of MPV, and detecting the formation of a complex. Examples of such methods are well known in the art, including but are not limited to, direct immunofluoresence, ELISA, western blot, immunochromatography.

In an illustrative embodiment, components of mammalian MPV are linked to a solid support. In a specific embodiment, the component of the mammalian MPV can be, but is not limited to, the F protein or the G protein. Subsequently, the material that is to be tested for the presence of antibodies directed to mammalian MPV is incubated with the solid support under conditions conducive to the binding of the antibodies to the mammalian MPV components. Subsequently, the solid support is washed under conditions that remove any unspecifically bound antibodies. Following the washing step, the presence of bound antibodies can be detected using any technique known to the skilled artisan. In a specific embodiment, the mammalian MPV protein-antibody complex is incubated with detectably labeled antibody that recognizes antibodies that were generated by the species of the subject, e.g., if the subject is a cotton rat, the detectably labeled antibody is directed to rat antibodies, under conditions conducive to the binding of the detectably labeled antibody to the antibody that is bound to the component of mammalian MPV. In a specific embodiment, the detectably labeled antibody is conjugated to an enzymatic activity. In another embodiment, the detectably labeled antibody is radioactively labeled. The complex of mammalian MPV protein-antibody-detectably labeled antibody is then washed, and subsequently the presence of the detectably labeled antibody is quantified by any technique known to the skilled artisan, wherein the technique used is dependent on the type of label of the detectably labeled antibody.

(f) Microneutralization Assay

The ability of antibodies or antigen-binding fragments thereof to neutralize virus infectivity is determined by a microneutralization assay. This microneutralization assay is a modification of the procedures described by Anderson et al., (1985, J. Clin. Microbiol. 22:1050-1052, the disclosure of which is hereby incorporated by reference in its entirety). The procedure is also described in Johnson et al., 1999, J. Infectious Diseases 180:35-40, the disclosure of which is hereby incorporated by reference in its entirety.

Antibody dilutions are made in triplicate using a 96-well plate. 10⁶ TCID₅₀ of a mammalian MPV are incubated with serial dilutions of the antibody or antigen-binding fragments thereof to be tested for 2 hours at 37° C. in the wells of a 96-well plate. Cells susceptible to infection with a mammalian MPV, such as, but not limited to Vero cells (2.5×10⁴) are then added to each well and cultured for 5 days at 37° C. in 5% CO₂. After 5 days, the medium is aspirated and cells are washed and fixed to the plates with 80% methanol and 20% PBS. Virus replication is then determined by viral antigen, such as F protein expression. Fixed cells are incubated with a biotin-conjugated anti-viral antigen, such as anti-F protein monoclonal antibody (e.g., pan F protein, C-site-specific MAb 133-1H) washed and horseradish peroxidase conjugated avidin is added to the wells. The wells are washed again and turnover of substrate TMB (thionitrobenzoic acid) is measured at 450 nm. The neutralizing titer is expressed as the antibody concentration that causes at least 50% reduction in absorbency at 450 nm (the OD₄₅₀) from virus-only control cells.

The cells can be infected with the respective virus for four hours prior to addition of antibody and the read-out is in terms of presence of absence of fusion of cells (Taylor et al., 1992, J. Gen. Virol. 73:2217-2223).

(g) Phylogenetic Analysis

Many methods or approaches are available to analyze phylogenetic relationship; these include distance, maximum likelihood, and maximum parsimony methods (Swofford, D L., et. al., Phylogenetic Inference. In Molecular Systematics. Eds. Hillis, D M, Mortiz, C, and Mable, BK. 1996. Sinauer Associates: Mass., USA. pp. 407-514; Felsenstein, J., 1981, J. Mol. Evol. 17:368-376). In addition, bootstrapping techniques are an effective means of preparing and examining confidence intervals of resultant phylogenetic trees (Felsenstein, J., 1985, Evolution. 29:783-791). Any method or approach using nucleotide or peptide sequence information to compare mammalian MPV isolates can be used to establish phylogenetic relationships, including, but not limited to, distance, maximum likelihood, and maximum parsimony methods or approaches. Any method known in the art can be used to analyze the quality of phylogenetic data, including but not limited to bootstrapping. Alignment of nucleotide or peptide sequence data for use in phylogenetic approaches, include but are not limited to, manual alignment, computer pairwise alignment, and computer multiple alignment. One skilled in the art would be familiar with the preferable alignment method or phylogenetic approach to be used based upon the information required and the time allowed.

In one embodiment, a DNA maximum likehood method is used to infer relationships between hMPV isolates. In another embodiment, bootstrapping techniques are used to determine the certainty of phylogenetic data created using one of said phylogenetic approaches. In another embodiment, jumbling techniques are applied to the phylogenetic approach before the input of data in order to minimize the effect of sequence order entry on the phylogenetic analyses. In one specific embodiment, a DNA maximum likelihood method is used with bootstrapping. In another specific embodiment, a DNA maximum likelihood method is used with bootstrapping and jumbling. In another more specific embodiment, a DNA maximum likelihood method is used with 50 bootstraps. In another specific embodiment, a DNA maximum likelihood method is used with 50 bootstraps and 3 jumbles. In another specific embodiment, a DNA maximum likelihood method is used with 100 bootstraps and 3 jumbles.

In one embodiment, nucleic acid or peptide sequence information from an isolate of hMPV is compared or aligned with sequences of other hMPV isolates. The amino acid sequence can be the amino acid sequence of the L protein, the M protein, the N protein, the P protein, or the F protein. In another embodiment, nucleic acid or peptide sequence information from an hMPV isolate or a number of hMPV isolates is compared or aligned with sequences of other viruses. In another embodiment, phylogenetic approaches are applied to sequence alignment data so that phylogenetic relationships can be inferred and/or phylogenetic trees constructed. Any method or approach that uses nucleotide or peptide sequence information to compare hMPV isolates can be used to infer said phylogenetic relationships, including, but not limited to, distance, maximum likelihood, and maximum parsimony methods or approaches.

Other methods for the phylogenetic analysis are disclosed in International Patent Application PCT/NL02/00040, published as WO 02/057302, which is incorporated in its entirety herein. In particular, PCT/NL02/00040 discloses nucleic acid sequences that are suitable for phylogenetic analysis at page 12, line 27 to page 19, line 29, which is incorporated herein by reference.

For the phylogenetic analyses it is most useful to obtain the nucleic acid sequence of a non-MPV as outgroup with which the virus is to be compared, a very useful outgroup isolate can be obtained from avian pneumovirus serotype C (APV-C).

Many methods and programs are known in the art and can be used in the inference of phylogenetic relationships, including, but not limited to BioEdit, ClustalW, TreeView, and NJPlot. Methods that would be used to align sequences and to generate phylogenetic trees or relationships would require the input of sequence information to be compared. Many methods or formats are known in the art and can be used to input sequence information, including, but not limited to, FASTA, NBRF, EMBL/SWISS, GDE protein, GDE nucleotide, CLUSTAL, and GCG/MSF. Methods that would be used to align sequences and to generate phylogenetic trees or relationships would require the output of results. Many methods or formats can be used in the output of information or results, including, but not limited to, CLUSTAL, NBRF/PIR, MSF, PHYLIP, and GDE. In one embodiment, ClustalW is used in conjunction with DNA maximum likelihood methods with 100 bootstraps and 3 jumbles in order to generate phylogenetic relationships.

(h) Direct Immunofluoresence Assay (DIF) Method

Nasopharyngeal aspirateples from patients suffering from RTI can be analyzed by DIF as described (Rothbarth et. al., 1999, J. of Virol. Methods 78:163-169). Samples are stored at −70° C. In short, nasopharyngeal aspirates are diluted with 5 ml Dulbecco MEM (BioWhittaker, Walkersville, Md.) and thoroughly mixed on a vortex mixer for one minute. The suspension is centrifuged for ten minutes at 840×g. The sediment is spread on a multispot slide (Nutacon, Leimuiden, The Netherlands) and the supernatant is used for virus isolation. After drying, the cells are fixed in acetone for one minute at room temperature. After the slides are washed, they are incubated for 15 minutes at 37° C. with commercially available FITC-labeled anti-sera specific for viruses such as influenza A and B, hRSV and hPIV 1 to 3 (Dako, Glostrup, Denmark). After three washings in PBS and one in tap water, the slides are submerged in a glycerol/PBS solution (Citifluor, UKO, Canterbury, UK) and covered. The slides are then analyzed using a Axioscop fluorescence microscope.

(i) Virus Culture of MPV

The detection of the virus in a cultivated sample from a host is a direct indication of the host's current and/or past exposure or infection with the virus. Samples that display CPE after the first passage are used to inoculate sub-confluent mono-layers of tMK cells in media in 24 well plates. Cultures are checked for CPE daily and the media is changed once a week. Since CPE can differ for each isolate, all cultures are tested at day 12 to 14 with indirect IFA using ferret antibodies against the new virus isolate. Positive cultures are freeze-thawed three times, after which the supernatants are clarified by low-speed centrifugation, aliquoted and stored frozen at −70° C. The 50% tissue culture infectious doses (TCID₅₀) of virus in the culture supernatants are determined as described (Lennette, D. A. et al. In: DIAGNOSTIC PROCEDURES FOR VIRAL, RICKETTSIAL, AND CHLAMYDIAL INFECTIONS, 7th ed. (eds. Lennette, E. H., Lennette, D. A. & Lennette, E. T.) 3-25; 37-138; 431-463; 481-494; 539-563 (American Public Health Association, Washington, 1995)).

(j) Antigen Detection by Indirect Immunofluoresence Assays (IFA)

Antibodies can be used to visualize viral proteins in infected cells or tissues. Indirect immunofluorescence assay (IFA) is a sensitive approach in which a second antibody coupled to a fluorescence indicator recognizes a general epitope on the virus-specific antibody. IFA is more advantageous than DIF because of its higher level of sensitivity.

In order to perform the indirect IFA, collected specimens are diluted with 5 ml Dulbecco MEM medium (BioWhittaker, Walkersville, Md.) and thoroughly mixed on a vortex mixer for one minute. The suspension is then centrifuged for ten minutes at 840×g. The sediment is spread on a multispot slide. After drying, the cells are fixed in acetone for 1 minute at room temperature. Alternatively, virus is cultured on tMK cells in 24 well slides containing glass slides. These glass slides are washed with PBS and fixed in acetone for 1 minute at room temperature.

Two indirect IFAs can be performed. In the first indirect IFA, slides containing infected tMK cells are washed with PBS, and then incubated for 30 minutes at 37° C. with virus specific antisera. Monoclonal antibodies against influenza A, B and C, hPIV type 1 to 3, and hRSV can be used. For hPIV type 4, mumps virus, measles virus, sendai virus, simian virus type 5, and New-Castle Disease virus, polyclonal antibodies (RIVM) and ferret and guinea pig reference sera are used. After three washings with PBS and one wash with tap water, the slides are stained with secondary antibodies directed against the sera used in the first incubation. Secondary antibodies for the polyclonal antisera are goat-anti-ferret (KPL, Guilford, UK, 40 fold diluted), mouse-anti-rabbit (Dako, Glostrup, Denmark, 20 fold diluted), rabbit-anti-chicken (KPL, 20 fold dilution) and mouse-anti-guinea pig (Dako, 20 fold diluted).

In the second IFA, after washing with PBS, the slides are incubated for 30 minutes at 37° C. with 20 polyclonal antibodies at a dilution of 1:50 to 1:100 in PBS. Immunized ferrets and guinea pigs are used to obtain polyclonal antibodies, but these antibodies can be raised in various animals, and the working dilution of the polyclonal antibody can vary for each immunization. After three washes with PBS and one wash with tap water, the slides are incubated at 37° C. for 30 minutes with FITC labeled goat-anti-ferret antibodies (KPL, Guilford, UK, 40 fold diluted). After three washes in PBS and one in tap water, the slides are included in a glycerol/PBS solution (Citifluor, UKO, Canterbury, UK) and covered. The slides are analyzed using an Axioscop fluorescence microscope (Carl Zeiss B. V., Weesp, the Netherlands).

(k) Haemagglutination Assays, Chloroform Sensitivity Tests and Electron Microscopy

Different characteristics of a virus can be utilized for the detection of the virus. For example, many virus contain proteins that can bind to erythrocytes resulting in a lattice. This property is called hemagglutination and can be used in hemagglutination assays for detection of the virus. Virus may also be visualized under an electron microscope (EM) or detected by PCR techniques.

Hemagglutination assays and chloroform sensitivity tests can be performed as described (Osterhaus et al., 1985, Arch.of Virol 86:239-25; Rothbarth et al., J of Virol Methods 78:163-169).

For EM analyses, virus can be concentrated from infected cell culture supernatants in a micro-centrifuge at 4° C. at 17000×g, after which the pellet is resuspended in PBS and inspected by negative contrast EM.

(l) Detection of hMPV/AVP Antibodies of IgG, IgA and IgM Classes

Specific antibodies to viruses are formed during the course of infection/illness. Thus, detection of virus-specific antibodies in a host is an indicator of current and/or past infections of the host with that virus.

The indirect enzyme immunoassay (EIA) can be used to detect the IgG class of hMPV antibodies. This assay is performed in microtitre plates essentially as described previously (Rothbarth et al., 1999, J. of Vir. Methods 78:163-169). Briefly, concentrated hMPV is solubilized by treatment with 1% Triton X-100. After determination of the optimal working dilution by checkerboard titration, it is coated for 16 hr at room temperature into microtitre plates in PBS. Subsequently, 100 ul volumes of 1:100 diluted human serum samples in EIA buffer are added to the wells and incubated for 1 hour at 37° C. Binding of human IgG is detected by adding a goat anti-human IgG peroxidase conjugate (Biosource, USA), adding TMB as substrate developed plates and Optical Density (OD) is measured at 450 nm. The results are expressed as the S(ignal)/N(egative) ratio of the OD. A serum is considered positive for IgG if the S/N ratio was beyond the negative control plus three times the standard.

The hMPV antibodies of the IgM and IgA classes can be detected in sera by capture EIA essentially as described previously (Rothbarth et al., 1999, J Vir Methods 78:163-169). For the detection of IgA and IgM, commercially available microtiter plates coated with anti human IgM or IgA specific monoclonal antibodies can be used. Sera can be diluted 1:100. After incubation of 1 hour at 37° C., an optimal working dilution of hMPV is added to each well (100 μl) before incubation for 1 hour at 37° C. After washing, polyclonal anti-hMPV antibody labeled with peroxidase is added, and the plate is incubated 1 hour at 37° C. Adding TMB as a substrate the plates are developed, and OD is measured at 450 rim. The results are expressed as the S(ignal)/N(egative) ratio of the OD. A positive result is indicated for IgG when the S/N ratio is beyond the negative control plus three times the standard.

AVP antibodies are detected in an AVP inhibition assay. The protocol for the APV inhibition test is included in the APV-Ab SVANOVIR® enzyme immunoassay that is manufactured by SVANOVA Biotech AB, Uppsala Science Park Glunten SE-751 83 Uppsala Sweden. The results are expressed as the S(ignal)/N(egative ratio of the OD. A serum is considered positive for IgG, if the S/N ratio was beyond the negative control plus three times the standard.

(m) Detection of Antibodies in Humans, Mammals, Ruminants or Other Animals by Indirect IFA

For the detection of virus specific antibodies, infected tMK cells with MPV can be fixed with acetone on coverslips (as described above), washed with PBS and incubated 30 minutes at 37° C. with serum samples at a 1 to 16 dilution. After two washes with PBS and one with tap water, the slides are incubated for 30 minutes at 37° C. with FITC-labeled secondary antibodies to the species used (Dako). Slides are processed as described above. Antibodies can be labeled directly with a fluorescent dye, which will result in a direct immunofluorescence assay. FITC can be replaced with any fluorescent dye.

(n) Virus Neutralization Assay

When a subject is infected with a virus, an array of antibodies against the virus are produced. Some of these antibodies can bind virus particles and neutralize their infectivity. Virus neutralization assays (VN) are usually conducted by mixing dilutions of serum or monoclonal antibody with virus, incubating them, and assaying for remaining infectivity with cultured cells, embryonated eggs, or animals. Neutralizing antibodies can be used to define type-specific antigens on the virus particle, e.g., neutralizing antibodies could be used to define serotypes of a virus. Additionally, broadly neutralizing antibodies may also exist.

VN assays can be performed with serial two-fold dilutions of human and animal sera starting at an eight-fold dilution. Diluted sera are incubated for one hour with 100 TCID₅₀ of virus before inoculation of tMK cells grown in 96 well plates, after which the plates can be centrifuged at 840×g. The media is changed after three and six days and IFA was conducted with FTIC-labeled ferret antibodies against MPV 8 days after inoculation. The VN titre can be defined as the lowest dilution of the serum sample resulting in negative IFA and inhibition of CPE in cell cultures.

(O)RNA Isolation

The presence of viruses in a host can also be diagnosed by detecting the viral nucleic acids in samples taken from the host. RNA can be isolated from the supernatants of infected cell cultures or sucrose gradient fractions using a High Pure RNA Isolation kit, according to instructions from the manufacturer (Roche Diagnostics, Ahnere, The Netherlands). RNA can also be isolated following other procedures known in the art (see, e.g., CURRENT PROTOCOLS IN MOLECULAR BIOLOGY, volume 1-3 (1994-1998). Ed. by Ausubel, F. M. et al., Published by John Wiley and sons, Inc., USA).

(p) RT-PCR to Detect/Diagnose MPV

Detection of the virus in a biological sample can be done using methods that copy or amplify the genomic material of the virus. A one-step RT-PCR can be performed in 50 μl reactions containing 50 mM Tris.HCl pH 8.5, 50 mM NaCl, 4 mM MgCl₂, 2 mM dithiotreitol, 200 μM each dNTP, 10 units recombinant RNAsin (Promega, Leiden, the Netherlands), 10 units AMV RT (Promega, Leiden, The Netherlands), 5 units Amplitaq Gold DNA polymerase (PE Biosystems, Nieuwerkerk aan de Ijssel, The Netherlands) and 5 μl RNA. Cycling conditions can be 45 min. at 42° C. and 7 min. at 95° C. once, 1 min at 95° C., 2 min. at 42° C. and 3 min. at 72° C. repeated 40 times and 10 min. at 72° C. once.

(q) RAP PCR

RAP-PCR can be performed essentially as described (Welsh et al., 1992, NAR 20:4965-4970). Essentially, the RAP PCR can be performed as follows: For the RT reaction, 2 μl of RNA are used in a 10 μl reaction containing 10 ng/μl oligonucleotide, 10 mM dithiotreitol, 500 μm each dNTP, 25 mM Tris-HCl pH 8.3, 75 mM KCl and 3 mM MgCl₂. The reaction mixture is incubated for 5 minutes at 70° C. and 5 minutes at 37° C., after which 200 units Superscript RT enzyme (LifeTechnologies) are added. The incubation at 37° C. is continued for 55 minutes and the reaction is terminated by a 5 minute incubation at 72° C. The RT mixture is diluted to give a 50 μl PCR reaction containing 8 ng/μl oligonucleotide, 3001 each dNTP, 15 mM Tris-HCl pH 8.3, 65 mM KCl, 3.0 mM MgCL₂ and 5 units Taq DNA polymerase (FE Biosystems). Cycling conditions are 5 minutes at 94° C., 5 minutes at 40° C., and 1 minute at 72° C. once, followed by 1 minute at 94° C., 2 minutes at 56° C. and 1 minute at 72° C. repeated 40 times, and 5 minutes at 72° C. once.

(r) Capture Anti-MPV IgM EIA Using a Recombinant Nucleoprotein.

In order to detect the hMPV virus, an immunological assay that detects the presence of the antibodies in a variety of hosts. In one example, antibodies to the N protein are used because it is the most abundant protein that is produced. This feature is due the transciptional gradient that occurs across the genome of the virus.

A capture IgM EIA using the recombinant nucleoprotein or any other recombinant protein as antigen can be performed by modification of assays as previously described by Erdman et al., 1990, J. Clin.Microb. 29: 1466-1471.

Affinity purified anti-human IgM capture antibody (or against other species), such as that obtained from Dako, is added to wells of a microtiter plate in a concentration of 250 ng per well in 0.1 M carbonate buffer pH 9.6. After overnight incubation at room temperature, the plates are washed two times with PBS/0.05% Tween. 100 μl of test serum diluted 1:200 to 1:1000 in ELISA buffer is added to triplicate wells and incubated for 1 hour at 37° C. The plates are then washed two times with in PBS/0.05% Tween.

The freeze-thawed (infected with recombinant virus) Sf21 cell lysate is diluted 1:100 to 1:500 in ELISA buffer is added to the wells and incubated for 2 hours at 37° C. Uninfected cell lysate serves as a negative control and is run in duplicate wells. The plates are then washed three times in PBS/0.05% Tween and incubated for 1 hour at 37° C. with 100 μl of a polyclonal antibody against MPV in a optimal dilution in ELISA buffer. After 2 washes with PBS/0.05% Tween, the plates are incubated with horseradish peroxide labeled secondary antibody (such as rabbit anti ferret), and the plates are incubated 20 minutes at 37° C.

The plates are then washed five times in PBS/0/05% Tween, incubated for 15 minutes at room temperature with the enzyme substrate TMB, 3,3,5,5 tetramethylbenzidine, as, for instance obtained from “Sigma”, and the reaction is stopped with 100 μl of 2M phosphoric acid. Colormetric readings are measured at 450 nm using automated microtiter plate reader.

The sensitivities of the capture IgM EIAs using the recombinant nucleoprotein (or other recombinant protein) and whole MPV virus are compared using acute—and convalescent—phase serum pairs form persons with clinical MPV virus infection. The specificity of the recombinant nucleoprotein capture EIA is determined by testing serum specimens from healthy persons and persons with other paramyxovirus infections.

Potential for EIAs for using recombinant MPV fusion and glycoprotein proteins produced by the baculovirus expression.

The glycoproteins G and F are the two transmembraneous envelope glycoproteins of the MPV virion and represent the major neutralisation and protective antigens. The expression of these glycoproteuns in a vector virus system sych as a baculovinus system provides a source of recombinant antigens for use in assays for detection of MPV specific antibodies. Moreover, their use in combination with the nucleoprotein, for instance, further enhances the sensitivity of enzyme immunoassays in the detection of antibodies against MPV.

A variety of other immunological assays (Current Protocols in Immunology, volume 1-3. Ed. by Coligan, J. E., Kruisbeek, A. M., Margulies, D. H., Shevach, E. M. and Strobe, W. Published by John Wiley and sons, Inc., USA) may be used as alternative methods to those described here.

In order to find virus isolates nasopharyngeal aspirates, throat and nasal swabs, broncheo alveolar lavages and throat swabs preferable from but not limited to humans, carnivores (dogs, cats, seals etc.), horses, ruminants (cattle, sheep, goats etc.), pigs, rabbits, birds (poultry, ostridges, etc) can be examined. From birds, cloaca and intestinal swabs and droppings can be examined as well. For all samples, serology (antibody and antigen detection etc.), virus isolation and nucleic acid detection techniques can be performed for the detection of virus. Monoclonal antibodies can be generated by immunizing mice (or other animals) with purified MPV or parts thereof (proteins, peptides) and subsequently using established hybridoma technology (Current Protocols in Immunology, Published by John Wiley and sons, Inc., USA). Alternatively, phage display technology can be used for this purpose (Current Protocols in Immunology, Published by John Wiley and sons, Inc., USA). Similarly, polyclonal antibodies can be obtained from infected humans or animals, or from immunised humans or animals (Current Protocols in Immunology, Published by John Wiley and sons, Inc., USA).

The detection of the presence or absence of NS1 and NS2 proteins can be performed using western-blotting, IFA, immuno precipitation techniques using a variety of antibody preparations. The detection of the presence or absence of NS1 and NS2 genes or homologues thereof in virus isolates can be performed using PCR with primer sets designed on the basis of known NS1 and/or NS2 genes as well as with a variety of nucleic acid hybridisation techniques.

### 5.9 Formulations of Vaccines, Antibodies and Antivirals

A pharmaceutical composition comprising a virus, a nucleic acid, a proteinaceous molecule or fragment thereof, an antigen and/or an antibody according to the invention can for example be used in a method for the treatment or prevention of a MPV infection and/or a respiratory illness comprising providing an individual with a pharmaceutical composition according to the invention. This is most useful when said individual comprises a human, specifically when said human is below 5 years of age, since such infants and young children are most likely to be infected by a human MPV as provided herein. Generally, in the acute phase patients will suffer from upper respiratory symptoms predisposing for other respiratory and other diseases. Also lower respiratory illnesses may occur, predisposing for more and other serious conditions. The compositions of the invention can be used for the treatment of immuno-compromised individuals including cancer patients, transplant recipients and the elderly.

In certain embodiments of the invention, the vaccine of the invention comprises mutant mMPV, or, more specifically, a mutant hMPV. In a preferred embodiment, the mammalian metapneumovirus to be used in a vaccine formulation has an attenuated phenotype.

The invention provides vaccine formulations for the prevention and treatment of infections with PIV, RSV, APV, and/or hMPV. In certain embodiments, the vaccine of the invention comprises recombinant and chimeric viruses of the invention. In a specific embodiment, the vaccine comprises APV and the vaccine is used for the prevention and treatment for hMPV infections in humans. Without being bound by theory, because of the high degree of homology of the F protein of APV with the F protein of hMPV, infection with APV will result in the production of antibodies in the host that will cross-react with hMPV and protect the host from infection with hMPV and related diseases.

In another specific embodiment, the vaccine comprises hMPV and the vaccine is used for the prevention and treatment for APV infection in birds, such as, but not limited to, in turkeys. Without being bound by theory, because of the high degree of homology of the F protein of APV with the F protein of hMPV, infection with hMPV will result in the production of antibodies in the host that will cross-react with APV and protect the host from infection with APV and related diseases.

In a specific embodiment, the invention encompasses the use of recombinant and chimeric APV/hMPV viruses which have been modified in vaccine formulations to confer protection against APV and/or hMPV. In certain embodiments, APV/hMPV is used in a vaccine to be administered to birds, to protect the birds from infection with APV. Without being bound by theory, the replacement of the APV gene or nucleotide sequence with a hMPV gene or nucleotide sequence results in an attenuated phenotype that allows the use of the chimeric virus as a vaccine. In other embodiments the APV/hMPV chimeric virus is administered to humans. Without being bound by theory the APV viral vector provides the attenuated phenotype in humans and the expression of the hMPV sequence elicits a hMPV specific immune response.

In a specific embodiment, the invention encompasses the use of recombinant and chimeric hMPV/APV viruses which have been modified in vaccine formulations to confer protection against APV and/or hMPV. In certain embodiments, hMPV/APV is used in a vaccine to be administered to humans, to protect the human from infection with hMPV. Without being bound by theory, the replacement of the hMPV gene or nucleotide sequence with a APV gene or nucleotide sequence results in an attenuated phenotype that allows the use of the chimeric virus as a vaccine. In other embodiments the hMPV/APV chimeric virus is administered to birds. Without being bound by theory the hMPV backbone provides the attenuated phenotype in birds and the expression of the APV sequence elicits an APV specific immune response.

Due to the high degree of homology among the F proteins of different viral species, the vaccine formulations of the invention can be used for protection from viruses different from the one from which the heterologous nucleotide sequence encoding the F protein was derived. In a specific exemplary embodiment, a vaccine formulation contains a virus comprising a heterologous nucleotide sequence derived from an avian pneumovirus type A, and the vaccine formulation is used to protect from infection by avian pneumovirus type A and avian pneumovirus type B. The invention encompasses vaccine formulations to be administered to humans and animals which are useful to protect against APV, including APV-C and APV-D, hMPV, PIV, influenza, RSV, Sendai virus, mumps, laryngotracheitis virus, simianvirus 5, human papillomavirus, measles, mumps, as well as other viruses and pathogens and related diseases. The invention further encompasses vaccine formulations to be administered to humans and animals which are useful to protect against human metapneumovirus infections and avian pneumovirus infections and related diseases.

In one embodiment, the invention encompasses vaccine formulations which are useful against domestic animal disease causing agents including rabies virus, feline leukemia virus (FLV) and canine distemper virus. In yet another embodiment, the invention encompasses vaccine formulations which are useful to protect livestock against vesicular stomatitis virus, rabies virus, rinderpest virus, swinepox virus, and further, to protect wild animals against rabies virus.

In a specific embodiment, the recombinant virus is non-pathogenic to the subject to which it is administered. In this regard, the use of genetically engineered viruses for vaccine purposes may desire the presence of attenuation characteristics in these strains. The introduction of appropriate mutations (e.g., deletions) into the templates used for transfection may provide the novel viruses with attenuation characteristics. For example, specific missense mutations which are associated with temperature sensitivity or cold adaption can be made into deletion mutations. These mutations should be more stable than the point mutations associated with cold or temperature sensitive mutants and reversion frequencies should be extremely low.

The mutant mMPV of the invention may further have “suicide” characteristics. Such viruses would go through only one or a few rounds of replication within the host. When used as a vaccine, the recombinant virus would go through limited replication cycle(s) and induce a sufficient level of immune response but it would not go further in the human host and cause disease. Recombinant viruses lacking one or more of the genes of wild type APV and hMPV, respectively, or possessing mutated genes as compared to the wild type strains would not be able to undergo successive rounds of replication. Defective viruses can be produced in cell lines which permanently express such a gene(s). Viruses lacking an essential gene(s) will be replicated in these cell lines but when administered to the human host will not be able to complete a round of replication. Such preparations may transcribe and translate—in this abortive cycle—a sufficient number of genes to induce an immune response. Alternatively, larger quantities of the strains could be administered, so that these preparations serve as inactivated (killed) virus vaccines. For inactivated vaccines, it is preferred that the heterologous gene product be expressed as a viral component, so that the gene product is associated with the virion. The advantage of such preparations is that they contain native proteins and do not undergo inactivation by treatment with formalin or other agents used in the manufacturing of killed virus vaccines. Alternatively, recombinant virus of the invention made from cDNA may be highly attenuated so that it replicates for only a few rounds.

A mutant mMPV of the invention can be effective as a vaccine even if the attenuated virus is incapable of causing a cell to generate new infectious viral particles because the viral proteins are inserted in the cytoplasmic membrane of the host thus stimulating an immune response.

In another embodiment of this aspect of the invention, inactivated vaccine formulations may be prepared using conventional techniques to “kill” the chimeric viruses. Inactivated vaccines are “dead” in the sense that their infectivity has been destroyed. Ideally, the infectivity of the virus is destroyed without affecting its immunogenicity. In order to prepare inactivated vaccines, the chimeric virus may be grown in cell culture or in the allantois of the chick embryo, purified by zonal ultracentrifugation, inactivated by formaldehyde or β-propiolactone, and pooled. The resulting vaccine is usually inoculated intramuscularly.

Inactivated viruses may be formulated with a suitable adjuvant in order to enhance the immunological response. Such adjuvants may include but are not limited to mineral gels, e.g., aluminum hydroxide; surface active substances such as lysolecithin, pluronic polyols, polyanions; peptides; oil emulsions; and potentially useful human adjuvants such as BCG, Corynebacterium parvum, ISCOMS and virosomes.

Many methods may be used to introduce the vaccine formulations described above, these include but are not limited to oral, intradermal, intramuscular, intraperitoneal, intravenous, subcutaneous, percutaneous, and intranasal and inhalation routes. It may be preferable to introduce the chimeric virus vaccine formulation via the natural route of infection of the pathogen for which the vaccine is designed.

In certain embodiments, the invention relates to immunogenic compositions. The immunogenic compositions comprise a mammalian MPV. In a specific embodiment, the immunogenic composition comprises a human MPV. In certain embodiments, the immunogenic composition comprises an attenuated mammalian MPV or an attenuated human MPV. In certain embodiments, the immunogenic composition further comprises a pharmaceutically acceptable carrier.

In certain embodiments, administration of a mutant mMPV is combined with heptad repeats of the F protein of the mMPV. For the use of heptad repeats for the inhibition of virus-cell fusion, see section 5.16 of International Patent Application No. PCT/U504/12724 (published as WO 04/096993).

### 5.10 Dosage Regimens, Administration and Formulations

The present invention provides vaccines and immunogenic preparations comprising the mutant mMPV of the invention. Particularly, the vaccines or immunogenic formulations of the invention provide protection against or reduce the symptoms of a respiratory tract infections in a host.

A recombinant virus and/or a vaccine or immunogenic formulation of the invention can be administered alone or in combination with other vaccines. Preferably, a vaccine or immunogenic formulation of the invention is administered in combination with other vaccines or immunogenic formulations that provide protection against respiratory tract diseases, such as but not limited to, respiratory syncytial virus vaccines, influenza vaccines, measles vaccines, mumps vaccines, rubella vaccines, pneumococcal vaccines, rickettsia vaccines, staphylococcus vaccines, whooping cough vaccines or vaccines against respiratory tract cancers. In a preferred embodiment, the virus and/or vaccine of the invention is administered concurrently with pediatric vaccines recommended at the corresponding ages. For example, at two, four or six months of age, the virus and/or vaccine of the invention can be administered concurrently with DtaP (IM), Hib (IM), Polio (IPV or OPV) and Hepatitis B (IM). At twelve or fifteen months of age, the virus and/or vaccine of the invention can be administered concurrently with Hib (IM), Polio (IPV or OPV), MMRII® (SubQ); Varivax® (SubQ), and hepatitis B (IM). The vaccines that can be used with the methods of invention are reviewed in various publications, e.g., The Jordan Report 2000, Division of Microbiology and Infectious Diseases, National Institute of Allergy and Infectious Diseases, National Institutes of Health, United States, the content of which is incorporated herein by reference in its entirety.

A vaccine or immunogenic formulation of the invention may be administered to a subject per se or in the form of a pharmaceutical or therapeutic composition. Pharmaceutical compositions comprising an adjuvant and an immunogenic antigen of the invention (e.g., a virus, a chimeric virus, a mutated virus) may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the immunogenic antigen of the invention into preparations which can be used pharmaceutically. Proper formulation is, amongst others, dependent upon the route of administration chosen.

When a vaccine or immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum hydroxide, aluminum phosphate gel, Freund's Complete Adjuvant, Freund's Incomplete Adjuvant, squalene or squalane oil-in-water adjuvant formulations, biodegradable and biocompatible polyesters, polymerized liposomes, triterpenoid glycosides or saponins (e.g., QuilA and QS-21, also sold under the trademark STIMULON, ISCOPREP), N-acetyl-muramyl-L-threonyl-D-isoglutamine (Threonyl-MDP, sold under the trademark TERMURTIDE), LPS, monophosphoryl Lipid A (3D-MLAsold under the trademark MPL).

The subject to which the vaccine or an immunogenic composition of the invention is administered is preferably a mammal, most preferably a human, but can also be a non-human animal, including but not limited to, primates, cows, horses, sheep, pigs, fowl (e.g., chickens, turkeys), goats, cats, dogs, hamsters, mice and rodents.

Many methods may be used to introduce the vaccine or the immunogenic composition of the invention, including but not limited to, oral, intradermal, intramuscular, intraperitoneal, intravenous, subcutaneous, percutaneous, intranasal and inhalation routes, and via scarification (scratching through the top layers of skin, e.g., using a bifurcated needle).

For topical administration, the vaccine or immunogenic preparations of the invention may be formulated as solutions, gels, ointments, creams, suspensions, etc. as are well-known in the art. For administration intranasally or by inhalation, the preparation for use according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents. Alternatively, the proteins may be in powder form for constitution with a suitable vehicle, e.g., sterile pyrogen-free water, before use.

Determination of an effective amount of the vaccine or immunogenic formulation for administration is well within the capabilities of those skilled in the art, especially in light of the detailed disclosure provided herein.

An effective dose can be estimated initially from in vitro assays. For example, a dose can be formulated in animal models to achieve an induction of an immunity response using techniques that are well known in the art. One having ordinary skill in the art could readily optimize administration to all animal species based on results described herein. Dosage amount and interval may be adjusted individually. For example, when used as an immunogenic composition, a suitable dose is an amount of the composition that when administered as described above, is capable of eliciting an antibody response. When used as a vaccine, the vaccine or immunogenic formulations of the invention may be administered in about 1 to 3 doses for a 1-36 week period. Preferably, 1 or 2 doses are administered, at intervals of about 2 weeks to about 4 months, and booster vaccinations may be given periodically thereafter. Alternate protocols may be appropriate for individual animals. A suitable dose is an amount of the vaccine formulation that, when administered as described above, is capable of raising an immunity response in an immunized animal sufficient to protect the animal from an infection for at least 4 to 12 months. In general, the amount of the antigen present in a dose ranges from about 1 pg to about 100 mg per kg of host, typically from about 10 pg to about 1 mg, and preferably from about 100 pg to about 1 μg. Suitable dose range will vary with the route of injection and the size of the patient, but will typically range from about 0.1 mL to about 5 mL.

In a specific embodiment, the viruses and/or vaccines of the invention are administered at a starting single dose of at least 10³ TCID₅₀, at least 10⁴ TCID₅₀, at least 10⁵ TCID₅₀, at least 10⁶ TCID₅₀. In another specific embodiment, the virus and/or vaccines of the invention are administered at multiple doses. In a preferred embodiment, a primary dosing regimen at 2, 4, and 6 months of age and a booster dose at the beginning of the second year of life are used. More preferably, each dose of at least 10⁵ TCID₅₀, or at least 10⁶ TCID₅₀ is given in a multiple dosing regimen.

(a) Challenge Studies

This assay is used to determine the ability of the recombinant viruses of the invention and of the vaccines of the invention to prevent lower respiratory tract viral infection in an animal model system, such as, but not limited to, cotton rats or hamsters. The recombinant virus and/or the vaccine can be administered by intravenous (IV) route, by intramuscular (IM) route or by intranasal route (IN). The recombinant virus and/or the vaccine can be administered by any technique well-known to the skilled artisan. This assay is also used to correlate the serum concentration of antibodies with a reduction in lung titer of the virus to which the antibodies bind.

On day 0, groups of animals, such as, but not limited to, cotton rats (Sigmodon hispidis, average weight 100 g) cynomolgous macacques (average weight 2.0 kg) are administered the recombinant or chimeric virus or the vaccine of interest or BSA by intramuscular injection, by intravenous injection, or by intranasal route. Prior to, concurrently with, or subsequent to administration of the recombinant virus or the vaccine of the invention, the animals are infected with wild type virus wherein the wild type virus is the virus against which the vaccine was generated. In certain embodiments, the animals are infected with the wild type virus at least 1 day, at least 2 days, at least 3 days, at least 4 days, at least 5 days, at least 6 days, 1 week or 1 or more months subsequent to the administration of the recombinant virus and/or the vaccine of the invention.

After the infection, cotton rats are sacrificed, and their lung tissue is harvested and pulmonary virus titers are determined by plaque titration. Bovine serum albumin (BSA) 10 mg/kg is used as a negative control. Antibody concentrations in the serum at the time of challenge are determined using a sandwich ELISA. Similarly, in macacques, virus titers in nasal and lung lavages can be measured.

(b) Target Populations

In certain embodiments of the invention, the target population for the therapeutic and diagnostic methods of the invention is defined by age. In certain embodiments, the target population for the therapeutic and/or diagnostic methods of the invention is characterized by a disease or disorder in addition to a respiratory tract infection. In a specific embodiment, the target population encompasses young children, below 2 years of age. In a more specific embodiment, the children below the age of 2 years do not suffer from illnesses other than respiratory tract infection. In other embodiments, the target population encompasses patients above 5 years of age. In a more specific embodiment, the patients above the age of 5 years suffer from an additional disease or disorder including cystic fibrosis, leukaemia, and non-Hodgkin lymphoma, or recently received bone marrow or kidney transplantation. In a specific embodiment of the invention, the target population encompasses subjects in which the hMPV infection is associated with immunosuppression of the hosts. In a specific embodiment, the subject is an immunocompromised individual. In certain embodiments, the target population for the methods of the invention encompasses the elderly. In a specific embodiment, the subject to be treated or diagnosed with the methods of the invention was infected with hMPV in the winter months.

(c) Clinical Trials

Vaccines of the invention or fragments thereof tested in in vitro assays and animal models may be further evaluated for safety, tolerance and pharmacokinetics in groups of normal healthy adult volunteers. The volunteers are administered intramuscularly, intravenously or by a pulmonary delivery system a single dose of a recombinant virus of the invention and/or a vaccine of the invention. Each volunteer is monitored at least 24 hours prior to receiving the single dose of the recombinant virus of the invention and/or a vaccine of the invention and each volunteer will be monitored for at least 48 hours after receiving the dose at a clinical site. Then volunteers are monitored as outpatients on days 3, 7, 14, 21, 28, 35, 42, 49, and 56 postdose.

Blood samples are collected via an indwelling catheter or direct venipuncture using 10 ml red-top Vacutainer tubes at the following intervals: (1) prior to administering the dose of the recombinant virus of the invention and/or a vaccine of the invention; (2) during the administration of the dose of the recombinant virus of the invention and/or a vaccine of the invention; (3) 5 minutes, 10 minutes, 15 minutes, 20 minutes, 30 minutes, 1 hour, 2 hours, 4 hours, 8 hours, 12 hours, 24 hours, and 48 hours after administering the dose of the recombinant virus of the invention and/or a vaccine of the invention; and (4) 3 days, 7 days 14 days, 21 days, 28 days, 35 days, 42 days, 49 days, and 56 days after administering the dose of the recombinant virus of the invention and/or a vaccine of the invention. Samples are allowed to clot at room temperature and serum will be collected after centrifugation.

The amount of antibodies generated against the recombinant virus of the invention and/or a vaccine of the invention in the samples from the patients can be quantitated by ELISA. T-cell immunity (cytotoxic and helper responses) in PBMC and lung and nasal lavages can also be monitored.

The concentration of antibody levels in the serum of volunteers are corrected by subtracting the predose serum level (background level) from the serum levels at each collection interval after administration of the dose of recombinant virus of the invention and/or a vaccine of the invention. For each volunteer the pharmacokinetic parameters are computed according to the model-independent approach (Gibaldi et al., eds., 1982, Pharmacokinetics, 2nd edition, Marcel Dekker, New York) from the corrected serum antibody or antibody fragment concentrations.

(d) Methods for Detecting and Diagnosing mMPV

Diagnosis of an mMPV infection, or, more specifically, hMPV infection, can be performed using any method known to the skilled artisan. Descriptions of detection and diagnosis methods for mMPV, such as hMPV, can be found in International Patent Application No PCT/US03/05271 (published as WO 03/072719) and International Patent Application No. PCT/US04/12724 (published as WO 04/096993), both of which are incorporated herein by reference in their entireties. In particular, these international patent application publications describe the detection and diagnosis of mMPV variants A1, A2, B1, and B2.

In general, the virus can detected or diagnosed by virtue of the presence of its components, such as viral protein or viral nucleic acids in a sample (e.g., in the sample from a patient). Such detection is performed using antibodies or nucleic acids that react specifically with these components of mMPV. Alternatively or in addition, antibodies that are formed in a mammal against an mMPV can be detected in a sample from the mammal.

5.11 Compositions of the Invention and Components of Mammalian Metapneumovirus

The invention relates to nucleic acid sequences encoding the mammalian MPV of the invention, proteins of the mutant mMPV, and antibodies against proteins of the mutant mMPV. In particular, the invention provides an mMPV protein carrying one or more of the genetic modifications set forth in section 5.1. The invention also provides nucleic acids encoding these mutated proteins.

In certain embodiments, the invention provides an isolated mammalian MPV protein with an amino acid substitution, deletion, or insertion at one or more amino acid positions selected from the group consisting of: position 66 in the P protein; positions 9, 38, 52, and 132 in the M protein; positions 93, 101, 280, 471, 532, and 538 in the F protein; position 187 in the M2 protein; positions 139 and 164 in the G protein; and positions 235, 323, and 1453 in the L protein, with the proviso that the modification at position 101 in the F protein is not a substitution to Proline and that the modification at position 93 in the F protein is not a substitution to Lysine. In a specific embodiment, the invention provides mMPV L protein with an amino acid substitution, deletion, or insertion at amino acid positions 235 and 323.

In certain embodiments, the isolated mammalian MPV of the invention further comprises a genetic modification that is a silent mutation, i.e., it does not result in an amino acid exchange. In more specific embodiments, the isolated mammalian MPV comprises a silent mutation at one or more of the nucleotide positions selected from the group consisting of: positions 336 and 436 in the M open reading frame.

In another embodiment, the invention provides nucleic acids encoding a protein of mMPV with the following genetic modifications: position 197 in the P open reading frame; position 9, 113, 155, 336, 394, and 436 in the M open reading frame; positions 277, 301, 839, 1412, 1594, and 1612 in the F open reading frame; position 560 in the M2 open reading frame; position 415 and 491 in the G open reading frame; and positions 703, 967, and 4357 in the L open reading frame. In certain embodiments, a nucleotide that is next to one of the recited positions is also mutated resulting in a stabilized codon.

In a further embodiment, the invention provides mMPV proteins with the following genetic modifications: (i) position 66 in the P gene is altered to Val; (ii) position 9 in the M gene is altered to His; (iii) position 38 in the M gene is altered to Ser; (iv) position 52 in the M gene is altered to Pro; (v) position 132 in the M gene is altered to Pro; (vi) position 93 in the F gene is altered to Lys; (vii) position 280 in the F gene is altered to Gly; (viii) position 471 in the F gene is altered to Arg; (ix) position 532 in the F gene is altered to Tyr; (x) position 538 in the F gene is altered to Tyr; (xi) position 187 in the M2 gene is altered to Ile; (xii) position 139 in the G gene is altered to Pro; (xiii) position 164 in the G gene is altered to Pro; (xiv) position 235 in the L gene is altered to Arg; (xv) position 323 in the L gene is altered to Asp; and (xvi) position 1453 in the L gene is altered to Leu.

The invention also provides for vaccines, immunogenic compositions, and pharmaceutical compositions. In certain embodiments, the vaccines, immunogenic compositions, and pharmaceutical compositions comprise the isolated mutated mammalian MPV of the invention and pharmaceutically acceptable excipient. In other embodiments, the vaccines, immunogenic compositions, and pharmaceutical compositions comprise a mutated mMPV protein of the invention and pharmaceutically acceptable excipient.

In another embodiment, the invention provides an isolated chimeric viral RNA polymerase complex comprising RNA polymerase complex subunits from at least two different paramyxoviruses. In an aspect of this embodiment, the RNA polymerase complex subunits are the N, P, L, and M2.1 proteins. In another aspect of this embodiment, the two different paramyxoviruses are selected from the group consisting of RSV, PIV, aMPV, and mammalian MPV.

In certain embodiments, a virus of the invention is inactivated and used for vaccination. In other embodiments, a fragments of a virus of the invention is used for vaccination.

### 6. EXAMPLES

### 6.1 Cold-Passage, Temperature-Sensitive Human Metapneumovirus Vaccines Provide Protective Immunity in Hamsters

Virus adaptation to replication at low temperatures (cold-passage, cp) was used to attenuate hMPV, and the associated sequence-changes in the viral genome were identified. Recombinant viruses containing hMPV or RSV cp-mutations were generated by reverse genetics. These recombinant viruses were found to be temperature-sensitive (ts) in vitro, attenuated for replication in hamsters, yet highly immunogenic in this animal model. Hamsters vaccinated with cp/ts-hMPV strains were protected against heterologous virus infection in the lower respiratory tract (LRT), and had significantly reduced virus titers in the URT. Thus, cp/ts-hMPV represents a promising LAV candidate to protect against hMPV infections.

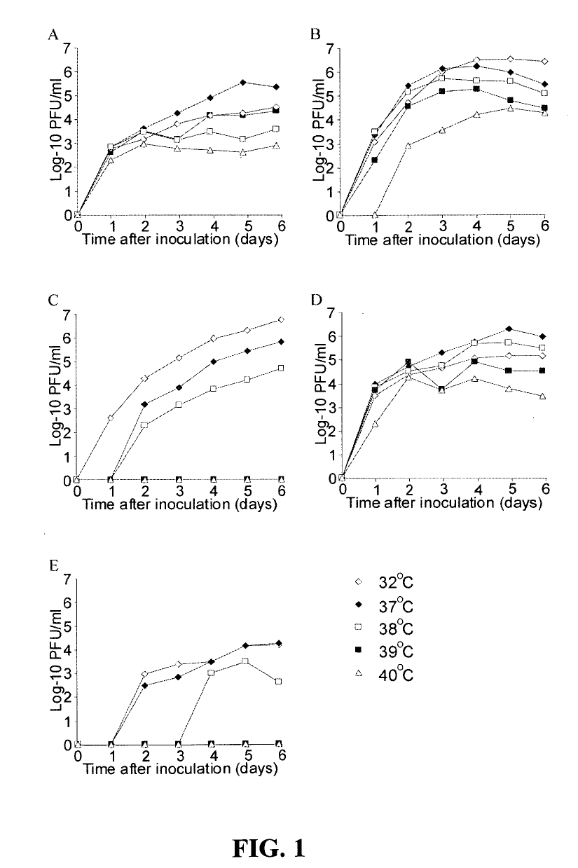

A virus with only 11 of the 19 mutations, hMPV_{M11} turned out to have a ts-phenotype in-vitro (FIG. 1c). Ten of these 11 mutations were non-silent, and were located in the P, M, F, M2, G, and L genes.

Mutations at nt position 3341 (E93K) and 3365 (Sl01P) of the F protein had been described previously (Biacchesi et al., 2006, J Virol 80:5798-806; and Schickli et al., 2005, J Virol 79:10678-89).

(a) Materials and Methods

(i) Cells and Viruses

Vero cells were grown in Iscove's Modified Dulbecco's Medium (IMDM, BioWhittaker, Verviers, Belgium) supplemented with 10% fetal calf serum (FCS, Greiner Bio-One, Alphen aan den Rijn, The Netherlands), 100 IU/ml penicillin, 100 μg/ml streptomycin and 2 mM glutamine. Subclone 83 of WHO Vero cells was selected for virus passaging at low temperatures, and subclone 118 (Kuiken et al., 2004, Am J Pathol 164:1893-900) for all other experiments. To produce purified and concentrated virus stocks, virus strains were grown in infection medium consisting of IMDM supplemented with 4% bovine serum albumin fraction V (Invitrogen, Breda, The Netherlands), 100 IU/ml penicillin, 100 μg/ml streptomycin, 2 mM glutamine and 3.75 μg/ml trypsin until 70-90% of the cells displayed cytopathic effects. After one freeze-thaw cycle, cell-free supernatants were purified and concentrated using a 30-60% (w/w) sucrose gradient.

(ii) Cold-Passaging of Virus

hMPV isolate NL/1/99 (passage 3 at 37° C.) was serially passaged in Vero-83 cells at decreasing temperatures. Virus was cultured at 34° C., 31° C., 28° C. and 25° C. for 3, 3, 2 and 2 passages respectively. When the temperature was decreased further to 22° C. or 20° C., virus replication was seriously impaired, and passaging was thus continued at 25° C. until passage 35 was reached. Cultures were harvested from every passage approximately 7 days after inoculation and stored in 25% sucrose at −70° C.

(iii) Sequence Analysis

Viral RNA was isolated from virus stocks of cp-NL/1/99 passage 35, and intermediate passages 14, 23 and 29, using the High Pure RNA Isolation Kit (Roche Diagnostics, Almere, The Netherlands) according to instructions from the manufacturer. RNA was subsequently used in reverse transcriptase polymerase chain reaction (RT-PCR) assays using primer sets designed on the basis of the full-length genome sequence of NL/1/99 (accession no. AY525843). Both strands of the overlapping PCR-fragments were sequenced without prior cloning, to minimize amplification and sequencing errors. The nucleotide sequence of the cp-NL/1/99 genome was compared with the genome of the wild-type virus to identify nucleotide substitutions. Exemplary sequencing primers are provided as SEQ ID NOs: 140-195.

(iv) Sequence comparison of cold-passaged RSV and hMPV

Genome sequences of RSV strains containing mutations responsible for temperature sensitivity in vitro and attenuation in vivo were aligned with the full-length sequence of hMPV NL/1/99 using BioEdit software (Hall, 1999, Nucleic Acids Symposium Series 41:95-98). Regions containing known ts-mutations in the RSV genome were compared with their counterparts of hMPV, to determine whether RSV ts-mutations could be introduced in the hMPV genome.

(v) Recombinant Viruses

The construction of wild-type recombinant hMPV NL/1/00 and NL/1/99 has been described previously (Herfst et al., 2004, J Virol 78:8264-70). Mutations that were found in cp-NL/1/99, or identified upon sequence comparison of ts-RSV and hMPV, were generated using the QuickChange multi site-directed mutagenesis kit (Stratagene, Leusden, The Netherlands) according to instructions of the manufacturer. Exemplary mutagenic primers are provided as SEQ ID NOs: 123-144.

(vi) Virus Growth at Different Temperatures

To generate virus growth curves, 25 cm² flasks containing confluent Vero-1 18 cells were inoculated at 37° C. for 2 hours with wild-type or mutant hMPV at a multiplicity of infection (MOI) of 0.1. After adsorption of the virus to the cells, the inoculum was removed and cells were washed 2 times with media before addition of 7 ml of fresh media, and incubation at 32° C., 37° C., 38° C., 39° C. or 40° C. Every day, 0.5 ml of the supernatant was collected and replaced by fresh media. To determine viral titers, supernatants were subjected to plaque assays as described previously (Herfst et al., 2004, J Virol 78:8264-70), with the exception that cells were incubated at 32° C. Wild-type NL/1/99 virus and the viruses containing cp-hMPV mutations were incubated for 6 days, whereas the virus harboring the ts-RSV mutations was incubated for 8 days, since only very small plaques were observed after 6 days.

(vii) Hamster Experiments

The replication kinetics and immunogenicity of the recombinant candidate LAVs were studied in Syrian golden hamsters (Mesocricetus auratus; Charles River, Sulzfeld, Germany). Groups of 12 female hamsters, five to seven week old, were inoculated intranasally with 10⁶ 50% tissue-culture infectious dosis (TCID₅₀) of NL/1/99 or LAV in a 100 μl volume. Four days post infection (dpi), lungs and nasal turbinates were collected from six animals per group, snap-frozen immediately and stored at −80° C. until further processing. From the other animals, blood samples were collected by orbital puncture at 21 dpi. Blood samples were stored overnight at room temperature and centrifuged 15 min at 1200×g; serum was collected and stored at −20° C.

For the immunization and challenge experiment, animals were immunized by virus inoculation as described above, with 10⁶ TCID₅₀ of LAV or NL/1/99, or PBS as challenge control. At 21 dpi, animals were challenged intranasally with 10⁷ TCID₅₀ of NL/1/00 virus. Four days after challenge infection, lungs, nasal turbinates and blood samples were collected for further processing.

All intranasal inoculations, orbital punctures and euthanasia were performed under anesthesia with inhaled isoflurane. All animal studies were approved by an independent Animal Ethics Committee and the Dutch authority for working with genetically modified organisms, and were carried out in accordance with animal experimentation guidelines.

(viii) Plaque Reduction Virus Neutralization Assay (PRVN)

Virus neutralizing (VN) antibody titers were determined in serum samples by a plaque reduction virus neutralization (PRVN) assay as described previously (de Graaf et al., 2007, J Virol Methods 143:169-74). Briefly, serum samples were diluted and incubated for 30 minutes at 37° C. with approximately 50 plaque forming units (pfu) of NL/1/00 or NL/1/99, expressing the enhanced green fluorescent protein (eGFP). Subsequently, the virus-serum mixtures were added to Vero-118 cells in 24 well plates, and incubated at 37° C. After two hours, the supernatants were replaced by a mixture of equal amounts of infection medium and 2% methyl cellulose. Six days later, fluorescent plaques were counted using a Typhoon 9410 Variable Mode Imager (GE Healthcare, Diegem, Belgium). VN antibody titers are expressed as the dilution resulting in 50% reduction of the number of plaques, calculated according to the method of Reed and Muench (Reed and Muench, 1938, J Hyg 27:493-497). Per assay, each serum was tested in duplicate against hMPV NL/1/00 and NL/1/99.

(ix) Virus Titrations

Tissues from the inoculated hamsters were homogenized using a Polytron homogenizer (Kinematica AG, Littau-Lucerne, Switzerland) in infection media. After removal of tissue debris by centrifugation, supernatants were used for virus titration in Vero-118 cells. Titrations were performed with 10-fold serial dilutions in 96-well plates (Greiner Bio-One). Conf.luent monolayers of Vero-1 18 cells were spin-inoculated (15 min., 2000×g) with 100 μl of ten fold serial dilutions of each sample and incubated at 37° C. Two hours after the spin-inoculation, the inoculum was replaced with fresh infection media. After 3-4 days, 100 μl of fresh infection media was added to each well. Seven days after inoculation, infected wells were identified by immunofluorescence assays (IFA) with hMPV-specific polyclonal antiserum raised in guinea pigs, as described previously (van den Hoogen et al., 2001, Nat Med 7:719-24). Titers expressed as TCID₅₀ were calculated as described by Reed and Muench (1938, J Hyg 27:493-497). Titers were calculated per gram tissue, with a detection limit of 10^{1.6} and 10^{1.2} TCID₅₀ per gram of tissue for the nasal turbinates and lungs respectively.

(b) Results

(i) Sequence Analysis of cp-NL/1/99

hMPV isolate NL/1/99 was serially passaged in Vero-83 cells at slowly decreasing temperatures until a temperature of 25° C. was reached. When the temperature was further decreased to 22° C. or 20° C., virus replication was severely impaired and virus yield was very poor. Therefore, passaging was continued at 25° C. until passage 35 was reached. Viral RNA of cp-NL/1/99 obtained after 35 passages was subjected to RT-PCR, followed by direct sequencing. Analysis of the full viral genome sequence and comparison with the original NL/1/99 genome revealed the presence of 19 nucleotide changes, resulting in 17 amino acid substitutions (Table 2). Analysis of virus genome sequences after fewer passages (passage 14, 23, and 29) indicated the gradual accumulation of these mutations. One mutation that was found in the L gene after 29 passages had disappeared in the passage 35 virus, but this mutation was also included in further studies. Mutations were found throughout the viral genome in all genes, except the genes encoding the nucleoprotein (N) and the small hydrophobic protein (SH) (Table 2a). Nucleotide substitutions and amino acid substitutions after different number of passages are shown in Table 2b.

TABLE 2a
Nucleotide substitutions found in passage 35 after cold
passaging of hMPV NL/1/99 at 25° C.

| | | | | | | | | | |
|---|---|---|---|---|---|---|---|---|---|
| | | nt | nt | aa | aa | | | | |
| Position^{a} | Gene | (wt) | (cp) | (wt) | (cp) | hMPV_{M19} | hMPV_{M8} | hMPV_{M11} | hMPV_{M2} |

| | | | | | | | | | |
|---|---|---|---|---|---|---|---|---|---|
| 1458 | P | GAA | GTA | Glu | Val | X | | X | |
| 2203 | M | TAT | CAT | Tyr | His | X | | | |
| 2291 | M | TTA | TCA | Leu | Ser | X | | | |
| 2333 | M | CTA | CCA | Leu | Pro | X | | | |
| 2514 | M | GTT | GTG | Val | Val | X | | | |
| 2572 | M | TCA | CCA | Ser | Pro | X | X | X | |
| 2614 | M | CTA | TTA | Leu | Leu | X | X | X | |
| 3341 | F | GAG | AAG | Glu | Lys | X | | | |
| 3365 | F | TCA | CCA | Ser | Pro | X | X | X | |
| 3903 | F | GAT | GGT | Asp | Gly | X | X | X | |
| 4476 | F | CAG | CGG | Gln | Arg | X | X | X | |
| 4658 | F | AAG | TAC | Asn | Tyr | X | | | |
| 4676 | F | CAT | TAT | His | Tyr | X | | | |
| 5255 | M2 | AGC | ATC | Ser | Ile | X | X | X | |
| 6609 | G | ACA | CCA | Thr | Pro | X | X | X | |
| 6685 | G | CAA | CCA | Gln | Pro | X | | | |
| 7826^{b} | L | GGA | AGA | Gly | Arg | X | | X | X |
| 8090 | L | AAC | GAC | Asn | Asp | X | | X | X |
| 11480 | L | TTG | CTC | Phe | Leu | X | X | X | |

^{a}Position is specified as the nucleotide position numbered from the 3′-end of negative sense RNA (accession number AY525843).
^{b}Transient mutation in passage 29 at 25° C.
nt = nucleotide,
wt = wild-type,
cp = cold-passaged,
aa = amino acid,
P = phosphoprotein,
M = matrix protein,
F = fusion protein,
M2 = putative 22K protein,
G = attachment protein,
L = large polymerase protein.
HMPV_{M19} indicates that this virus contains 19 mutations found after cold passaging. X indicates the presence of this mutation in the virus. Nucleotide changes in each codon are underlined.

TABLE 2b
Nucleotide substitutions (nt) and amino acid substitutions (aa) found in passage 14,
23, 29, and 35, respectively, after cold passaging of hMPV NL/1/99 at 25° C.

| | | Nt pos | | | | | | | | | | |
|---|---|---|---|---|---|---|---|---|---|---|---|---|
| | | 26 | 1458 | 2203 | 2291 | 2333 | 2514 | 2572 | 2614 | 3341 | 3365 | 3389 |
| | Gene | Le | P | M | M | M | M | M | M | F | F | F |
| nt | WT | A | A | T | T | T | T | T | C | G | T | G |
| | P14 | G | T | — | — | — | — | — | T | — | C | — |
| | P23 | — | T | — | — | — | — | C | T | — | C | — |
| | P29 | — | T | — | — | — | — | C | T | A | C | T |
| | P35 | — | T | C | C | C | G | C | T | A | C | — |
| aa | WT | — | E | Y | L | L | V | S | L | E | S | A |
| | P14 | — | V | — | — | — | — | — | — | — | P | — |
| | P23 | — | V | — | — | — | — | P | — | — | P | — |
| | P29 | — | V | — | — | — | — | P | — | K | P | S |
| | P35 | — | V | H | S | P | — | P | — | K | P | — |

| | | Nt pos | | | | | | | | | |
|---|---|---|---|---|---|---|---|---|---|---|---|
| | | 3903 | 4476 | 4658 | 4676 | 5255 | 6609 | 6685 | 7826 | 8090 | 11480 |
| | Gene | F | F | F | F | M2 | G | G | L | L | L |
| nt | WT | A | A | A | C | G | A | A | G | A | T |
| | P14 | G | G | — | — | T | C | — | — | G | C |
| | P23 | G | G | — | — | T | C | — | — | G | C |
| | P29 | G | G | — | — | T | C | — | A | G | — |
| | P35 | G | G | T | T | T | C | C | A | G | C |
| aa | WT | D | Q | N | H | S | T | Q | G | N | F |
| | P14 | G | R | — | — | I | P | — | — | D | L |
| | P23 | G | R | — | — | I | P | — | — | D | L |
| | P29 | G | R | — | — | I | P | — | R | D | — |
| | P35 | G | R | Y | Y | I | P | P | R | D | L |

(ii) Sequence Comparison of cp-RSV and hMPV

For RSV, numerous mutations that accumulated in the viral genome after cold-passaging have been identified. After extensive studies, the ts-phenotype of cp-RSV could be assigned to single mutations, or combinations of mutations (Crowe et al., 1995, Vaccine 13:847-55). To explore the possibility of introducing these known cp/ts-mutations of RSV into the hMPV genome, sequences of RSV genes containing known cp/ts-mutations were aligned with their counterparts of hMPV NL/1/99. Most mutations could not be introduced easily in hMPV, because of a lack of similarity between the genes of RSV and hMPV. However, four mutations at position 521 (Crowe et al., 1994, Vaccine 12:691-9), 1169 (Crowe et al., 1995, Vaccine 13:847-55) and 1321 (Crowe et al., 1995, Vaccine 13:847-55) of the L gene and in the gene start (GS) of M2 (Crowe et al., 1994, Vaccine 12:783-90) were identified, for which the hMPV genome was identical to the wild-type RSV sequence (Table 3). Thus, these cp/ts-mutations of RSV could be introduced easily in the genome of hMPV NL/1/99.

TABLE 3
Nucleotide substitutions in cp-RSV that were introduced in
recombinant hMPV/NL/1/99.

| | | | | | | | |
|---|---|---|---|---|---|---|---|
| | | | | nt | aa | aa | |
| Position | Gene | Origin | nt (wt) | (cp) | (wt) | (cp) | Ref. |

| | | | | | | | |
|---|---|---|---|---|---|---|---|
| 521 | L | cpts530 | TTC (RSV) | TTA | Phe | Leu | 5 |
| | | | TTT (hMPV) | | | | |
| 1169 | L | cpts530/1009 | ATG | GTG | Met | Val | 6 |
| 1321 | L | cpts530/1030 | TAT | AAT | Tyr | Asn | 6 |
| — | GS-M2 | cpts248/404 | AATA | AACA | — | — | 4 |

^{a}Position is specified as the amino acid number of the L gene of RSV.
nt = nucleotide,
wt = wild-type,
cp = cold-passaged,
aa = amino acid,
L = large polymerase protein,
GS-M2 = gene-start sequence of the M2 gene. Nucleotides changes in each codon or nucleotide sequence are underlined.

(iii) Construction of Recombinant hMPV cp-NL/1/99

Wild-type recombinant hMPV NL/1/99 was used as a backbone for the introduction of mutations as listed in Tables 2 and 3. Three different viruses containing all mutations or subsets of cp-hMPV mutations were constructed. These viruses containing 19, 8 or 11 nucleotide substitutions were named hMPV_{M19}, hMPV_{M8} and hMPV_{M11} respectively, based on the number of mutations that were introduced (Table 2). Mutant virus hMPV_{M19} could not be rescued by reverse genetics after three attempts. The parental virus obtained after 35 passages at 25° C. also replicated very poorly, to low virus titers. Therefore, we next attempted to rescue recombinant viruses that contained only a selection of the cp-mutations, 8 and 11 respectively, and that were generated as cloning intermediates during the cloning of hMPV_{M19}.

Upon introduction of the four cp-RSV mutations in the NL/1/99 backbone, no virus could be recovered after three attempts. Therefore, four viruses containing each possible combination of three mutations were generated, thus omitting one of the mutations. Only the virus in which the L1321 mutation was left out (named hMPV_{RSV3} hereafter) could be rescued. This cp-RSV mutation thus appeared to be lethal for hMPV.

(iv) Temperature-Sensitivity

To study the possible temperature-sensitive phenotype of recombinant viruses, virus growth curves were generated at different temperatures. Vero cells in 25 cm² flasks were inoculated at an MOI of 0.1, after which the cultures were incubated at 32° C., 37° C., 38° C., 39° C. or 40° C. Plaque assays were performed to determine the viral titers in the supernatants of samples that were collected daily. Wild-type hMPV was able to replicate at all temperatures, with the highest virus titer obtained at 37° C. At 40° C., the virus titer was more than 100-fold reduced compared to the optimal temperature of 37° C. (FIG. 1a). HMPV_{M8} which was an intermediate virus in the cloning procedure of hMPV_{M19}, also replicated at all temperatures, however with higher titers as compared to wild-type hMPV, and an optimal replication temperature of 32° C. (FIG. 1b). Although this virus was not temperature sensitive, it displayed faster replication kinetics in Vero cells and reached high maximum virus titers. Mutant hMPV_{M11} also displayed optimal virus growth at a temperature of 32° C., and virus titers at 6 dpi were even higher as compared to hMPVM₈ (FIG. 1c). This virus did not replicate at 39° C. and 40° C., demonstrating that this virus was temperature-sensitive. The only differences between hMPVM 11 and hMPV_{M8} were two mutations in the L gene and one mutation in the P gene (Table 2). Since for RSV most mutations causing temperature-sensitivity were located in the L gene, hMPV_{M2} was constructed which only had two L mutations (nt 7826 and 8090, Table 2) as compared to wild-type NL/1/99. The replication kinetics of hMPV_{M2} was most similar to that of the wild-type NL/1/99 virus (compare FIGS. 1a and 1d), suggesting that the mutation in the P gene (nt 1458) contributed to the temperature-sensitive phenotype of hMPV_{M11}.

The only viable NL/1/99 with cp-RSV mutations, hMPV_{RSV3}, replicated slowly and to low virus titers at 32° C. and 37° C. At 38° C., no virus was detected until 4 dpi, and at 39° C. and 40° C. the virus did not replicate at all. Thus, hMPV_{RSV3} appeared to be temperature-sensitive in vitro (FIG. 1e).

(v) Replication Kinetics and Immunogenicity in Hamsters

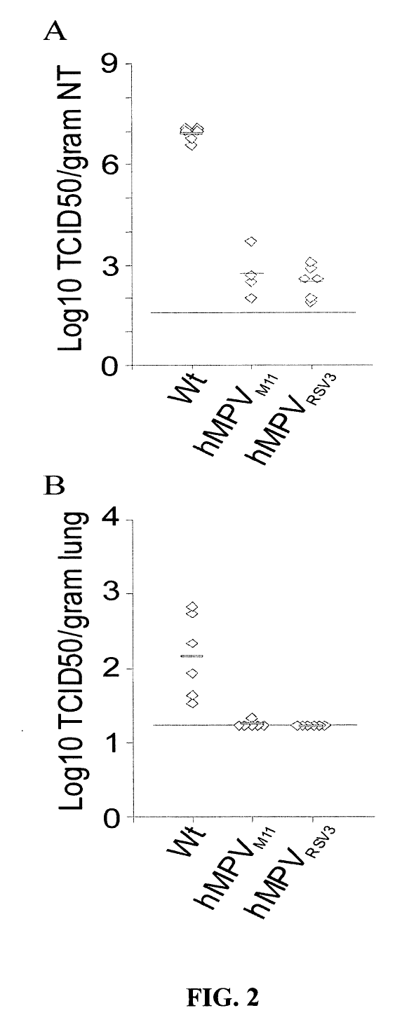

For the two viruses with a temperature-sensitive phenotype in-vitro, hMPV_{M11} and hMPV_{RSV3}, the level of attenuation in hamsters was tested. Syrian golden hamsters were inoculated with hMPV_{M11}, hMPV_{RSV3}, or wild-type NL/1/99 (12 animals per group), after which virus titers in the lungs and NT were compared at four dpi (6 animals per group), and virus neutralizing antibody titers were determined at 21 dpi (6 animals per group). In the NT of animals inoculated with wild-type hMPV, high virus titers up to 10⁷ TCID₅₀/gram NT were detected (FIG. 2a). In the animals inoculated with each of the candidate LAVs however, mean virus titers ranged from 10² and 10⁴ TCID₅₀/gram NT, indicating that virus replication was ˜10.000 fold reduced in the URT. In the lungs of animals inoculated with wild-type hMPV, the mean virus titer was 10^{2.2} TCID₅₀/gram lung, while in the animals inoculated with hMPV_{M11} or hMPV_{RSV3} virus titers were below the detection limit of 10^{1.2} TCID₅₀, with the exception of a single animal in the hMPV_{M11} inoculate group (10^{1.3} TCID₅₀). Thus, both viruses appeared to be highly attenuated in hamsters; virus replication was restricted to the URT, where virus titers were ˜10.000 fold reduced compared to wild-type hMPV.

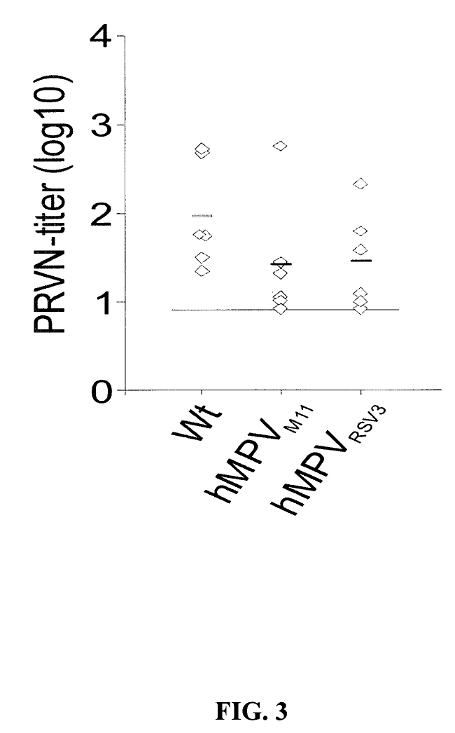

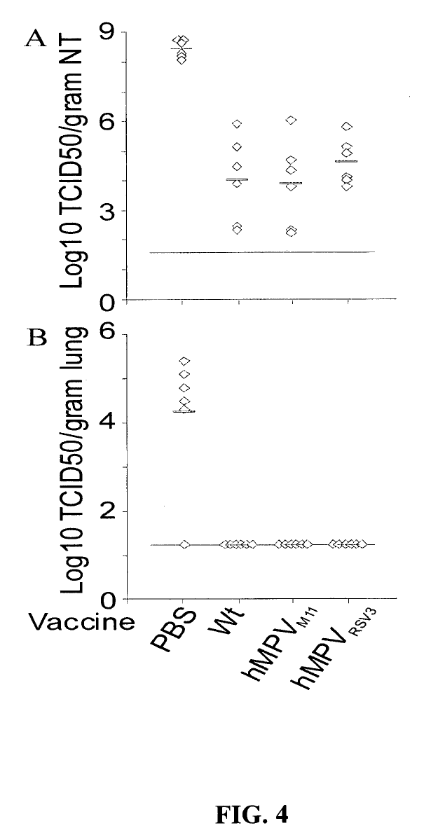

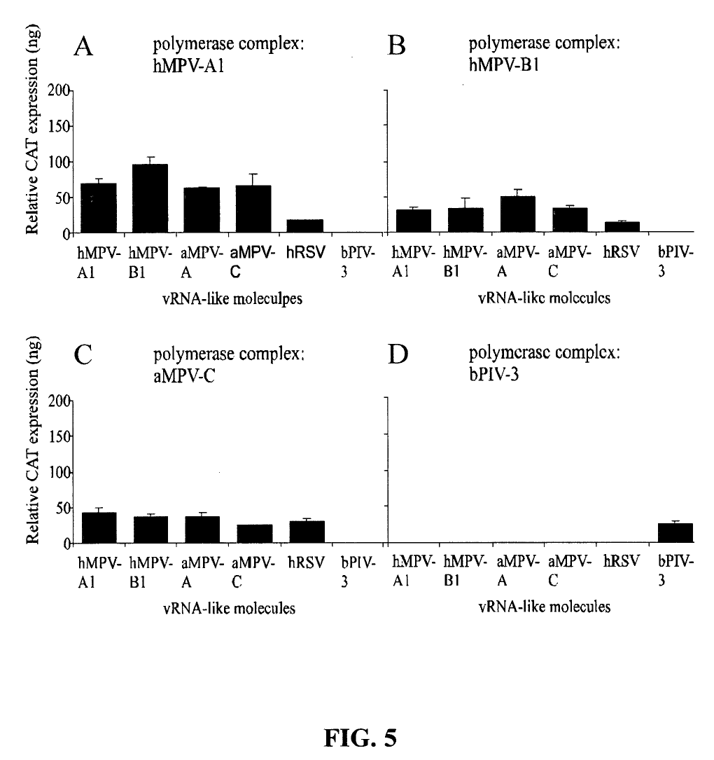

From the remaining six animals of each group, serum samples were collected and subjected to a PRVN assay to determine virus neutralizing antibody titers against hMPV NL/1/99, induced by the candidate LAVs (FIG. 3). The PRVN titers in the wild-type hMPV inoculated animals were slightly higher than those observed in the hMPV_{M11} or hMPV_{RSV3} inoculated animals (mean VN antibody titers of 90, 25, and 28 respectively, not statistically significant, Mann-Whitney test).

(vi) Immunization-Challenge Experiment

Since both hMPV_{M11} and hMPV_{RSV3} induced a detectable but low virus neutralizing antibody response, the induction of protective immunity to prevent subsequent hMPV challenge infection was investigated. Groups of six animals were immunized with 10⁶ TCID₅₀ of hMPV_{M11}, hMPV_{RSV3}, wild-type hMPV NL/1/99 or PBS. Three weeks after immunization, animals were challenged with 10⁷ TCID₅₀ of the heterologous hMPV strain NL/1/00. Four days after challenge infection, lungs, nasal turbinates and blood samples were collected. In PBS-immunized control hamsters, virus titers upon challenge reached >10⁸ TCID₅₀/gram tissue in the NT samples. These virus titers were more than 1.000-fold reduced in animals immunized with hMPV_{RSV3}, and >10.000-fold reduced in the animals immunized with hMPV_{M11} or wild-type hMPV. In the lungs of PBS-immunized animals, the mean virus titers after challenge infection was 10^{4.3} TCID₅₀/gram lung tissue. Virus was undetectable in all animals immunized with hMPV_{M11}, hMPV_{RSV3}, and wild-type hMPV NL/1/99 (Mann-Whitney test, P<0.05). Thus, hMPV_{M11} and hMPV_{RSV3} are attenuated in hamsters, yet induce an hMPV-specific immune response which is sufficient to provide protective immunity to prevent hMPV lower respiratory tract infection.

Vaccinated animals were completely protected from hMPV LRT infection, and virus titers in the URT were reduced to the same extend as seen in hamsters exposed to wild-type hMPV.

Our results demonstrate that immunization of Syrian golden hamsters with attenuated recombinant viruses containing cp-hMPV or cp-RSV mutations, induced a good antibody response, and provided complete protection against LRT infection with heterologous virus.

### 6.2 Specificity and Functional Interaction of the Polymerase Complex Proteins of Human and Avian Metapneumoviruses

(a) Introduction

A tool frequently used for the analysis of cis- and trans-acting elements influencing viral RNA synthesis are minireplicon systems. In such systems all components of the viral polymerase complex are transfected and the replication and transcription of a synthetic vRNA-like molecule is measured using reporter genes. For the genera respirovirus, henipahvirus, and pneumovirus of the paramyxovirus family it was shown that polymerase complexes provided by expression plasmids or co-infection could replicate vRNA-like molecules of other viruses belonging to the same genus (Halpin et al., 2004, J Gen Virol 85, 701-7; Pelet et al., 1996, J Gen Virol 77 (Pt 10), 2465-9; Yunus et al., 1999, Arch Virol 144, 1977-90). VRNA-like molecules of morbilliviruses are efficiently replicated by polymerase complex proteins of other morbilliviruses but not or less efficiently by polymerase complexes consisting of proteins of two different morbilliviruses (Bailey et al., 2007, Virus Res. 126:250-5; Brown et al., 2005, J Gen Virol 86, 1077-81). For pneumoviruses it was shown that vRNA-like molecules based on aMPV-A were replicated by the polymerase complex proteins of hRSV (Marriott et al., 2001, J Virol 75, 6265-72). For metapneumoviruses it has been shown that polymerase complexes consisting of both human and avian metapneumovirus components are able to rescue virus from cDNA (Govindarajan et al., 2006, Virus Genes 30, 331-3).

Chimeric viruses in which polymerase genes are exchanged between two related viruses are frequently used to generate attenuated vaccine strains (Bailly et al., 2000, J Virol 74, 3188-95; Govindarajan et al., 2006, Virus Genes 30, 331-3; Pham et al., 2005, J Virol 79, 15114-22; Skiadopoulos et al., 2003, J Virol 77, 1141-8).

An aMPV-C minireplicon system was generated and used in combination with minireplicon systems for hMPV-A1 and hMPV-B1. Each of these sets of metapneumovirus polymerase complex proteins was able to replicate synthetic vRNA-like molecules of hMPV-A1 and B1, aMPV-A and C and hRSV but not human parainfluenza virus type 3 (bPIV-3). To test the functional interaction of polymerase complex proteins of hMPV-A 1, B1 and aMPV-C, vRNA-like molecules were co-transfected with different combinations of N, P, L and M2.1 expression plasmids revealing that chimeric polymerase complexes were functional but with different efficiencies. Subsequently, several chimeric viruses were created which contained polymerase complex genes of hMPV-A 1 and B1 or hMPV-B1 and aMPV-C. Most of these chimeric viruses replicated with similar efficiency as the wild type viruses in vitro. A subset of these was tested for attenuation in hamsters and replicated to lower titers than the wild type viruses. This study provides insight in the specificity and functional interaction of polymerase complex proteins of human and avian metapneumoviruses.

Previously it has been shown that hRSV and aMPV-A vRNA-like molecules can be replicated upon heterologous or homologous infection with aMPV-A or hRSV or co-transfection with hRSV polymerase protein expression plasmids (Marriott et al., 2001, J Virol 75, 6265-72). Chimeric polymerase complexes of members of the Paramyxoviridae family vary in their ability to replicate vRNA-like molecules or rescue recombinant virus (Bailey et al., 2007, Virus Res. 126:250-5; Brown et al., 2005, J Gen Virol 86, 1077-81; Govindarajan et al., 2006, J Virol 80, 5790-7). Exchanging polymerase genes between two related viruses with different host range is a method frequently used for the design of live attenuated vaccine strains (Bailly et al., 2000, J Virol 74, 3188-95; Govindarajan et al., 2006, J Virol 80, 5790-7; Pham et al., 2005, J Virol 79, 15114-22; Skiadopoulos et al., 2003, J Virol 77, 1141-8).

Exchanging genes between two related paramyxoviruses with different host range or replication properties has been shown useful for the rational design of live attenuated vaccine strains (Bailly et al., 2000, J Virol 74, 3188-95; Govindarajan et al., 2006, J Virol 80, 5790-7; Pham et al., 2005, J Virol 79, 15114-22; Skiadopoulos et al., 2003, J Virol 77, 1141-8).

(b) Materials and Methods

(i) Cells, Media and Viruses

Vero-1 18 (Kuiken et al., 2004, Am J Pathol 164, 1893-900) cells were cultured in Iscove's Modified Dulbecco's medium (BioWhittaker, Verviers, Belgium) supplemented with 10% FCS, 100 IU of penicillin/ml, 100 μg of streptomycin/ml, and 2 mM glutamine as described previously. For hMPV rescue, Vero-118 cells and BSR-T7 cells were co-cultured in Dulbecco's Modified Eagle medium supplemented with 3% Fetal Calf Serum (FCS), 100 IU of penicillin/ml, 100 μg of streptomycin/ml, 2 mM glutamine, and 0.25 mg of trypsin/ml. For virus propagation and titration of hMPV-A1 and B1, all chimeric viruses, and aMPV-C (Colorado strain, Intervet, Boxmeer, The Netherlands), Vero-1 18 cells were grown in Iscove's Modified Dulbecco's medium supplemented with 4% bovine serum albumin fraction V (Invitrogen, Breda, the Netherlands), 100 IU of penicillin, 2 mM glutamine, and 3.75 μg of trypsin. Baby hamster kidney cells stably expressing T7 RNA polymerase (BSR-T7, (Buchholz et al., 1999, J Virol 73, 251-9)) were grown in Dulbecco's Modified Eagle medium (BioWhittaker, Verviers, Belgium) supplemented with 10% FCS, nonessential amino acids, 100 IU of Penicillin/ml, 100 μg of streptomycin/ml, 2 mM glutamine and supplemented with 0.5 mg of G418 (Life Technologies, Breda, The Netherlands).

(ii) Plasmid Construction.

(A) Minireplicon Systems.

The minireplicon systems of hMPV-A1 and B1 have been described previously (Herfst et al., 2004, J Virol 78, 8264-70). The minireplicon system for aMPV-C was constructed using the same vectors, with primers designed on the basis of the published sequence of aMPV-C (Gene bank accession no. AY57978). For the construction of the aMPV-C vRNA-like molecule, the leader and the GS of N and the trailer and GE of L were amplified by PCR and ligated, separated by two BsmBI sites. This fragment was ligated in a plasmid containing T7 RNA polymerase promoter (P_{T7}) and terminator (T_{T7}) sequences and a hepatitis delta ribozyme (pSP72-P_{T7}-δ-T_{T7}, (Herfst et al., 2004, J Virol 78, 8264-70) to yield pSP72-PT₇-Tr-Le-δ-T_{T7}. The ORF of CAT was amplified by PCR and cloned in the BsmBI sites between the GS of N and GE of L to yield pSP72-P_{T7}-Tr-CAT-Le-δ-T_{T7}. For the construction of plasmids expressing the polymerase complex proteins, the N, P, and M2.1 ORFs of aMPV-C were amplified by PCR using primers spanning the start and stop codons and flanked by NcoI and XhoI sites, respectively, and were cloned in the multiple cloning site of pCITE (Novagen) to yield plasmids pCITE-N, pCITE-P, and pCITE-M2.1. Constructs encoding the L gene of aMPV-C were assembled from overlapping PCR fragments using restriction sites in the L gene and were cloned in pCITE. The restriction sites used were NcoI (introduced at nt 6935 before the start codon of L), ScaI (nt 8557), NdeI (nt 9770), and BclI (nt 11535) and XhoI (introduced at nt 13135 after the trailer). The minireplicon system of aMPV-A was a kind gift of Dr A. Easton. The minireplicon systems of bPIV-3 and hRSV are published in Jin et al., 1998, Virology 251, 206-14.

(B) Full-Length cDNA Vectors

The full-length hMPV cDNA plasmids for hMPV-A1 and B1 have been described previously (Herfst et al., 2004, J Virol 78, 8264-70). For the construction of the full-length chimeric hMPV-B1 cDNA plasmids containing the N,N and P, P, M2.1 and L of hMPV-A1 or the N, P or L of aMPV-C cDNA, fragments of hMPV-B1 were amplified by PCR and cloned in pCR4TOPO (Invitrogen, Breda, the Netherlands). All fragment were cloned such that type II restriction sites replaced the N, P, M2.1 or L ORFs and their GS and GE sequences. The N and P ORFs of aMPV-C and the N,N and P, P and M2.1 ORFs of hMPV-A1 were amplified by PCR using primers spanning GS and GE flanked by type II restriction sites. The L ORF and GS and GE of hMPV-A1 was assembled from overlapping PCR fragments using unique restriction sites in the L ORF and type II sites flanking GS and GE. For the construction of full-length chimeric hMPV-B1 cDNA plasmid containing the L of aMPV-C, fragments of hMPV-B1 were amplified by PCR and cloned in pBluescript SK+ (Stratagene). The L ORF of aMPV-C was assembled from overlapping PCR fragments using unique restriction sites in the L ORF and type II sites flanking GS and GE were introduced. Using unique restriction sites the fragments containing the desired ORF were swapped back into the full-length hMPV-B1 cDNA plasmids. All plasmid inserts were sequenced to ensure the absence of mutations.

(iii) Minireplicon Assays.

BSR-T7 cells grown to 80-95% confluence in six-well plates were transfected with 1 μg of the vector expressing the vRNA-like molecule, 1 μg pCITE-N, 0.5 μg pCITE-P, 0.5 μg pCITE-L, 0.5 μg pCITE-M2.1 and 0.4 μg of pTS27, a vector expressing β-galactosidase under the control of a cytomegalovirus immediate-early (CMV IE) promoter (a kind gift of Dr M. Malim). Cells were analyzed 3 days after transfection by using enzyme-linked immunosorbent assays (ELISA) for CAT and β-galactosidase (Roche Diagnostics, Almere, the Netherlands) according to the instructions from the manufacturer. All transfections were done in triplo and CAT values were standardized to 10 ng β-galactosidase to control for transfection efficiency and sample processing.

(iv) Recovery of Recombinant hMPV

Recovery of recombinant hMPV was performed as described previously (Herfst et al., 2004, J Virol 78, 8264-70). Briefly, BSR-T7 cells were transfected for 5 hours with 5 μg of the full-length hMPV cDNA plasmid, 2 μg pCITE-N, 2 μg pCITE-P, 1 μg pCITE-L and 1 μg pCITE-M2.1 using Lipofectamine 2000 (Invitrogen, Breda, the Netherlands). The hMPV-B1 polymerase expression plasmid set was used for the recovery of all chimeric hMPV-B1/hMPV-A1 and hMPV-B1/aMPV-C viruses. After transfection, the media was replaced with fresh media supplemented with trypsin. Three days after transfection, the BSR-T7 cells were scraped and cocultured with Vero-118 cells for 8 days.

(v) Virus Titrations

Viruses were propagated in Vero-118 cells and virus titers were determined as described previously (Herfst et al., 2004, J Virol 78, 8264-70). Conf.luent monolayers of Vero-118 cells in 96-well plates (Greiner Bio-One) were spin-inoculated (15 min., 2000×g) with 100 μl of ten fold serial dilutions of each sample and incubated at 37° C. After 2 hours and again after 3-4 days, the inoculum was replaced with fresh infection media. Seven days after inoculation, infected wells were identified by immunofluorescence assays (IFA) with hMPV-specific polyclonal antiserum raised in guinea pigs, as described previously (van den Hoogen et al., 2001, Nat Med 7, 719-24). Titers expressed as 50% tissue culture infectious dose (TCID₅₀) were calculated as described by Reed and Muench (Reed & Muench, 1938, J Hyg 27, 493-497).

(vi) Growth Curves

Growth curves were generated as described previously (Herfst et al., 2004, J Virol 78, 8264-70) 25-cm² flasks containing confluent Vero-118 cells were inoculated at 37° C. for 2 h with hMPVAI and B1, aMPV-C or one of the chimeric virus strains at a multiplicity of infection of 0.1. After adsorption of the virus to the cells, the inoculum was removed and cells were washed two times with media before addition of 7 ml of fresh media and incubation at 37° C. Every day, 0.5 ml of supernatant was collected and replaced by fresh media. Plaque assays were performed to determine viral titers.

(vii) Plaque Assays

Plaque assays were performed as described previously (Herfst et al., 2004, J Virol 78, 8264-70), with minor adjustments. Twenty-four-well plates containing 95% confluent monolayers of Vero-118 cells were inoculated with 10-fold serial virus dilutions for 1 h at 37° C., after which the media was replaced by 0.5 ml of fresh media and 0.5 ml of 2% methyl cellulose (MSD, Haarlem, the Netherlands) and cells were incubated at 37° C. for 4 days. Methyl cellulose overlays were removed and cells were fixed with 80% acetone. Cells were incubated with hMPV-specific polyclonal antiserum for 1 h at 37° C., followed by incubation with horseradish peroxidase-labeled rabbit anti-guinea pig antibodies (DakoCytomation, Heverlee, Belgium). Positive plaques were counted after incubation with a freshly prepared solution of 3-amino-9-ethylcarbazole (AEC) substrate chromogen (Sigma-Aldrich, Buchs, Switzerland) to determine viral titers.

(viii) Hamster Experiments

Six-week-old female Syrian golden hamsters (Mesocricetus auratus) (Harlan Sprague Dawley Inq., Horst, The Netherlands) were inoculated intranasally with 10⁶ TCID₅₀ of virus in 100 μl, diluted in PBS. Four days after inoculation, lungs and nasal turbinates (NT) were collected, snap-frozen immediately and stored at −80° C. until further processing. All intranasal inoculations and euthanasia were performed under anesthesia with inhaled isoflurane. All animal studies were approved by the Animal Ethics Committee and the Dutch authority for working with genetically modified organisms, and were carried out in accordance with animal experimentation guidelines. Tissues from the inoculated hamsters were homogenized using a Polytron homogenizer (Kinematica AG, Littau-Luceme, Switzerland) in infection media. After removal of tissue debris by centrifugation, supernatants were used for virus titration in Vero-118 cells. Titers were calculated per gram tissue, with a detection limit of 10^{1.6} and 10^{1.2} TCID₅₀ per gram of tissue for NT and lung samples respectively.

(c) Results

(i) Replication of Paramyxovirus vRNA-Like Molecules by Heterologous Polymerase Complexes

To determine whether the polymerase complexes of different members of the paramyxovirus family can recognize heterologous templates, vRNA-like molecules that contained a CAT ORF in antisense orientation flanked by the genomic termini of hMPV-A1 and B1, aMPV-A and C, hRSV and bPIV-3 were used. Each of these plasmids was co-transfected in BSR-T7 cells with four plasmids expressing the N, P, L, and M2.1 proteins of hMPV-A1, B1, or aMPV-C. For the bPIV-3 system the M2.1 expression plasmid was omitted as the virus does not need M2.1 for efficient replication and transcription (Durbin et al., 1997, Virology 234, 74-83). Upon cotransfection of plasmids expressing the N, P, L and M2.1 protein together with their homologous vRNA-like molecules, the reporter gene CAT was expressed efficiently (FIG. 5). Polymerase complex proteins of hMPV-A1 and B1 and aMPV-C could replicate the vRNA-like molecules of hMPV-A1 and B1, aMPV-A and C, and hRSV but not bPIV-3. Conversely, the bPIV-3 polymerase complex only replicated the homologous vRNA-like molecule. The metapneumovirus polymerase complexes revealed little substrate specificity as they replicated heterologous metapneumovirus vRNA-like molecules with similar efficiency as homologous molecules. VRNA-like molecules based on the hRSV genome were replicated less efficiently than the metapneumovirus vRNA-like molecules by the human metapneumoviruses polymerase complexes.

(ii) Replication of Metapneumovirus vRNA-Like Molecules by Chimeric Polymerase complexes

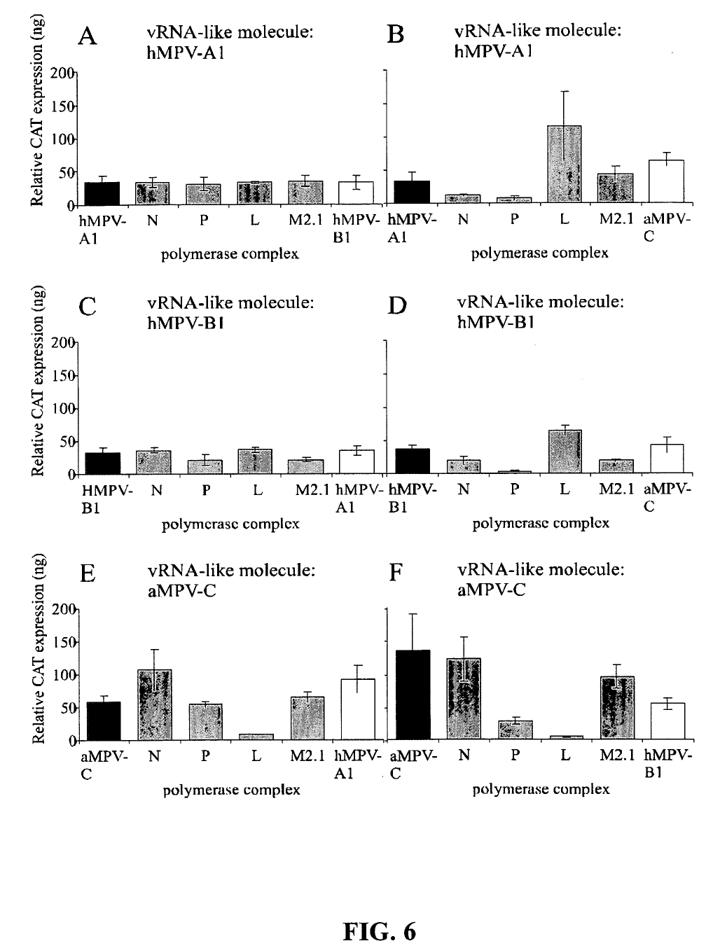

For morbilliviruses it was found that the vRNA-like molecules can be replicated by heterologous polymerase complexes but not or less efficiently by chimeric polymerase complexes (Bailey et al., 2007, Virus Res. 126:250-5; Brown et al., 2005, J Gen Virol 86, 1077-81). To investigate the functional interaction between polymerase complex proteins of human and avian metapneumoviruses, the N, P, L and M2.1 expression plasmids were individually exchanged between the hMPV-A1 and B1 and aMPV-C minireplicon systems (FIG. 6). All chimeric hMPV-A1/B1 polymerase complexes were functional and replicated vRNA-like molecules equally efficient as the homologous complex protein sets (FIGS. 6A and C). Chimeric polymerase complexes consisting of hMPV-A1 and aMPV-C or hMPV-B1 and aMPV-C components were functional but differed in their replication efficiency (6B, D-F). Furthermore hMPV-A1 and hMPV-B1 polymerase complex proteins appeared to be highly conserved as they caused similar increases and decreases in replication efficiency when exchanged with those of aMPV-C (compare FIGS. 6B and 6D or 6E and 6F). Chimeric hMPV-A1 (FIG. 6B) and hMPV-B1 (FIG. 6D) polymerase complexes in which the P protein was substituted with P of aMPV-C were less efficient in the replication of vRNA-like molecules than the wild-type hMPV polymerase complexes. Smaller differences were observed when the N or M2.1 proteins were substituted. Chimeric polymerase complexes in which the hMPV-A1 or B1 L protein was substituted with the L protein of aMPV-C replicated hMPV-A1 or B1 vRNA-like molecules with higher efficiency compared to polymerase complexes consisting of hMPV-A1 or B1 or aMPV-C proteins only (FIG. 6B, 6D). Chimeric polymerase complexes in which the aMPV-C L protein was substituted with the L protein of hMPV-A1 or B1 replicated aMPV-C vRNA-like molecules with lower efficiency compared to polymerase complexes consisting of hMPV-A1 or B1 or aMPV-C proteins only (FIG. 6E, 6F). Substitution of the N or M2.1 proteins had less of an impact on replication efficiency. It should be noted that the M2.1 expression plasmid of pneumovirus and metapneumovirus minireplicon systems can be omitted, without significant effects on the levels of CAT (Collins et al., 1995, Proc Natl Acad Sci USA 92, 11563-7; Collins et al., 1996, Proc Natl Acad Sci USA 93, 81-5; Herfst et al., 2004, J Virol 78, 8264-70; Naylor et al., 2004, J Gen Virol 85, 3219-27).

(iii) Rescue of hMPVB1 by Chimeric Polymerase Complexes.

The ability to rescue recombinant hMPV using these chimeric polymerase complexes was tested. The full-length hMPV-B1 cDNA plasmid was co-transfected into BSR-T7 cells with the N, P, L and M2.1 expression plasmids of hMPV-B1 or aMPV-C or sets in which the hMPV-B1 N, P, L and M2.1 expression plasmids were individually exchanged with those of aMPV-C. It was possible to rescue hMPV-B1 by the hMPV-B1, aMPV-C and all chimeric hMPV-B1/aMPV-C polymerase complexes without significant differences in efficiency.

(iv) Growth of Chimeric hMPV-B1/hMPV-A1 Viruses in Tissue Culture

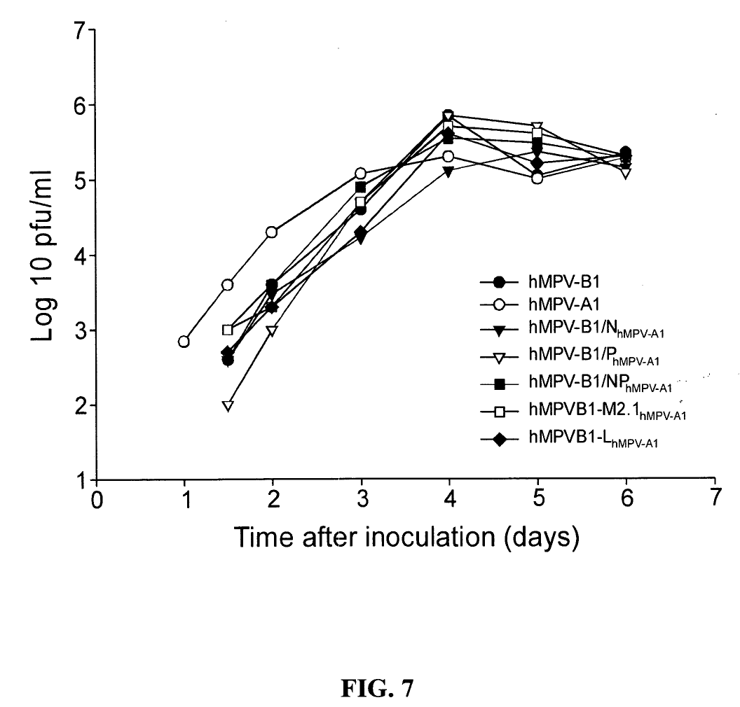

Minireplicon systems only include the components of the viral polymerase complex necessary for replication and transcription of the viral genome. To investigate the functionality of chimeric polymerase complexes in the context of a complete virus, a panel of chimeric viruses was made. The N, P, N and P, M2.1 and L genes of hMPV-B1 were replaced with those of hMPV-A1 resulting in hMPV-B1/N_{hMPV-A1}, hMPV-B1/P_{hMPV-A1}, hMPV-B1/NP_{hMPV-A1}, hMPV-B1/M2.1_{hMPV-A1} and hMPV-B1/L_{hMPV-A1} respectively. Standard multi-step growth curves were generated to compare the growth of the chimeric viruses with those of the parental viruses hMPV-A1 and B1. Vero-118 cells were infected at a multiplicity of infection (MOI) of 0.1 with the parental and chimeric viruses after which supernatant samples were collected daily and virus titers were determined by plaque assay (FIG. 7). No apparent differences in replication kinetics could be observed, indicating that the viruses containing chimeric polymerase complexes are fully functional in vitro, in agreement with the minireplicon assays.

(v) Replication Characteristics of Chimeric hMPV-B1/aMPV-C Viruses in Tissue Culture

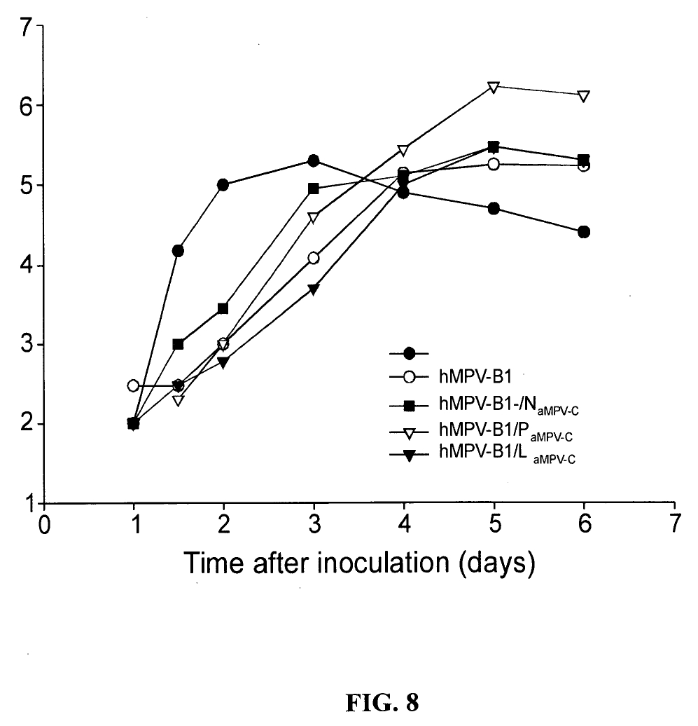

To further investigate the functionality of chimeric hMPV-B1/aMPV-C polymerase complexes a panel of chimeric viruses was made. The N, P and L genes of hMPV-B1 were replaced with those of aMPV-C resulting in hMPV-B1/N_{aMPV-C}, hMPV-B1/P_{aMPV-C} and hMPV-B1/L_{aMPV-C} respectively. All chimeras could be rescued with similar efficiency as hMPV-B1. Standard multi-step growth curves were generated to compare the growth of the chimeric viruses with those of the parental viruses hMPV-B1 and aMPV-C. Vero-118 cells were infected at a MOI of 0.1 with parental and chimeric viruses after which supernatants were collected daily and virus titers were determined by plaque assay (FIG. 8). This revealed that aMPV-C replicated faster than its human counterpart hMPV-B1. Furthermore, hMPV-B1/L_{aMPV-C} and hMPV-B1/N_{aMPV-C} grew to similar titers as the backbone virus hMPV-B1. In contrast the hMPV-B1/P_{aMPV-C} grew to higher titers than hMPV-B1.

(vi) Characterization of Chimeric hMPV-B1/aMPV-C Viruses in Hamsters

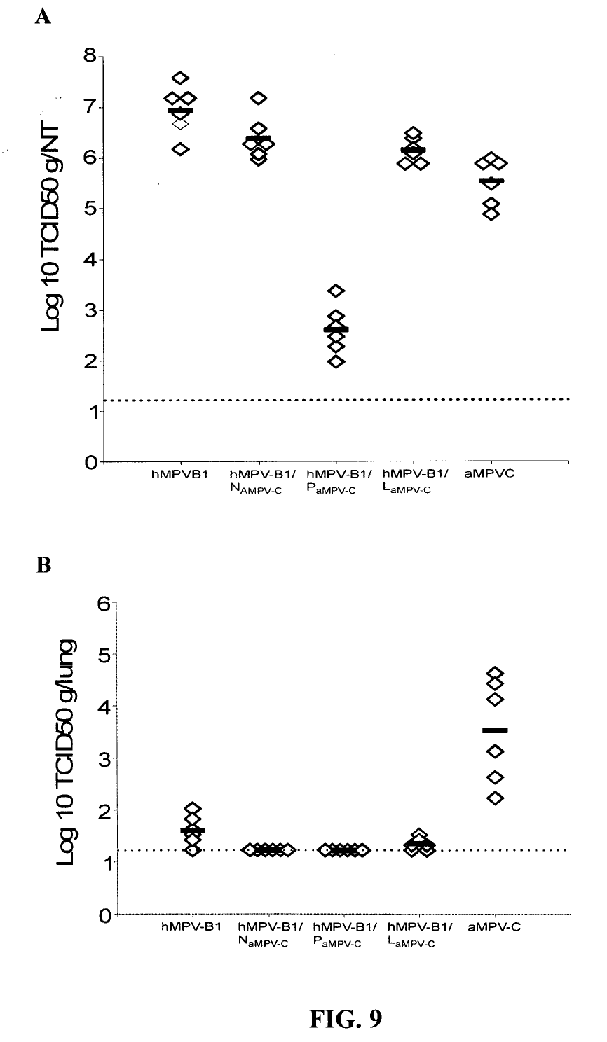

The level of replication of the chimeric hMPV-B1/aMPV-C viruses in the upper and lower respiratory tract were evaluated in Syrian golden hamsters, which represent a permissive small animal model for human Metapneumovirus (MacPhail et al., 2004, J Gen Virol 85, 1655-63). Five groups (n=6) of hamsters were inoculated intranasally with 10⁶ TCID50, the lungs and NT were harvested on day four post-infection, and the titer of virus present in tissue homogenates was determined (FIG. 9). AMPV-C replicated to 100-fold higher titers in the lungs, but 10-fold lower titers in the NT compared to hMPV-B1. The hMPVB1-N_{aMPV-C} and hMPV-B1/L_{aMPV-C} chimeric viruses did not replicate in the lungs and slightly less efficiently in the NT compared to hMPV-B1. HMPV-B 1/P_{aMPV-C} does not replicate in the lungs and resulted in 10.000-fold lower titers in the NT compared to hMPV-B1.

(d) Discussion

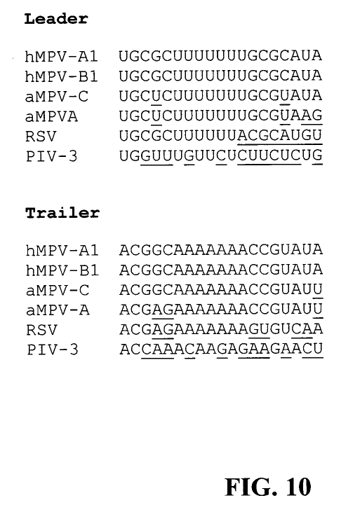

The presented results demonstrate that the cis-acting elements in the genomic termini of hMPV-A1 and B1, aMPV-A and C and hRSV are conserved and functionally interchangeable. Consistently, the Pneumovirus subfamily display a high degree of sequence conservation, but less so between pneumoviruses and bPIV-3 (FIG. 10).

### 6.3 Subunit Vacclnes

The F proteins from hMPV strains NL/1/99 and NL/1/00 were used to vaccinate

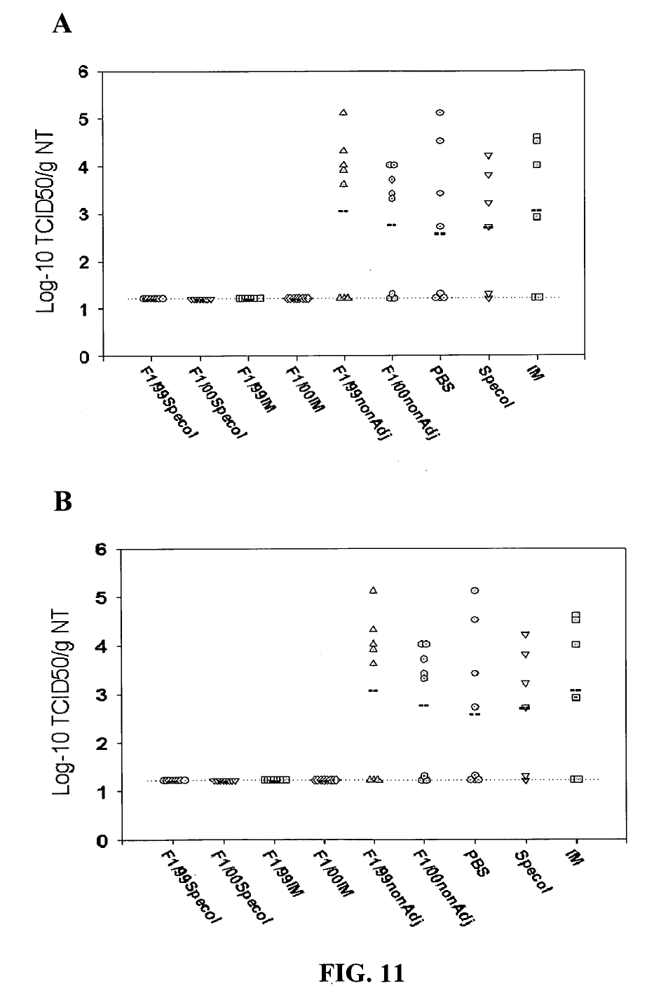

Syrian Golden Hamsters and to determine whether the proteins themselves might work as subunit vaccines, inducing protective immunity to prevent subsequent hMPV challenge infection. Animals were inoculated with 10 μg of the F protein from NL/1/99 or NL/1/00 in the presence or absence of adjuvant. The animals were immunized twice, with a 3 week interval between immunizations. Three weeks after immunization, the animals were challenged with 10⁶ TCID₅₀ of hMPV strain NL/1/00. Four days after the challenge infection, the animals were sacrificed, and the nasal turbinates and lungs were isolated and quantified for hMPV titers by virus titration on Vero cells. Titers of virus in the lungs of animals that were vaccinated with the F protein from hMPV strain NL/1/99 or NL/1/00 were substantially less than titers observed in animals immunized with PBS or adjuvant alone (FIG. 11A). Nasal turbinate titers of virus were slightly less in animals immunized with the F protein from hMPV strain NL/1/99 or NL/1/00 than with PBS or adjuvant alone (SV FIG. 11B).

Plaque reduction virus neutralization assays were performed generally as described in section 6.1 (a)(viii). Virus neutralizing antibody titers were high in sera obtained from animals that were vaccinated with the F protein from hMPV strain NL/1/99 or NL/1/00 in the presence of adjuvant, as evidenced by the high dilutions possible to cause a 50% reduction in the number of plaques formed in the assay (Table 4). Antibodies generated following vaccination with the F protein from hMPV NL/1/99 were effective in reducing the number of plaques from the strain NL/1/00, although were much more effective at neutralizing the parent strain. Similarly, antibodies generated following vaccination with the F protein from hMPV NL/1/00 were capable of reducing plaque formation by the strain NL/1/99, but much more capable of neutralizing NL/1/00 (Table 4).

TABLE 4
Plaque reduction virus neutralization assays

| | | | | | |
|---|---|---|---|---|---|
| F1/99 | F1/00 | F1/99 | F1/00 | F1/99 | F1/00 |
| Spec | Spec | IM | IM | nonA | nonA |

| | | | | | | |
|---|---|---|---|---|---|---|
| 1/00(A) | 230 | 3563 | 163 | 2363 | 9 | 53 |
| 1/99(B) | 4546 | 1044 | 4816 | 963 | 9 | 11 |
| Ratio A-B | | 3.4 | | 2.5 | | 4.8 |
| Ratio B-A | 20 | | 30 | | 1 | |

Mean PRVN titers (8 animals/group), homologous titers are underlined

### 6.4 Nucleotide and Amino Acid Mutations in NL/1/94, NL/17/00, and NL/1/00

hMPV strains NL/1/94 (passage 3 at 37° C.), NL/17/00 (passage 3 at 37° C.), and NL/1/00 (passage 10 at 37° C.) were cold-passaged and analyzed as described in sections 6.1(a)(ii) and 6.1(a)(iii). Briefly, virus was serially passaged in Vero-83 cells at 34° C., 31° C., 28° C. and 25° C. for 3, 3, 2 and 2 passages respectively. When the temperature was decreased further to 22° C. or 20° C., virus replication was seriously impaired, and passaging was thus continued at 25° C. until passage 35 was reached.

Analysis of the full viral genome sequence of hMPV NL/1/94 (B2) after 35 passages and comparison with the original NL/1/94 genome revealed the presence of 27 nucleotide changes, resulting in 17 amino acid substitutions (Table 5). Analysis of virus genome sequences after fewer passages (passage 18 and 29) indicated the gradual accumulation of these mutations.

hMPV strain NL/17/00 (A2) accumulated 9 nucleotide changes by passage 35 as well as a deletion of the nucleotide at position 4692 of the original NL/17/00 genome. These nucleotide changes corresponded to 4 amino acid substitutions (Table 6). Analysis of virus genome sequences after fewer passages (passage 29 and 35) indicated the gradual accumulation of these mutations.

Following passage 35, hMPV strain NL/1/00 (A1) had 11 nucleotide changes corresponding to changes in 8 amino acids (Table 7). Although analysis of the viral genome sequences at passages 14 and 29 indicated the gradual accumulation of these mutations, passage 29 revealed mutations at nucleotide positions 3344, 10598, and 13306 that were not present at passage 35. Furthermore, passages 14 and 35 revealed a nucleotide mutation at nucleotide position 2568 that was not present at passage 29 (Table 7).

TABLE 5
Overview of mutations in NL/1/94 after cold-passaging

| | | Nt pos | | | | | | | | | | | | | |
|---|---|---|---|---|---|---|---|---|---|---|---|---|---|---|---|
| | | 2564 | 3450 | 3755 | 3944 | 3984 | 4487 | 4526 | 5570 | 6072 | 6076 | 6549 | 6608 | 6742 | 8318 |
| Gene | | M | F | F | F | F | F | F | SH | GE-SH | GE-SH | G | G | G | L |
| nt | WT | T | G | G | G | A | G | A | A | C | C | T | T | T | G |
| | P18 | C | — | A | — | — | — | C | — | — | — | — | — | — | — |
| | P29 | C | — | A | — | — | A | C | — | — | — | — | — | — | — |
| | P35 | C | A | A | A | G | A | C | G | T | T | A | C | C | A |
| aa | WT | F | R | C | E | Q | D | N | K | — | — | L | Y | T | R |
| | P18 | S | — | Y | — | — | — | H | — | — | — | — | — | — | — |
| | P29 | S | — | Y | — | — | N | H | — | — | — | — | — | — | — |
| | P35 | S | K | Y | K | R | N | H | E | — | — | Q | H | — | K |

| | | | | | | | | | | | | |
|---|---|---|---|---|---|---|---|---|---|---|---|---|
| aa position | 129 | 129 | 231 | 294 | 307 | 475 | 488 | 35 | 113 | 133 | 177 | 403 |

| | | Nt pos | | | | | | | | | | | | |
|---|---|---|---|---|---|---|---|---|---|---|---|---|---|---|
| | | 8719 | 8720 | 8721 | 8772 | 8814 | 8854 | 9567 | 10768 | 11117 | 11139 | 11428 | 13099 | 13101 |
| Gene | | L | L | L | L | L | L | L | L | L | L | L | L | L |
| nt | WT | C | T | T | G | A | C | A | A | T | G | T | C | T |
| | P18 | A | C | G | A | G | T | G | — | C | A | — | — | — |
| | P29 | A | C | G | A | G | T | G | — | C | A | G | — | — |
| | P35 | A | C | G | A | G | T | G | C | C | A | G | A | C |
| aa | WT | L | L | L | K | P | L | K | M | V | E | W | P | P |
| | P18 | T | T | T | — | — | — | — | — | A | — | — | — | — |
| | P29 | T | T | T | — | — | — | — | — | A | — | G | — | — |
| | P35 | T | T | T | — | — | — | — | L | A | — | G | T | T |

| | | | | | | | | | | | | | |
|---|---|---|---|---|---|---|---|---|---|---|---|---|---|
| aa position | 537 | 537 | 537 | 554 | 568 | 582 | 819 | 1220 | 1336 | 1343 | 1440 | 1997 | 1997 |

Mutations in italics were found in a single codon

TABLE 6
Overview of mutations in NL/17/00 after cold-passaging

| | | Nt pos | | | | | | | | | |
|---|---|---|---|---|---|---|---|---|---|---|---|
| | | 27 | 4086 | 4458 | 4692 | 5078 | 6981 | 8534 | 10751 | 11339 | 11354 |
| | Gene | Le | F | F | GE-F | M2.1 | GE-G | L | L | L | L |
| nt | WT | G | A | G | A | C | A | T | T | G | G |
| | P14 | — | — | — | — | — | — | — | C | A | A |
| | P29 | — | — | A | del | A | — | A | C | A | A |
| | P35 | A | T | A | del | A | G | A | C | A | A |
| aa | WT | — | I | E | — | S | — | N | L | E | G |
| | P14 | — | — | — | — | — | — | — | — | — | — |
| | P29 | — | — | K | — | Y | — | K | — | — | — |
| | P35 | — | F | K | — | Y | — | K | — | — | — |

| | | | | | | | |
|---|---|---|---|---|---|---|---|
| aa position | 341 | 465 | 119 | 467 | 1206 | 1402 | 1407 |

TABLE 7
Overview of mutations in NL/1/00 after cold-passaging

| | | Nt pos | | | | | | | | | | | | | |
|---|---|---|---|---|---|---|---|---|---|---|---|---|---|---|---|
| | | 2568 | 3344 | 3364 | 3367 | 4468 | 4652 | 5401 | 5783 | 6296 | 7074 | 7922 | 9253 | 10598 | 13306 |
| | Gene | M | F | F | F | F | F | M2.2 | SH | G | G | L | L | L | Tr |
| nt | WT | T | A | C | T | G | A | G | C | A | C | T | A | G | A |
| | P14 | A | — | A | — | — | — | — | A | T | — | C | G | — | — |
| | P29 | — | T | A | C | — | — | — | A | T | — | C | G | T | G |
| | P35 | A | — | A | C | A | T | T | A | T | T | C | G | — | — |
| aa | WT | V | E | Q | S | E | G | G | I | D | T | L | K | M | — |
| | P14 | E | — | K | — | — | — | — | — | V | — | — | — | — | — |
| | P29 | E | V | K | P | — | — | — | — | V | — | — | — | I | — |
| | P35 | E | — | K | P | K | V | V | — | V | — | — | — | — | — |

| | | | | | | | | | | | | | |
|---|---|---|---|---|---|---|---|---|---|---|---|---|---|
| aa position | 130 | 93 | 100 | 101 | 468 | 529 | 45 | 90 | 10 | 270 | 736 | 689 | 1138 |

### 6.5 HMPV Growth in Different Cell Lines

mMPVs can be cultured in different cell lines in order to examine the characteristics of each virus. For example, the infectivity of different viruses can be characterized and distinguished on the basis of titer levels measured in culture. Alternatively, cells can be used to propagate or amplify strains of the virus in culture for further analysis.

In one example, tertiary monkey kidney cells were used to amplify hMPV. However, tertiary monkey kidney cells are derived from primary cells which may only be passaged a limited number of times and have been passaged three times in vivo. A number of monkey cell lines such as Vero, LLC-MK2, HEp-2, and lung fibroblast (LF1043) cells, were tested to test whether they could support hMPV virus replication (Table 8). Trypsin used was TPCK-trypsin (Worthington) at a concentration of 0.001 mg/ml. The growth of this virus in fertilized 10 day old chicken eggs was also tested. The infected eggs were incubated for 2 and 3 days at 37° C. prior to AF harvest. Virus titers were determined by plaque assay of infected cell lysates on Vero cells without trypsin, incubated for 10 days at 35° C., and immunostained using the guinea pig hMPV antiserum. The results showed that Vero cells and LLC-MK2 cells were the cell substrates most suitable for hMPV virus replication, resulting in virus stock titers of 10⁶-10⁷ pfu/ml. These titers were similar to those obtained from tMK cells. The addition of trypsin at a concentration of 0.01 mg/ml did not increase virus titers appreciably (Table 8).

TABLE 8
HMPV VIRUS GROWTH IN DIFFERENT CELL LINES

| | Trypsin used to | |
|---|---|---|
| Cell Substrate | grow virus | Virus titers on Vero cells (pfu/ml) |
| Vero | yes | 2.1 × 10⁷ |
| | no | 1.1 × 10⁷ |
| LLC-MK2 | yes | 2.3 × 10⁵ |
| Hep-2 | yes | cells died |
| LF 1043 (HEL) | yes | no virus recovered |
| | no | no virus recovered |
| tMK | yes | 1.0 × 10⁷ |
| eggs (10 days) | no | no virus recovered |

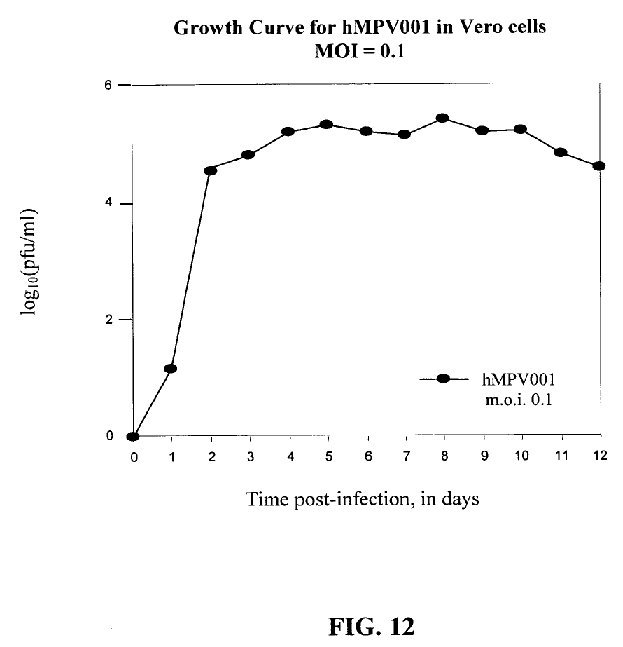

In order to study the virus kinetics of hMPV viral growth in Vero cells, a growth curve was performed using an MOI of 0.1 (FIG. 12). Cells and cell supernatants were harvested every 24 hours, and analyzed by plaque assay for quantification of virus titers. The results showed that at day 5, near peak titers of hMPV were observed. The absolute peak titer of 5.4 log₁₀ pfu/ml was achieved on Day 8. The virus titer was very stable up to day 10. A growth curve carried out at the same time with solely the cell supernatants, showed only very low virus titers. This data demonstrated that hMPV replication, under the conditions used (MOI of 0.1) peaked on day 8 post-infection and that hMPV was largely, a cell-associated RNA virus.

TRANSFECTION OF 293 CELLS: 293 cells (human kidney epithelial cells) were passed in DMEM and supplemented with FCS (10%), L-Glutamine (1:100) and Pen/Strep (1:100) and split 1:10 every 3-4 days. Care was taken not to let the cells grow to confluency in order to enhance transfectability. Cells were not very adherent; a very brief (2 min. or less) incubation in Trypsin-EDTA was usually sufficient to release them from plastic surfaces. Cells were diluted in culture media immediately after trypsin-treatment.

Cells were split the day before transfection. Cell confluency approximated 50-75% when transfected. Gelatinized plasticware was used to prevent cells from detaching throughout the transfection procedure. Plates or flasks were covered with 0.1% gelatinin (1:20 dilution of 2% stock) for 10 minuted and rinsed one time with PBS once. To achieve the correct cell density; cells were used at a concentration of 1×10⁷ cells per T75 flask or 100 mm plate (in 10 ml) or 1×10⁶ cells per well of a 6-well plate (in 2 ml).

Transfection lasted for a minimum of 7 hours, however, it was preferable to allow the transfection to occur overnight. The following were combined in a sterile tube: 30 mg DNA with 62 ml 2 M CaCl₂ and H₂O to 500 ml (T75) or 3 mg DNA with 6.2 ml 2 M CaCl₂ and H₂O to 50 ml (6-well plate); with brief mixing. Addition of 500 or 50 ml 2×HBS occurred dropwise and the solutions were allowed to mix for 5 minutes until a precipitate formed. Gentle care was used, i.e. no vortexing was applied. The old media was replaced with fresh prewarmed media (10 ml per T75 flask or 1 ml per well of a 6-well plate. The DNA was mixed carefully by blowing airbubles through the tube with a Gilson pipet and the precipitate was added dropwise to the media covering the cells. The cells were incubated in a 37° C. CO₂ atmosphere.

The cells appeared to be covered with little specks (the precipitate). The transfection media was removed from the cells, and the cells were rinsed carefully with PBS, and then replaced with fresh media. The cells were incubated in a 37° C. CO₂ atmosphere until needed, usually between 8-24 hours.

A 10× stock of HBS was prepared with 8.18% NaCl, 5.94% Hepes and 0.2% Na₂HPO₄ (all w/v). The solution was filter sterilized and stored at 4° C. A 2× solution was prepared by diluting the 10× stock with H₂O and adjusting the pH to 7.12 with 1 M NaOH. The solution was stored in aliquots at −20° C. Care was taken to exactly titrate the pH of the solution. The pH was adjusted immediately before the solution was used for the transfection procedure.

### 6.6 Minireplicon Construct of MPV

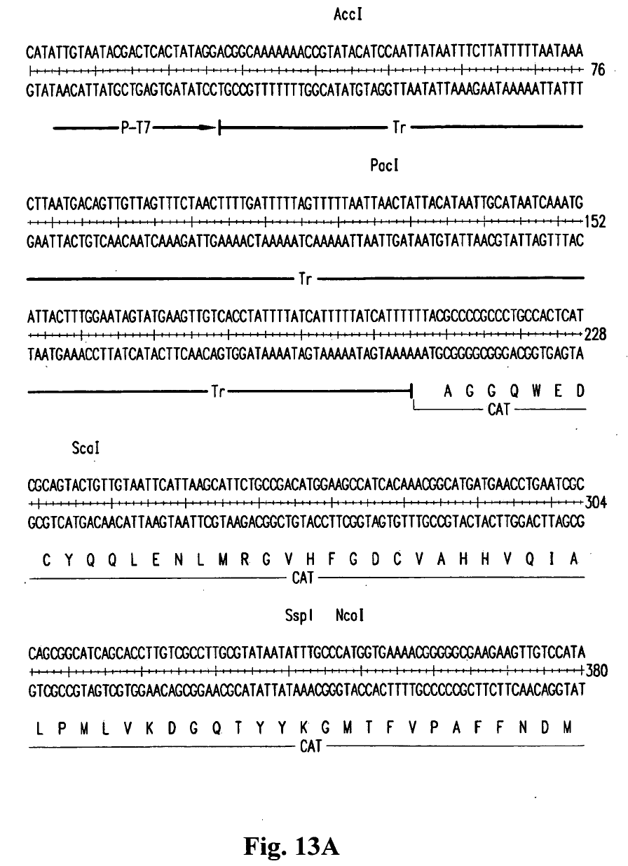

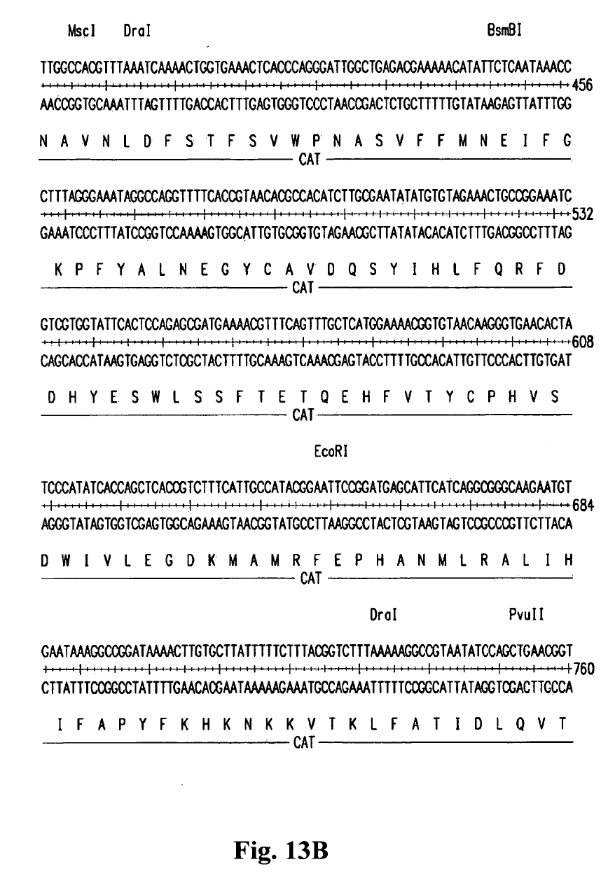

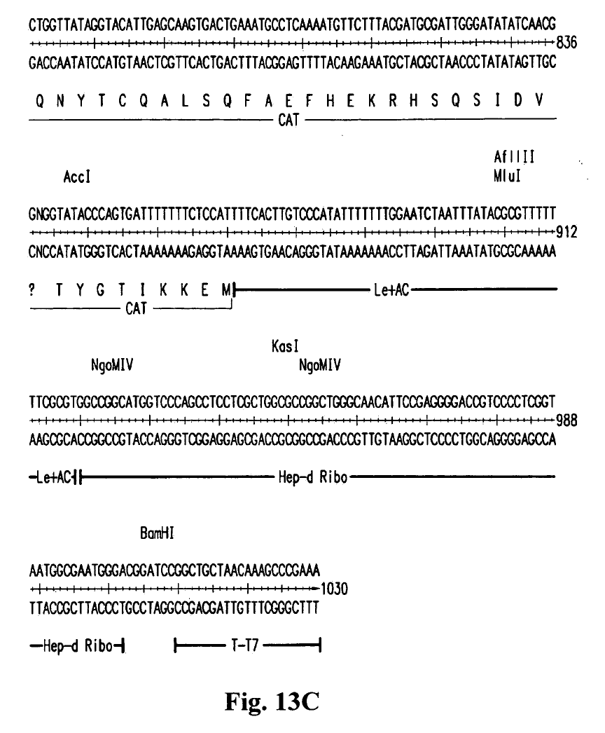

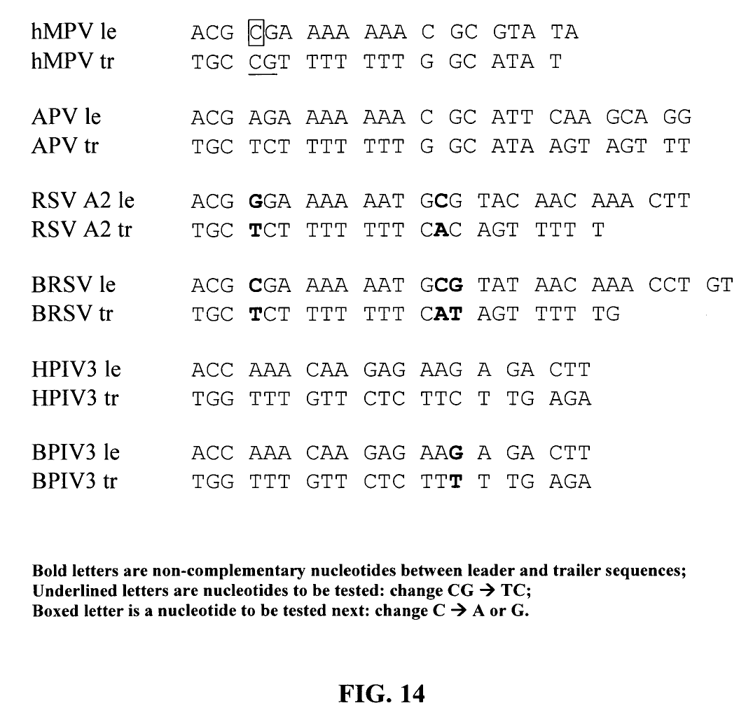

Minireplicon constructs can be generated to contain an antisense reporter gene. An example of a minireplicon, CAT-hMPV, is shown in FIG. 13. The leader and trailer sequences that were used for the generation of the minireplicon construct are shown in FIG. 14. For comparison, an alignment of APV, RSV and PIV3 leader and trailer sequences are also shown in FIG. 14.

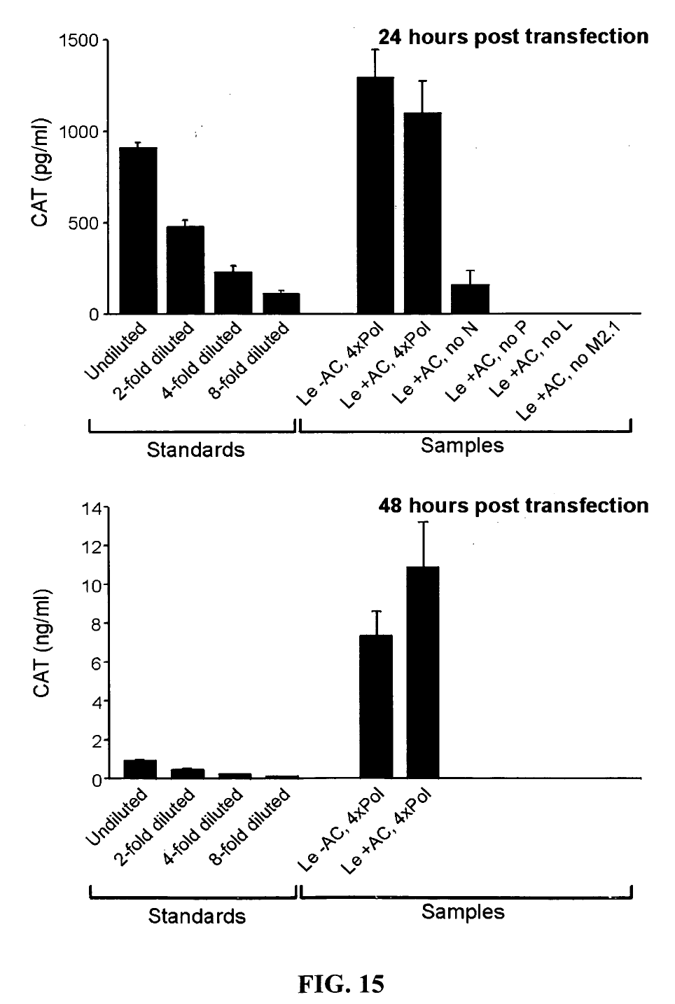

Two versions of the minireplicon constructs were tested: one with terminal AC residues at the leader end (Le+AC), and one without terminal AC residues at the leader end (Le−AC). The two constructs were both functional in the assay (FIG. 15). It can be seen in FIG. 15 that much higher CAT expression occurred after 48 hours than after 24 hours. After 48 hours, around 14 ng CAT per 500,000 cells transfected was observed. This experiment was entirely plasmid driven: the minireplicon was cotransfected with a T7 polymerase plasmid, and the N, P, L, M2.1 genes were expressed from pCITE-2a/3a (the pCite plasmids have a T7 promoter followed by the IRES element derived from the encephalomyocarditis virus (EMCV)). The CAT expression was completely abolished when L, P and N were excluded. A significant reduction in CAT expression was noted when M2.1 expression was excluded from the vector.

### 6.7 Rescue of HMPV From a Minireplicon Using RSV APV, MPV, or PIV Polymerase

In order to rescue hMPV, minireplicon constructs can be generated to contain a reporter gene. An example of a minireplicon, CAT-hMPV, is shown in FIG. 13. A cDNA encoding the reporter protein chloramphenicol acetyltransferase (CAT) can be cloned in negative-sense orientation between the 5′ and 3′ noncoding viral sequences. A T7 RNA polymerase promoter sequence and a recognition sequence for a restriction enzyme can flank the construct. In vitro transcription will yield virus-like RNA that will form reconstituted RNP complexes when mixed with purified polymerase proteins. The RNPs can be transfected into eucaryotic cells, for example, with helper virus. Alternatively, the rescue can be entirely plasmid driven, i.e., the minireplicon can be co-transfected with a T7 polymerase plasmid, and the N, P, L, and M2.1 genes expressed from pCITE-2a/3a. The polymerase components used to rescue hMPV can be those of RSV, APV, PIV, MPV, or any combination thereof (see above). Virus can be detected using any of a number of assays capable of detecting CAT activity. Rescue of hMPV using a minireplicon system can also be performed by superinfecting the minireplicon-transfected cells with hMPV polymerase components (NL/1/00 and NL/1/99) or polymerase components from MPV, APV, RSV, PIV, or any combination thereof.

A cDNA of the leader region and the adjoining gene can be modified by mutagenesis using synthetic oligonucleotides. Similarly, a cDNA of the downstream end of another hMPV gene, e.g., the L gene, and adjoining trailer region, can be modified to contain an adjacent T7 RNA polymerase promoter. The leader and trailer fragments can be cloned into an expression vector, e.g., pUC19, on either side of an insert of the CAT gene. cDNAs encoding additional hMPV viral analogs can be constructed in the same way. Construct structures can be confirmed using sequencing. Examples of the leader and trailer sequences that can be used for the generation of the minireplicon construct are shown in FIG. 14. For comparison, an alignment of APV, RSV and PIV3 leader and trailer sequences are also shown in FIG. 14.

### 6.8 Generation of Full Length Infectious cDNA

Full length cDNAs that express the genes of the hMPV virus can be constructed so that infectious viruses can be produced. For example, a cDNA encoding all of the genes or all of the essential genes of hMPV can be constructed; the genome can then be expressed to produce infectious viruses. Genetic alterations, such as mutations and non-native sequences, can be introduced into the cDNA by recombinant DNA technology.

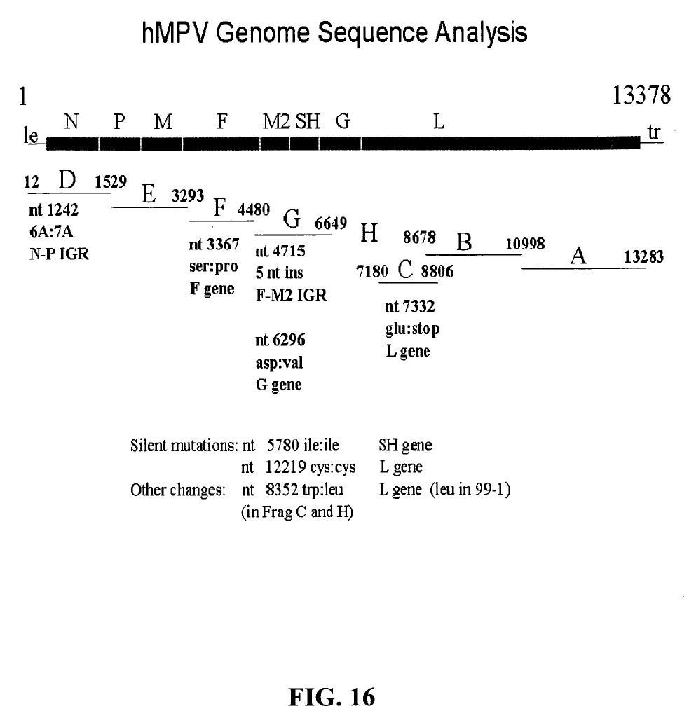

In order to genetically manipulate hMPV, the genome of this RNA virus was cloned. For the NL/1/00 isolate of hMPV, eight PCR fragments varying in length from 1-3 kb were generated (FIG. 16). The PCR fragments were sequenced and analyzed for sequence errors by comparison to the hMPV sequence deposited in Genbank. Two silent mutations (nucleotide 5780 ile:ile in the SH gene, nucleotide 12219 cys:cys in the L gene) were not corrected. Another change in the L gene at nucleotide 8352 (trp:leu) was not changed since this mutation was observed in two independently generated PCR fragments (C and H), as well as in the hMPV NL/1/99 sequence. Similarly, a 5 nucleotide insertion at nucleotide 4715 in the F-M2 intergenic region was not corrected. Both of these changes may be reflected in the wild type sequence of hMPV. In contrast, at nucleotide 1242, a single A residue was removed in the N—P intergenic region; at nucleotide 3367, a ser:pro was corrected in the F gene; at nucleotide 6296, an asp:val was changed in the G gene; and at nucleotide 7332 a stop codon was changed to a glu in the L gene.

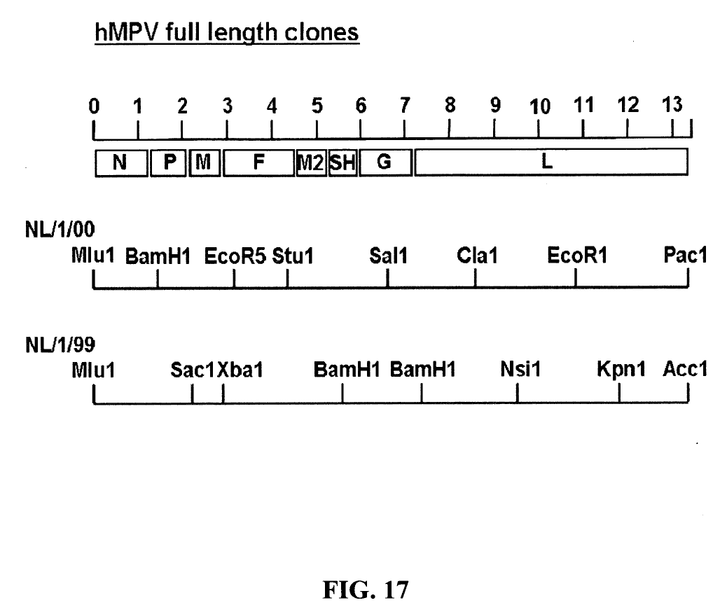

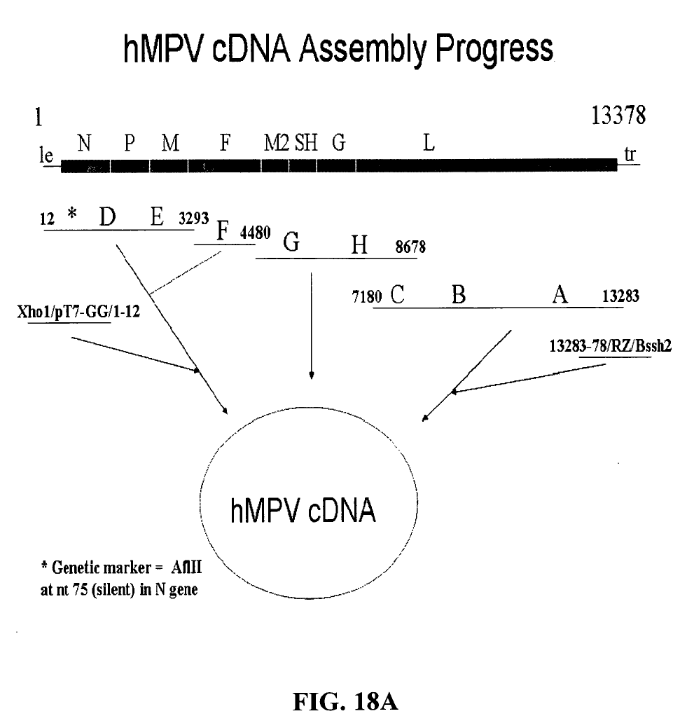

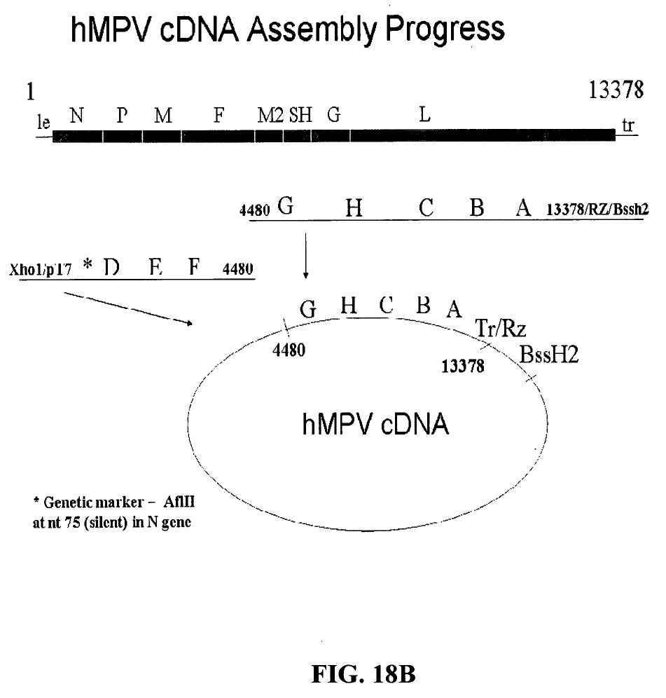

Restriction maps of different isolates of hMPV are shown in FIG. 17. The restriction sites can be used to assemble the full-length construct. The eight corrected PCR fragments were then assembled in sequence, taking advantage of unique restriction enzyme sites (FIG. 18). A genetic marker was introduced at nucleotide 75 generating an AflII restriction enzyme site without altering the amino acid sequence. A unique restriction enzyme site, XhoI, was added at the 3′ end of the hMPV sequence. A phage T7 polymerase promoter followed by two G residues was also added to the 3′ end of the hMPV sequence. At the 5′ end of the hMPV genome, a Hepatitis delta ribozyme sequence and BssHII restriction enzyme site were added. Helper plasmids encoding the hMPV L, N, P and M2.1 proteins in a pCITE plasmid were also generated. Once the full-length hMPV cDNA is generated, virus recovery by reverse genetics can be performed in Vero cells using fowl-pox T7 or MVA-T7 as a source of T7 RNA polymerase, or a cell line or a plasmid expressing T7 RNA polymerase.

### 6.9 HMPV Recovery Employing the Pol I—Pol II Promoter System

Unlike the reverse genetics systems for non segmented RNA viruses which are based on plasmids with T7-promoter for expression of genomic RNA, systems employing the cellular transcription machinery may be more efficient and do not require the coexpression of the RNA polymerase derived from the bacteriophage T7. A unidirectional or bi-directional pol I-pol II transcription system can be used to express viral RNA molecule intracellularly. This systems proved to be very efficient for the generation of influenza virus from cloned cDNA (Hoffmann et. al., PNAS, 97 6108-6113 (2000). Unlike RNA polymerase II transcripts, RNA polymerase I transcripts do not contain cap structures at their 5′-end and do not have poly A tails at the 3′-end. Thus, systems employing the cellular transcription machinery are designed to express proteins from a pol II promoter and viral (−)vRNA or (+) cRNA which do not have a cap structure or a polyA tail from a pol I promoter. To provide virus-like prirnary transcripts which do not contain additional non viral sequences is critical because the terminal structures are crucial for viral replication and transcription.

In order to evaluate whether (−)vRNA or (+)cRNA transcription of hMPV cDNAs by RNA polymerase I is more efficient, a minigenome system may be designed to compare the replication efficiency of each. Replication efficiency can be measured by the transcription of a reporter molecule expressed by the minigenome, e.g., a CAT gene. In this approach, plasmids expressing the L, N, P, and M2.1 genes of hMPV, under the control of a pol II promoter, are cotransfected into a host cell together with a CAT-minigenome-plasmid. The relative efficiency of replication is measured by determining the relative level of expression of the CAT reporter molecule.

For example, RNA pol I can be used to synthesize positive strand copies of the hMPV viral genome (cRNA). In brief, the viral cDNA is inserted between an RNA pol I promoter and a terminator sequence. The whole pol I transcription unit is inserted in the positive-sense orientation between an RNA pol II promoter and a polyadenylation site. Two types of positive-sense RNAs are synthesized. From the pol II promoter, an mRNA with a 5′-cap structure is transcribed. From the pol I promoter full-length, positive-sense hMPV cRNA with a triphosphate group at the 5′ end is transcribed by cellular RNA polymerase I. A cloning vector can be used for the insertion of arbitrary cDNA fragments, e.g., pHW11 (Hoffmann & Webster, J. Gen Virol. 2000 December 81(Pt 12):2843-7). This plasmid contains the pol II promoter (immediate early promoter of the human cytomegalovirus) and the human pol I promoter that are upstream of a pol I terminator sequence and a poly(A) site. In order to replicate the primary transcript representing viral cRNA, the viral polymerase proteins are provided by plasmid vectors with a pol II promoter, such as the immediate early promoter of human cytomegalovirus. These plasmids contain the cDNAs representing four gene segments of hMPV, i.e., the L-gene, the N-gene, the P-gene, and the M2.1 gene. Those four plasmids (1-5 μg) are cotransfected with the pol I/pol II plasmid (1-5 μg) representing the full length genome of hMPV into 10⁶-10⁷ 293T cells, COS-7 or Vero cells. To improve the efficiency and reliability of the system, 293T cells can be cocultured with cells permissive for MPV, such as Vero or tMK cells. The addition of trypsin to the cell culture medium results in the generation of infectious virus particles. The coculturing of primate cells with MDCK cells was employed for the efficient rescue of influenza A virus (Hoffmann et. al., PNAS, 97 6108-6113 (2000). The supernatant after different times after transfection (i.e., 3d to 10d) is titrated and transferred to fresh Vero cells to determine the virus titer. The coexpression of all viral structural proteins (i.e., M, M2.2, SH, F, and G) from a pol II promoter may improve the efficiency of virus recovery.

Because from the pol I/pol II plasmids with the full length cDNA a capped and non-capped RNA is produced, it is expected that the first open reading frame representing the N-gene is translated into N-protein. Thus, by employing the pol I/pol II approach only four plasmids are needed for virus rescue: Three pol I-plasmids expressing L, P, and M2.1 protein and one pol I/pol II plasmid expressing the N-protein and the full length RNA of MPV.

### 6.10 Rescue of HMPV

A successful system was developed to rescue recombinant hMPV. In brief, expression plasmids, encoding various polymerase proteins, were co-transfected with the cloned hMPV to be rescued into appropriate host cells. Upon collection and treatment, the cells and supernatant were then used to inoculate Vero cells. Infectious rescued virus was detected using immunostaining methods. In order to rescue hMPV, confluent monolayers of 293T cells in a TC6-well plate were inoculated with fowl pox virus at a MOI (multiplicity of infection)=0.5. The cells were then incubated at 35° C. for 1 hour. The expression plasmids and the cloned hMPV to be rescued were mixed in 100 μl optiMEM (per well) in the following amounts: 0.4 μg of plasmid encoding the hMPV P gene (in pCITE 2a/3a, designated clone #41-6), 0.4 μg of plasmid encoding the hMPV N gene (in pCITE 2a/3a, designated clone #35-11), 0.3 μg of plasmid encoding the hMPV M2 gene (in pCITE 2a/3a, designated clone #25-6), 0.2 μg of plasmid encoding the L gene (in pCITE 2a/3a, designated clone #2), and 4 μg of hMPV plasmid clone #2 which has the leader and trailer like APV or clone #10 which has hMPV leader and trailer sequences. It is noteworthy that the expression plasmids used have the wild type sequence restored in the second amino acid position.

In the next step, the transfection reagent Lipofectamine 2000 (8 μl) was mixed into 100 μl of optiMEM and then added to the plasmid mixture. This combined mixture was applied to the 293T cells. Six days after transfection, the cells and supernatant were collected, frozen, thawed, and used to inoculate Vero cells. Nine days post inoculation, the infected cells were fixed in methanol, immunostained with a guinea pig polyclonal antibody followed by anti-guinea pig HRP and the DAKO AEC substrate. Plaque formation demonstrated that the rescued virus was infectious. Positive red immunostaining was evident in the wells with both clone #2 and #10, though more immunostained cells were in the well with hMPV clone #2 which has the APV leader and trailer compared to the clone #10 with the hMPV leader and trailer. These results indicate that recombinant hMPV was successfully rescued and that infectious virus was produced.

### 6.11 Infection of Animal Hosts with Subtypes of HMPV

Animal hosts can be infected in order to characterize the virulence of MPV strains. For example, different hosts can be used in order to determine how infectious each strain is in an organism. Balb/c mice, cotton rats, and Syrian Golden hamsters were infected with hMPV using a dose of 1.3×10⁶ pfu/animal. The animals were inoculated intranasally with 1.3×10⁶ pfu of hMPV in a 0.1 ml volume. The tissue samples were quantified by plaque assays that were immunostained on Day 9 with the hMPV guinea pig antiserum. Four days post-infection, the animals were sacrificed, and the nasal turbinates and lungs were isolated and quantified for hMPV titers by plaque assays that were immunostained (Table 9).

TABLE 9
HMPV TITERS IN INFECTED ANIMALS

| | | Mean virus titer on day 4 | |
|---|---|---|---|
| | | post-infection (log₁₀ PFU/g | |
| | Number of | tissue +/− Standard Error | |
| Animals | animals | Nasal turbinates | Lungs |
| mice (Balb c) | 6 | 2.7 +/− 0.4 | 2.2 +/− 0.6 |
| cotton rats | 5 | <1.7 +/− 0.0 | <1.8 +/− 0.0 |
| Syrian Golden | 6 | 5.3 +/− 0.2 | 2.3 +/− 0.6 |
| hamsters | | | |

The results showed that hMPV replicated to high titers in Syrian Golden hamsters. Titers of 5.3 and 2.3 log10 pfu/g tissue were obtained in the nasal turbinates and lungs, respectively. hMPV did not replicate to any appreciable titer levels in the respiratory tracts of cotton rats. Mice showed titers of 2.7 and 2.2 log₁₀ pfu/g tissue in the upper and lower respiratory tracts, respectively. These results suggested that Syrian Golden hamsters would be a suitable small animal model to study hMPV replication and immunogenicity as well as to evaluate hMPV vaccine candidates.

INFECTION OF GUINEA PIGS. Two virus isolates, NL/1/00 (subtype A) and NL/1/99 (subtype B), were used to inoculate six guinea pigs per subtype (intratracheal, nose and eyes). Six guinea pigs were infected with hMPV NL/1/00 (10e6,5 TCID50). Six guinea pigs were infected with hMPV NL/1/99 (10e4,1 TCID50). The primary infection was allowed to progress for fifty-four days when the guinea pigs were inoculated with the homologous and heterologous subtypes (10e4 TCID50/ml), i.e., two guinea pigs had a primary infection with NL/1/00 and a secondary infection with NL/1/99 in order to achieve a heterologous infection, three guinea pigs had a primary infection with NL/1/00 and a secondary infection with NL/1/00 to achieve a homologous infection, two guinea pigs had a primary infection with NL/1/99 and a secondary infection with NL/1/00 to achieve a heterologous infection and three guinea pigs had a primary infection with NL/1/99 and a secondary infection with NL/1/99 to achieve a homologous infection.

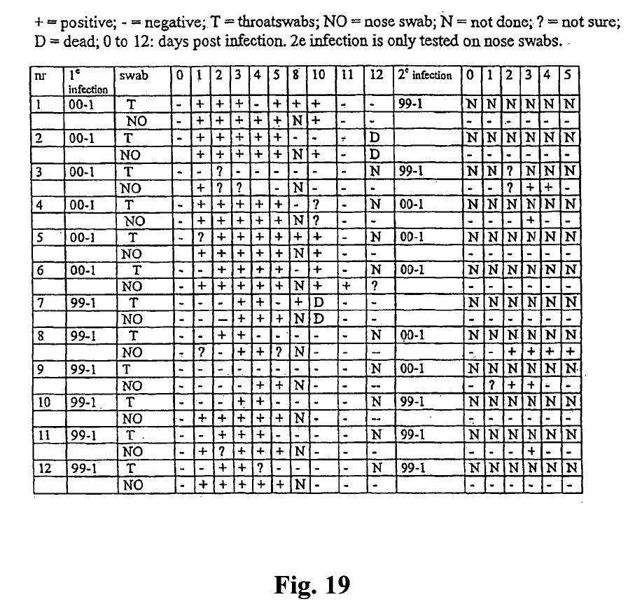

Throat and nose swabs were collected for 12 days (primary infection) or 8 days (secondary infection) post infection, and were tested for the presence of the virus by RT-PCR assays. The results (FIG. 19) of the RT-PCR assays showed that guinea pigs inoculated with virus isolate NL/1/00 showed infection of the upper respiratory tract on days 1 through 10 post infection. Guinea pigs inoculated with 99-1 showed infection of the upper respiratory tract day 1 to 5 post infection. Infection of guinea pigs with NL/1/99 appeared to be less severe than infection with NL/1/00. A second inoculation of the guinea pigs with the heterologous virus, as commented on above, resulted in re-infection in 3 out of 4 of the guinea pigs. Likewise, reinfection in the case of the homologous virus occurred in 2 out of 6 guinea pigs. Little or no clinical symptoms were noted in those animals that became re-infected, and no clinical symptoms were seen in those animals that were protected against the re-infections, demonstrating that even with the wild-type virus, a protective effect due to the first infection may have occurred. This also showed that heterologous and homologous isolates could be used as a vaccine.

Both subtypes of hMPV were able to infect guinea pigs, although infection with subtype B (NL/1/99) seemed less severe, i.e., the presence of the virus in nose and throat was for a shorter period than infection with subtype A (NL/1/00). This may have been due to the higher dose given for subtype A, or to the lower virulence of subtype B. Although the presence of pre-existing immunity did not completely protect against re-infection with both the homologous and heterologous virus, the infection appeared to be less prominent, in that a shorter period of presence of virus was noted and not all animals became virus positive.

INFECTION OF CYNOMOLOGOUS MACAGUES. Virus isolates NL/1/00 (subtype A) and NL/1/99 (subtype B) (1e5 TCID50) was used to inoculate two cynomologous macaques per subtype (intratracheal, nose and eyes). Six months after the primary infection, the macaques were inoculated for the second time with NL/1/00. Throat swabs were collected for 14 days (primary infection) or 8 days (secondary infection) post infection, and were tested for presence of the virus by RT-PCR assays.

Cynomologous macaques inoculated with virus isolate NL/1/00 showed infection of the upper respiratory tract day 1 to 10 post infection. Clinical symptoms included a suppurative rhinitis. A second inoculation of the macaques with the homologous virus results in re-infection, as demonstrated by PCR, however, no clinical symptoms were seen.

Sera were collected from the macaques that received NL/1/00 during six months after the primary infection (re-infection occurred at day 240 for monkey 3 and day 239 for monkey 6). Sera were used to test for the presence of IgG antibodies against either NL/1/00 or APV, and for the presence of IgA and IgM antibodies against 00-1.

Two macaques were succesfully infected with 00-1 and in the presence of antibodies against NL/1/00 were reinfected with the homologous virus. The response to IgA and IgM antibodies showed the raise in IgM antibodies after the first infection, and the absence of it after the reinfection. IgA antibodies were only detected after the re-infection, showing the immediacy of the immune response after a first infection. Sera raised against hMPV in macaques that were tested in an APV inhibition ELISA showed a similar response as to the hMPV IgG ELISA.

Antibodies to hMPV in cynomologous macaques were detected with the APV inhibition ELISA using a similar sensitivity as that with the hMPV ELISA, and therefore the APV inhibition EIA was suitable for testing human samples for the presence of hMPV antibodies.

Virus cross-neutralization assays were preformed on sera collected from hMPV infected cynomologous macaques. The sera were taken from day 0 to day 229 post primary infection and showed only low virus neutralization titers against NL/1/00 (0-80), the sera taken after the secondary infection showed high neutralisation titers against NL/1/00, i.e., greater than 1280. Only sera taken after the secondary infection showed neutralization titers against 99-1 (80-640), and none of the sera were able to neutralize the APV C virus. There was no cross reaction between APV-C and hMPV in virus cross-neutralization assays, however, there was a cross reaction between NL/1/00 and NL/1/99 after a boost of the antibody response.

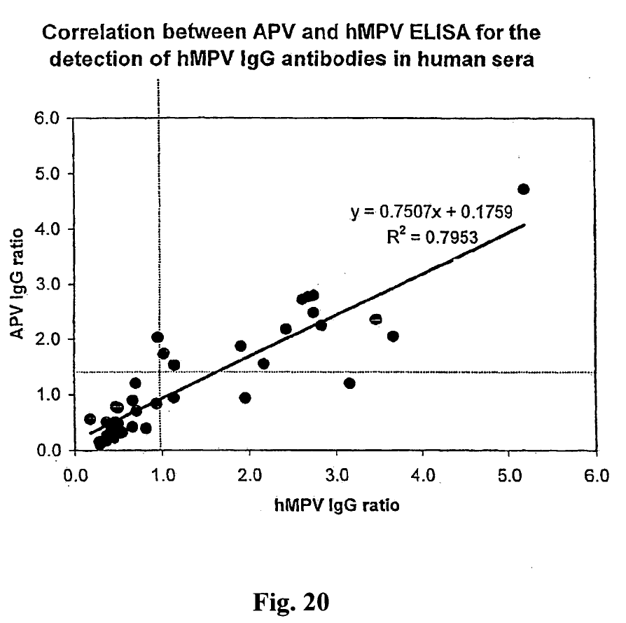

INFECTION OF HUMANS. The sera of patients ranging in ages under six months or greater than twenty years of age were previously tested using IFA and virus neutralization assays against 00-1. These sera were tested for the presence of IgG, IgM and IgA antibodies in an ELISA against NL/1/00. The samples were also tested for their ability to in inhibit the APV ELISA. A comparison of the use of the hMPV ELISA and the APV inhibition ELISA for the detection of IgG antibodies in human sera was made and a strong correlation between the IgG hMPV test and the APV-Ab test was noted, therefore the APV-Ab test was essentially able to detect IgG antibodies to hMPV in humans (FIG. 20).

INFECTION OF POULTRY. The APV inhibition ELISA and the NL/1/00 ELISA were used to test chickens for the presence of IgG antibodies against APV. Both the hMPV ELISA and the APV inhibition ELISA detected antibodies against APV.

### 6.12 M2 Deletion Mutants

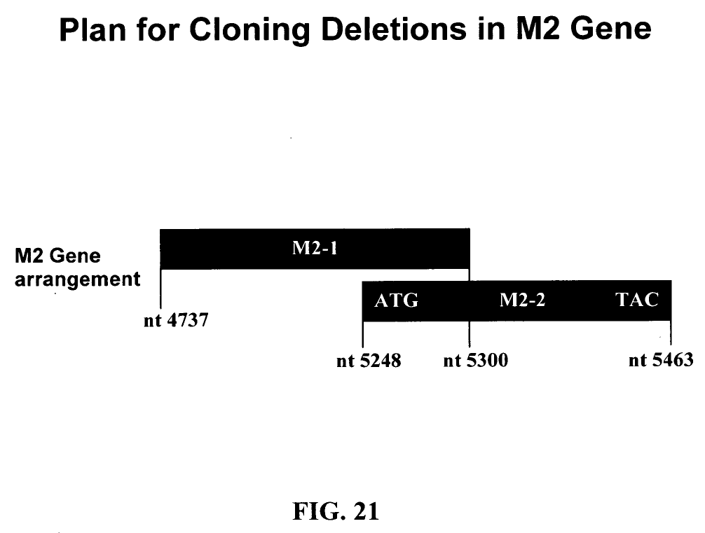

A map of the M2 gene of hMPV strain hMPV/NL/1/00 is shown in FIG. 21. In order to generate a deletion of the M2 gene, a Bsp E1 site is constructed at nucleotide position 4741 and a second Bsp E1 site is constructed at nucleotide position 5444. The restriction sites are constructed by site-specific mutagenesis. Restriction digestion of the recombinant genome at the two Bsp E1 sites using the restriction endonuclease Bsp E1 and subsequent ligation results in a deletion of the sequence between nucleotide position 4741 and nucleotide position 5444.

In order to generate a deletion of the M2.1 open reading frame of the M2 gene, Nhe I sites are introduced at nucleotide positions 4744 and 5241. The restriction sites are constructed by site-specific mutagenesis. Restriction digestion of the recombinant genome at the two Nhe I sites using the restriction endonuclease Nhe I and subsequent ligation results in a deletion of the sequence between nucleotide position 4744 and nucleotide position 5241.

In order to generate a deletion of the M2.2 open reading frame of the M2 gene, Swa I sites are introduced at nucleotide positions 5311 and 5453. The restriction sites are constructed by site-specific mutagenesis. Restriction digestion of the recombinant genome at the two Swa I sites using the restriction endonuclease Swa I and subsequent ligation results in a deletion of the sequence between nucleotide position 5311 and nucleotide position 5453.

The following primer sets were used: primers used to introduce the restriction enzyme sites:

For putting BspEI into hMPV/NL/1/00 to make M2 deletion from 4741 to 5444:

### Primer Set

(SEQ ID NO:105)

| | |
|---|---|
| “hMPV, BspEI, +4741” | gga caa atc ata acg t tcc |
| | gga ag gc tcc gtg c |

(SEQ ID NO:106)

| | |
|---|---|
| “hMPV, BspEI, −4741” | g cac gga gc ct tcc gga |
| | acgt tat gat ttg tcc |
| and primer set | |

(SEQ ID NO:107)

| | |
|---|---|
| “hMPV, BspEI, +5444” | cat agaaat tat at atg tcc |
| | gga ct ta ctt a agt tag |

(SEQ ID NO:108)

| | |
|---|---|
| “hMPV, BspEI, −5444” | cta act t aa g ta ag tcc |
| | gga cat at ata att tc |

For putting Nhe I sites into hMPV to make M2.1 deletion from 4744 to 5241 and change the start site from atg to acg at nt 4742:

### Primer Set

(SEQ ID NO:109)

| | |
|---|---|
| “hMPV, Nhe I, +4744” | gga caa atc ata ac g g ct |
| | agc aag gc t ccg tgc |

(SEQ ID NO:110)

| | |
|---|---|
| “hMPV, NheI, −4744” | gca cgg agc ctt gct agc cgt |
| | tat gat ttg tcc |
| primer set | |

(SEQ ID NO:111)

| | |
|---|---|
| “hMPV, NheI, +5241” | ctt atc agc agg t gctagc a |
| | atg act ctt cat a tg c |

(SEQ ID NO:112)

| | |
|---|---|
| “hMPV, Nhe I, −5241” | gcat atg aa g ag t ca t t |
| | gct a gc a cct gct gat aag |

For putting Swal sites into hMPV to make M2.2 deletion from 5311 to 5453:

### Primer Set

(SEQ ID NO:113)

| | |
|---|---|
| “hMPV, SwaI, +5311” | c agt gag cat ggt cca att |
| | taa att act ata gag g |

(SEQ ID NO:114)

| | |
|---|---|
| “hMPV, SwaI, −5311” | c ctc tat agt aat tta aat |
| | tgg acc atg ctc act g |
| and primers | |

(SEQ ID NO:115)

| | |
|---|---|
| “hMPV, SwaI, +5453” | c ata gaa att ata tat gtc |
| | aag gct tat tta aat tag |

(SEQ ID NO:116)

| | |
|---|---|
| “hMPV, SwaI, −5453” | cta att taa ata agc ctt gac |
| | ata tat aat ttc tat g |

For the generation of hMPV (strain hMPV/NL/1/00) with a deletion in SH, cloned with deletion from 5472 to 6026, has been recovered and grows well in Vero cell culture. The primer sets for cloning the hMPV/NL/1/00 virus with the SH deletion are as follows:

### Primer Set

(SEQ ID NO:117)

| | |
|---|---|
| hMPV SacII +5472 | ggc tta ctt aag tta gta aaa |
| | aca ccg cgg agt ggg ata aat gac |

(SEQ ID NO:118)

| | |
|---|---|
| hMPV SacII −5472 | gtc att tat ccc act ccg cgg |
| | tgt ttt tac taa ctt aag taa |
| | gcc |
| primer set | |

(SEQ ID NO:119)

| | |
|---|---|
| hMPV SacII +6026 | ct atc att acc caa ccgcgg |
| | aa acc caa tcc taa atg tta |
| | ac |

(SEQ ID NO:120)

| | |
|---|---|
| r hMPV SacII −6026 | gt taa cat tta gga ttg ggt |
| | tt ccgcgg ttg ggt aat gat |
| | ag |

### 6.13 Plasmid-Only Recovery of hMPV in Serum Free Vero Cells by Electroporation

(a) Introduction

This process allows recovery of recombinant hMPV using plasmids only, in the absence of helper viruses. The recovery of hMPV is carried out using SF Vero cells, which are propagated in the absence of animal and human derived products. This process allows recovery of recombinant hMPV with similar efficiency to previous methods using helper viruses (recombinant vaccinia or fowl-pox viruses expressing T7 polymerase). Because no helper viruses are needed in the recovery process, the vaccine viruses are free of contaminating agents, simplifying downstream vaccine production. The cells used for vaccine virus recovery are grown in media containing no animal or human derived products. This eliminates concerns about transmissible spongiform encephalopathies (e.g. BSE), for product end users.

This method enables generation of a recombinant vaccine seed that is completely free of animal or human derived components. The seed is also free of contaminating helper viruses.

Plasmid-based expression systems for rescue of viruses from cDNA are described, e.g., in R A Lerch et al., Wyeth Vaccines, Pearl River N.Y., USA (Abstract 206 from XII International Conference on Negative Strand Viruses, Jun. 14-19, 2003, Pisa Italy) and G. Neumann et. al., J. Virol., 76, pp 406-410.

(b) Methods and Results

hMPV N plasmids (4 μg; marker: kanamycin resistancy), hMPV P plasmids (4 μg; marker: kanamycin resistancy), hMPV L plasmids (2 μg; marker: kanamycin resistancy), cDNA encoding hMPV antigenomic cDNA (5 μg; marker: kanamycin resistancy) and pADT7(N)DpT7 encoding T7 RNA polymerase (5 μg; marker: blasticidin) are introduced into SF Vero cells using electroporation in serum-free medium.

For the rescue of hMPV virus, 4 expression plasmids are used. They are for the genes N, P, L and also M2 of hMPV. In particular the following plasmids are used:

4 ug hMPV N pCITE plasmid,

4 ug hMPV P pCITE plasmid,

3 ug hMPV M2 pCITE plasmid,

2 ug hMPV L pCITE plasmid

5 ug T7 RNA polymerase plasmid,

and 5 ug of the viral cDNA encoding the viral genome to be be rescued.

The pCITE plasmid has an internal ribosomal entry site that functions in the cytoplasm of the Vero cell so that the proteins for the N, P, M2 and L are made in the cytoplasm. These proteins form the viral polymerase complex.

The viral genome to be rescued is in a full length plasmid with a T7 promoter. Without being bound by theory, T7 DNA-dependent RNA polymerase transcribes a full length viral RNA genome using this full length plasmid. After the viral genome is made, the viral polymerase complex will transcribe the viral genome and generate viral messenger RNAs and virus is subsequently recovered.

The pulse for the electroporation is 220V and 950 microfarads. 5.5×106 SF Vero cells are used per electroporation. The electroporated cells are allowed to recover at 33° C. in the presence of OptiC (a custom formulation from GIBCO Invitrogen Corporation) overnight. Recovered cells are washed twice with 1 mL of PBS containing calcium and magnesium and overlayed with 2 mL of OptiC. Electroporated cells are further incubated at 33° C. for 5-7 days. At the end of the incubation period, cells are scraped into the media and total cell lysate is analyzed for presence of hMPV. Virus recovery is confirmed by immunostaining of plaque assays using hMPV specific polyclonal antibodies.

### 6.14 Growth Behavior of Recombinant hMPV

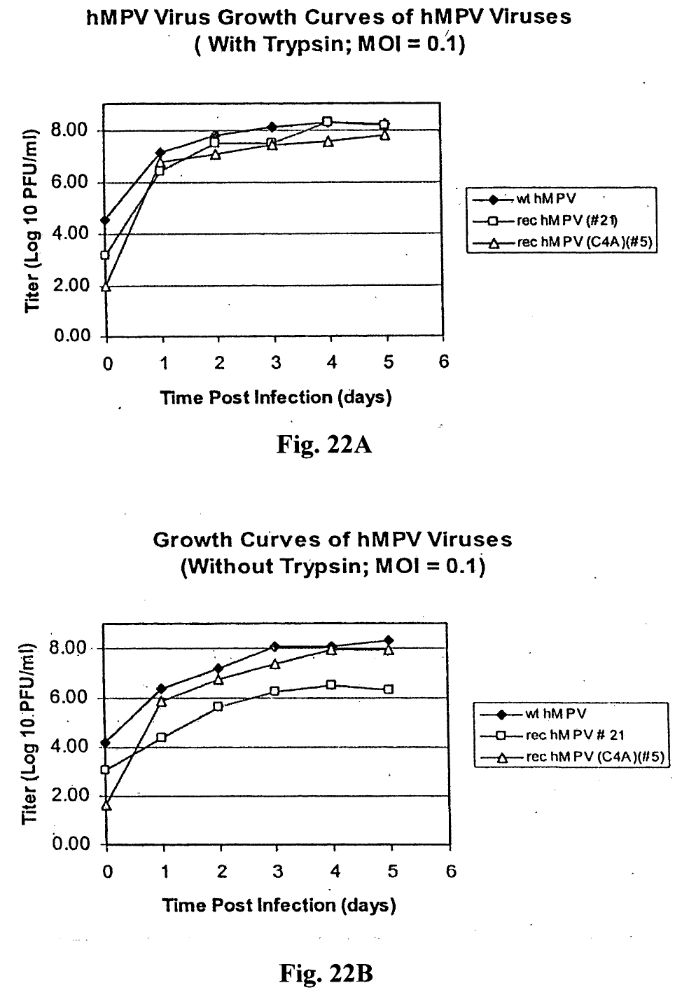

Several recombinant hMPV were constructed and rescued as described above. Growth curves of recombinant hMPVJNL/1/00 in the presence and absence of Trypsin are shown in FIG. 22. The cells (Vero cells) were infected at a MOI of 0.1.

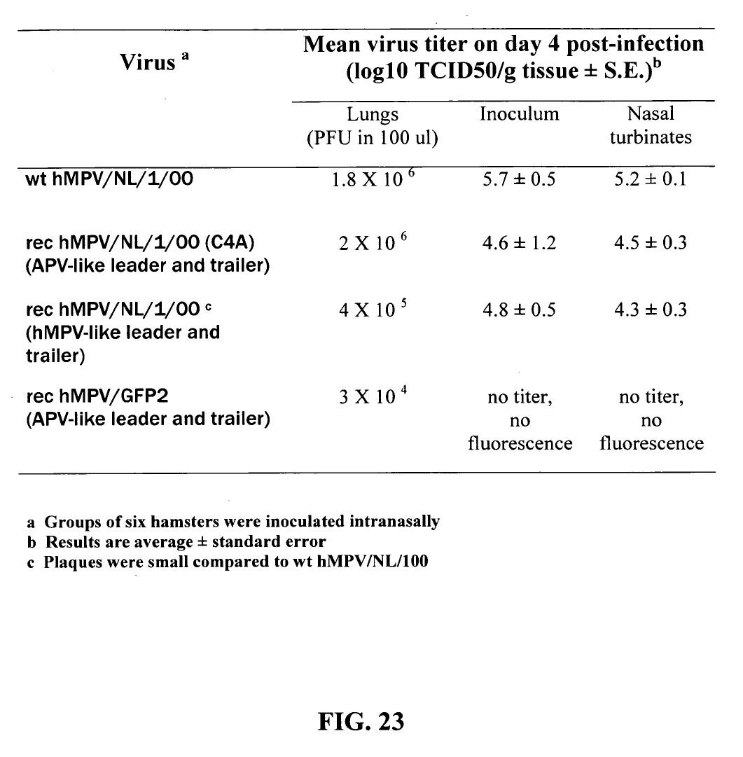

Replication of wild type hMPVINL/1/00 and recombinant hMPV/NL/1/00 in the upper and lower respiratory tract of hamsters are shown in FIG. 23. Hamsters were infected as described above.

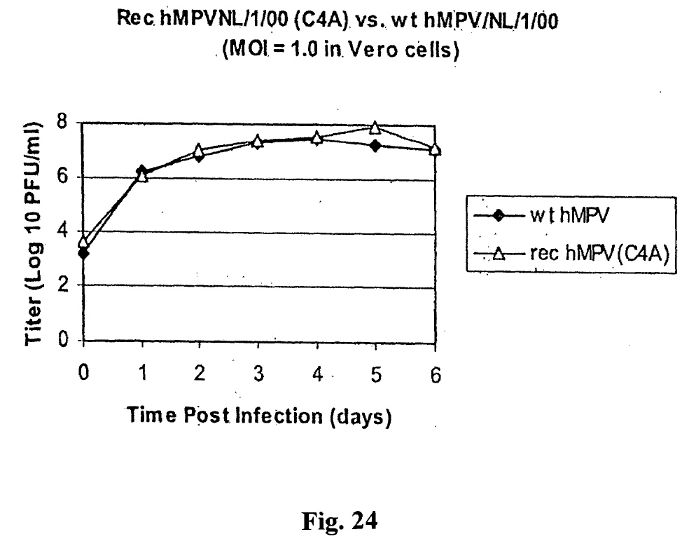

A growth curve of a recombinant hMPV/NL/1/00 with a cytosine to adenine at position 4 of the leader sequence (“C4A”) compared to wild-type hMPV/NL/1/00 is shown in FIG. 24. The cells (Vero cells) were infected at a MOI of 1.

### 6.15 Microneutralization Assay Using hMPV/GFP2

When viruses are inoculated into an animal, an array of antibodies against the virus are produced. Some of these antibodies can bind virus particles and neutralize the infectivity of the viruses. In this example, a microneutralization assay was used to analyze the remaining infectivity of the viruses after the viruses were incubated with dilutions of serum containing antibodies. For serial dilutions, a 96-well plate is divided (i) into rows A (dilution 1:32); B (1:64); C (1:128); D (1:256); E (1:512); F (1:1024); G (1:2048); and H (No Antibody) and (ii) into columns 1 to 12 for the different samples (first sample: columns 1 to 3; second sample: columns 4 to 6; third sample: columns 7 to 9; and fourth sample: columns 10 to 12). 230 PI of sample dilution are added to row A. 115 μl of Opti-MEM are added to rows B-H. Then 115 μl of the 1:32 dilution of the first sample are added to wells 1B, 2B, and 3B, the second sample to wells 4B, 5B, and 6B, the third sample to wells 7B, 8B, and 9B, and the fourth sample to 10B, 11B, and 12B. Sera and medium are mixed gently by pipetting up and down three times. The steps are repeated for rows B to C, rows C to D, rows D to E, rows E to G. After diluted sample is added to row G and mixed, 115 μl are removed from row G and discarded.

Microneutralization assay was performed as follows: sera were serially diluted. Each test sample and each control was diluted by 1:32 by adding 22.5 PI of sera to 697.5 μl of Opti-MEM Medium (1×). Serum and medium were mixed gently by inversion three times and place on ice. Each dilution of serum was incubated with the virus hMPV/GFP2. Cells were washed with phosphate buffered saline (“PBS”). Vero cells from ATCC are maintained in MEM (JRH Biosciences) supplemented with 10% fetal bovine serum, 2 mM L-glutamine, nonessential amino acids, and 100 units/ml penicillin G, 100 μg/ml streptomycin sulfate. The virus/sera mixtures were added to cells and incubated for one hour at 35° C. All of the medium, which contained the virus, were removed, and cells were washed with PBS. Opti-MEM medium was added to the cells and the cell cultures were incubated for three days. Opti-MEM I Reduced-Serum Medium (1×) (GIBCO 31985-070) contains, among others, HEPES buffer, 2400 mg/L sodium bicarbonate, hypoxanthine, thymidine, sodium pyruvate, L-glutamine, trace elements, growth factors, and phenol red reduced to 1.1 mg/L. The remaining infectivity of the viruses was measured by quantifying eGFP green foci on the images captured with fluorescence microscope. Plaque reduction assay using a wildtype virus, e.g., wildtype hMPV/NL/1/00, was also performed for comparing the sensitivity of the microneutralization assay. The results are presented in Tables 10 to 12.

The results demonstrate that the microneutralization assay using hMPV/GFP2 provides reliable and reproducible results. The use of hMPV-GFP in the microneutralization assay facilitates the high throughput screening of different vaccines and antibodies in animal model systems such as ferrets and monkeys. This technique also provides efficient means for diagnosing and monitoring infections in humans. These results demonstrate a linear correlation between plaque reduction and microneutralization using hMPV/GFP2.

TABLE 10
Titers of ferret sera using hMPV/GFP2 microneutralization
assay and plaque reduction assay.

| | Plaque (NT50) | Microneutralization (NT50) | |
|---|---|---|---|
| | −trypsin | −trypsin | +trypsin |
| | +complement | −complement | −complement |
| Ferret sera | Wildtype hMPV/NL/1/00 | hMPV/GFP2 | hMPV/GFP2 |
| 1 | 5.9 | 8.2 | 8.3 |
| 2 | 3.3 | 8.7 | 6.9 |
| 3 | 4.1 | 6.8 | 7.1 |
| 4 | 3.6 | 8.5 | 5.1 |
| 5 | 2.9 | 6.0 | 7.9 |

Complement from Guinea pig (add 100 μl in 20 ml of Opti-MEM) was used for plaque reduction assay. NT50 is 1/dilution that confers 50% neutralization of input virus. The numbers in the table indicate the titers of sera.

TABLE 11
Titers of Monkey sera using hMPV/GFP2 microneutralization
assay and plaque reduction assay.

| | | Microneutralization |
|---|---|---|
| | Plaque (NT50) | (NT 50) |
| | −trypsin | +trypsin |
| | +complement | −complement |
| Monkey sera | Wildtype hMPV/NL/1/00 | hMPV/GFP2 |
| PreC23606M | 3.8 | <5 |
| PreC23611F | 2.1 | <5 |
| PreC23614F | 2.9 | <5 |
| Day28C23606M | 7 | 10 |
| Day28C23611F | 10 | 9 |
| Day28C23614F | 8 | 8.5 |
| Day35C23606M | NA | 10 |
| Day35C23611F | NA | 9.5 |
| Day35C23614F | NA | 10 |
| Day42C23606M | NA | 9.5 |
| Day42C23611F | NA | 10 |

TABLE 12
Linear Correlation between plaque reduction assay
and microneutralization assay using hMPV/GFP2

| Serum | Correlation (no trypsin) | Correlation (with trypsin) |
|---|---|---|
| Ferret 1 | 0.924 | 0.911 |
| Ferret 3 | 0.935 | 0.992 |
| Ferret 5 | 0.943 | 0.87 |
| Ferret 6 | 0.773 | 0.910 |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

TABLE 1
LEGEND FOR SEQUENCE LISTING

| | |
|---|---|
| SEQ ID NO: 1 | isolate NL/1/99 HMPV cDNA sequence (vaiant B1) |
| SEQ ID NO: 2 | isolate NL/1/00 HMPV cDNA sequence (variant A1) |
| SEQ ID NO: 3 | isolate NL/17/00 HMPV cDNA sequence (variant A2) |
| SEQ ID NO: 4 | isolate NL/1/94 HMPV cDNA sequence (variant B2) |
| SEQ ID NO: 5 | leader sequence of HMPV A1 |
| SEQ ID NO: 6 | leader sequence of HMPV B1 |
| SEQ ID NO: 7 | leader sequence of aMPV-C |
| SEQ ID NO: 8 | leader sequence of aMPV-A |
| SEQ ID NO: 9 | leader sequence of RSV |
| SEQ ID NO: 10 | leader sequence of PIV-3 |
| SEQ ID NO: 11 | trailer sequence of HMPV A1 |
| SEQ ID NO: 12 | trailer sequence of HMPV B1 |
| SEQ ID NO: 13 | trailer sequence of aMPV-C |
| SEQ ID NO: 14 | trailer sequence of aMPV-A |
| SEQ ID NO: 15 | trailer sequence of RSV |
| SEQ ID NO: 16 | trailer sequence of PIV-3 |
| SEQ ID NO: 17 | F protein sequence for HMPV isolate NL/1/00 |
| SEQ ID NO: 18 | F protein sequence for HMPV isolate NL/17/00 |
| SEQ ID NO: 19 | F protein sequence for HMPV isolate NL/1/99 |
| SEQ ID NO: 20 | F protein sequence for HMPV isolate NL/1/94 |
| SEQ ID NO: 21 | F-gene sequence for HMPV isolate NL/1/00 |
| SEQ ID NO: 22 | F-gene sequence for HMPV isolate NL/17/00 |
| SEQ ID NO: 23 | F-gene sequence for HMPV isolate NL/1/99 |
| SEQ ID NO: 24 | F-gene sequence for HMPV isolate NL/1/94 |
| SEQ ID NO: 25 | G protein sequence for HMPV isolate NL/1/00 |
| SEQ ID NO: 26 | G protein sequence for HMPV isolate NL/17/00 |
| SEQ ID NO: 27 | G protein sequence for HMPV isolate NL/1/99 |
| SEQ ID NO: 28 | G protein sequence for HMPV isolate NL/1/94 |
| SEQ ID NO: 29 | G-gene sequence for HMPV isolate NL/1/00 |
| SEQ ID NO: 30 | G-gene sequence for HMPV isolate NL/17/00 |
| SEQ ID NO: 31 | G-gene sequence for HMPV isolate NL/1/99 |
| SEQ ID NO: 32 | G-gene sequence for HMPV isolate NL/1/94 |
| SEQ ID NO: 33 | L protein sequence for HMPV isolate NL/1/00 |
| SEQ ID NO: 34 | L protein sequence for HMPV isolate NL/17/00 |
| SEQ ID NO: 35 | L protein sequence for HMPV isolate NL/1/99 |
| SEQ ID NO: 36 | L protein sequence for HMPV isolate NL/1/94 |
| SEQ ID NO: 37 | L-gene sequence for HMPV isolate NL/1/00 |
| SEQ ID NO: 38 | L-gene sequence for HMPV isolate NL/17/00 |
| SEQ ID NO: 39 | L-gene sequence for HMPV isolate NL/1/99 |
| SEQ ID NO: 40 | L-gene sequence for HMPV isolate NL/1/94 |
| SEQ ID NO: 41 | M2.1 protein sequence for HMPV isolate NL/1/00 |
| SEQ ID NO: 42 | M2.1 protein sequence for HMPV isolate NL/17/00 |
| SEQ ID NO: 43 | M2.1 protein sequence for HMPV isolate NL/1/99 |
| SEQ ID NO: 44 | M2.1 protein sequence for HMPV isolate NL/1/94 |
| SEQ ID NO: 45 | M2.1 gene sequence for HMPV isolate NL/1/00 |
| SEQ ID NO: 46 | M2.1 gene sequence for HMPV isolate NL/17/00 |
| SEQ ID NO: 47 | M2.1 gene sequence for HMPV isolate NL/1/99 |
| SEQ ID NO: 48 | M2.1 gene sequence for HMPV isolate NL/1/94 |
| SEQ ID NO: 49 | M2.2 protein sequence for HMPV isolate NL/1/00 |
| SEQ ID NO: 50 | M2.2 protein sequence for HMPV isolate NL/17/00 |
| SEQ ID NO: 51 | M2.2 protein sequence for HMPV isolate NL/1/99 |
| SEQ ID NO: 52 | M2.2 protein sequence for HMPV isolate NL/1/94 |
| SEQ ID NO: 53 | M2.2 gene sequence for HMPV isolate NL/1/00 |
| SEQ ID NO: 54 | M2.2 gene sequence for HMPV isolate NL/17/00 |
| SEQ ID NO: 55 | M2.2 gene sequence for HMPV isolate NL/1/99 |
| SEQ ID NO: 56 | M2.2 gene sequence for HMPV isolate NL/1/94 |
| SEQ ID NO: 57 | M2 gene sequence for HMPV isolate NL/1/00 |
| SEQ ID NO: 58 | M2 gene sequence for HMPV isolate NL/17/00 |
| SEQ ID NO: 59 | M2 gene sequence for HMPV isolate NL/1/99 |
| SEQ ID NO: 60 | M2 gene sequence for HMPV isolate NL/1/94 |
| SEQ ID NO: 61 | M protein sequence for HMPV isolate NL/1/00 |
| SEQ ID NO: 62 | M protein sequence for HMPV isolate NL/17/00 |
| SEQ ID NO: 63 | M protein sequence for HMPV isolate NL/1/99 |
| SEQ ID NO: 64 | M protein sequence for HMPV isolate NL/1/94 |
| SEQ ID NO: 65 | M gene sequence for HMPV isolate NL/1/00 |
| SEQ ID NO: 66 | M gene sequence for HMPV isolate NL/17/00 |
| SEQ ID NO: 67 | M gene sequence for HMPV isolate NL/1/99 |
| SEQ ID NO: 68 | M gene sequence for HMPV isolate NL/1/94 |
| SEQ ID NO: 69 | N protein sequence for HMPV isolate NL/1/00 |
| SEQ ID NO: 70 | N protein sequence for HMPV isolate NL/17/00 |
| SEQ ID NO: 71 | N protein sequence for HMPV isolate NL/1/99 |
| SEQ ID NO: 72 | N protein sequence for HMPV isolate NL/1/94 |
| SEQ ID NO: 73 | N gene sequence for HMPV isolate NL/1/00 |
| SEQ ID NO: 74 | N gene sequence for HMPV isolate NL/17/00 |
| SEQ ID NO: 75 | N gene sequence for HMPV isolate NL/1/99 |
| SEQ ID NO: 76 | N gene sequence for HMPV isolate NL/1/94 |
| SEQ ID NO: 77 | P protein sequence for HMPV isolate NL/1/00 |
| SEQ ID NO: 78 | P protein sequence for HMPV isolate NL/17/00 |
| SEQ ID NO: 79 | P protein sequence for HMPV isolate NL/1/99 |
| SEQ ID NO: 80 | P protein sequence for HMPV isolate NL/1/94 |
| SEQ ID NO: 81 | P gene sequence for HMPV isolate NL/1/00 |
| SEQ ID NO: 82 | P gene sequence for HMPV isolate NL/17/00 |
| SEQ ID NO: 83 | P gene sequence for HMPV isolate NL/1/99 |
| SEQ ID NO: 84 | P gene sequence for HMPV isolate NL/1/94 |
| SEQ ID NO: 85 | SH protein sequence for HMPV isolate NL/1/00 |
| SEQ ID NO: 86 | SH protein sequence for HMPV isolate NL/17/00 |
| SEQ ID NO: 87 | SH protein sequence for HMPV isolate NL/1/99 |
| SEQ ID NO: 88 | SH protein sequence for HMPV isolate NL/1/94 |
| SEQ ID NO: 89 | SH gene sequence for HMPV isolate NL/1/00 |
| SEQ ID NO: 90 | SH gene sequence for HMPV isolate NL/17/00 |
| SEQ ID NO: 91 | SH gene sequence for HMPV isolate NL/1/99 |
| SEQ ID NO: 92 | SH gene sequence for HMPV isolate NL/1/94 |
| SEQ ID NO: 93 | HMPV leader sequence |
| SEQ ID NO: 94 | HMPV trailer sequence |
| SEQ ID NO: 95 | APV leader sequence |
| SEQ ID NO: 96 | APV trailer sequence |
| SEQ ID NO: 97 | RSV A2 leader sequence |
| SEQ ID NO: 98 | RSV A2 trailer sequence |
| SEQ ID NO: 99 | BRSV leader sequence |
| SEQ ID NO: 100 | BRSV trailer sequence |
| SEQ ID NO: 101 | HPIV3 leader sequence |
| SEQ ID NO: 102 | HPIV3 trailer sequence |
| SEQ ID NO: 103 | BPIV3 leader sequence |
| SEQ ID NO: 104 | BPIV3 trailer sequence |
| SEQ ID NO: 105 | Primer hMPV, BspEI, +4741 |
| SEQ ID NO: 106 | Primer hMPV, BspEI, −4741 |
| SEQ ID NO: 107 | Primer hMPV, BspEI, +5444 |
| SEQ ID NO: 108 | Primer hMPV, Bsp EI, −5444 |
| SEQ ID NO: 109 | Primer hMPV, Nhe I, +4744 |
| SEQ ID NO: 110 | Primer hMPV, NheI, −4744 |
| SEQ ID NO: 111 | Primer hMPV, NheI, +5241 |
| SEQ ID NO: 112 | Primer hMPV, Nhe I, −5241 |
| SEQ ID NO: 113 | Primer hMPV, SwaI, +5311 |
| SEQ ID NO: 114 | Primer hMPV, SwaI, −5311 |
| SEQ ID NO: 115 | Primer hMPV, SwaI, +5453 |
| SEQ ID NO: 116 | Primer hMPV, SwaI, −5453 |
| SEQ ID NO: 117 | Primer hMPV, SacII +5472 |
| SEQ ID NO: 118 | Primer hMPV, SacII −5472 |
| SEQ ID NO: 119 | Primer hMPV, SacII +6026 |
| SEQ ID NO: 120 | Primer hMPV, SacII −6026 |
| SEQ ID NO: 121 | CAT-HMPV minireplicon construct |
| SEQ ID NO: 122 | CAT-HMPV minireplicon construct nucleotide |
| | sequence |
| SEQ ID NO: 123 | Primer hMPV mutagenesis RF1410 |
| SEQ ID NO: 124 | Primer hMPV mutagenesis RF1502 |
| SEQ ID NO: 125 | Primer hMPV mutagenesis RF1503 |
| SEQ ID NO: 126 | Primer hMPV mutagenesis RF1504 |
| SEQ ID NO: 127 | Primer hMPV mutagenesis RF1505 |
| SEQ ID NO: 128 | Primer hMPV mutagenesis RF1430 |
| SEQ ID NO: 129 | Primer hMPV mutagenesis RF1411 |
| SEQ ID NO: 130 | Primer hMPV mutagenesis RF1506 |
| SEQ ID NO: 131 | Primer hMPV mutagenesis RF1412 |
| SEQ ID NO: 132 | Primer hMPV mutagenesis RF1413 |
| SEQ ID NO: 133 | Primer hMPV mutagenesis RF1414 |
| SEQ ID NO: 134 | Primer hMPV mutagenesis RF1508 |
| SEQ ID NO: 135 | Primer hMPV mutagenesis RF1416 |
| SEQ ID NO: 136 | Primer hMPV mutagenesis RF1417 |
| SEQ ID NO: 137 | Primer hMPV mutagenesis RF1509 |
| SEQ ID NO: 138 | Primer hMPV mutagenesis RF1510 |
| SEQ ID NO: 139 | Primer hMPV mutagenesis RF1418 |
| SEQ ID NO: 140 | Primer hMPV mutagenesis RF1422 |
| SEQ ID NO: 141 | Primer cpRSV mutagenesis RF1415 |
| SEQ ID NO: 142 | Primer cpRSV mutagenesis RF1419 |
| SEQ ID NO: 143 | Primer cpRSV mutagenesis RF1420 |
| SEQ ID NO: 144 | Primer cpRSV mutagenesis RF1421 |
| SEQ ID NO: 145 | Primer NL/1/99 sequencing RF665 |
| SEQ ID NO: 146 | Primer NL/1/99 sequencing BF30 |
| SEQ ID NO: 147 | Primer NL/1/99 sequencing BF29 |
| SEQ ID NO: 148 | Primer NL/1/99 sequencing RF524 |
| SEQ ID NO: 149 | Primer NL/1/99 sequencing RF1515 |
| SEQ ID NO: 150 | Primer NL/1/99 sequencing RF1516 |
| SEQ ID NO: 151 | Primer NL/1/99 sequencing RF846 |
| SEQ ID NO: 152 | Primer NL/1/99 sequencing RF847 |
| SEQ ID NO: 153 | Primer NL/1/99 sequencing RF848 |
| SEQ ID NO: 154 | Primer NL/1/99 sequencing RF849 |
| SEQ ID NO: 155 | Primer NL/1/99 sequencing RF850 |
| SEQ ID NO: 156 | Primer NL/1/99 sequencing RF851 |
| SEQ ID NO: 157 | Primer NL/1/99 sequencing RF852 |
| SEQ ID NO: 158 | Primer NL/1/99 sequencing RF853 |
| SEQ ID NO: 159 | Primer NL/1/99 sequencing RF1517 |
| SEQ ID NO: 160 | Primer NL/1/99 sequencing RF1518 |
| SEQ ID NO: 161 | Primer NL/1/99 sequencing BF33 |
| SEQ ID NO: 162 | Primer NL/1/99 sequencing BF25 |
| SEQ ID NO: 163 | Primer NL/1/99 sequencing RF856 |
| SEQ ID NO: 164 | Primer NL/1/99 sequencing RF857 |
| SEQ ID NO: 165 | Primer NL/1/99 sequencing RF858 |
| SEQ ID NO: 166 | Primer NL/1/99 sequencing RF859 |
| SEQ ID NO: 167 | Primer NL/1/99 sequencing RF860 |
| SEQ ID NO: 168 | Primer NL/1/99 sequencing RF861 |
| SEQ ID NO: 169 | Primer NL/1/99 sequencing RF1519 |
| SEQ ID NO: 170 | Primer NL/1/99 sequencing RF1520 |
| SEQ ID NO: 171 | Primer NL/1/99 sequencing RF1521 |
| SEQ ID NO: 172 | Primer NL/1/99 sequencing RF1522 |
| SEQ ID NO: 173 | Primer NL/1/99 sequencing RF1049 |
| SEQ ID NO: 174 | Primer NL/1/99 sequencing RF1050 |
| SEQ ID NO: 175 | Primer NL/1/99 sequencing RF1404 |
| SEQ ID NO: 176 | Primer NL/1/99 sequencing RF1405 |
| SEQ ID NO: 177 | Primer NL/1/99 sequencing RF862 |
| SEQ ID NO: 178 | Primer NL/1/99 sequencing RF863 |
| SEQ ID NO: 179 | Primer NL/1/99 sequencing RF864 |
| SEQ ID NO: 180 | Primer NL/1/99 sequencing RF865 |
| SEQ ID NO: 181 | Primer NL/1/99 sequencing RF866 |
| SEQ ID NO: 182 | Primer NL/1/99 sequencing RF867 |
| SEQ ID NO: 183 | Primer NL/1/99 sequencing RF868 |
| SEQ ID NO: 184 | Primer NL/1/99 sequencing RF869 |
| SEQ ID NO: 185 | Primer NL/1/99 sequencing RF870 |
| SEQ ID NO: 186 | Primer NL/1/99 sequencing RF871 |
| SEQ ID NO: 187 | Primer NL/1/99 sequencing RF872 |
| SEQ ID NO: 188 | Primer NL/1/99 sequencing RF1523 |
| SEQ ID NO: 189 | Primer NL/1/99 sequencing RF1524 |
| SEQ ID NO: 190 | Primer NL/1/99 sequencing RF875 |
| SEQ ID NO: 191 | Primer NL/1/99 sequencing RF876 |
| SEQ ID NO: 192 | Primer NL/1/99 sequencing BF21 |
| SEQ ID NO: 193 | Primer NL/1/99 sequencing RF934 |
| SEQ ID NO: 194 | Primer NL/1/99 sequencing RF1525 |
| SEQ ID NO: 195 | Primer NL/1/99 sequencing RF1526 |

1. An isolated mammalian metapneumovirus, wherein the isolated mammalian metapneumovirus comprises a genetic modification resulting in an amino acid substitution, deletion, or insertion at one or more amino acid positions selected from the group consisting of: position 66 in the P protein; positions 9, 38, 52, and 132 in the M protein; positions 93, 109, 280, 471, 532, and 538 in the F protein; position 187 in the M2 protein; positions 139 and 164 in the G protein; and positions 235, 323, and 1453 in the L protein, with the proviso that the modification at position 101 in the F protein is not a substitution to Proline and that the modification at position 93 in the F protein is not a substitution to Lysine.

2. The isolated mammalian metapneumovirus of claim 1, wherein the genetic modification results in an amino acid substitution, deletion, or insertion at one or more amino acid positions selected from the group consisting of: position 66 in the P protein; position 132 in the M protein; positions 101, 280, and 471 in the F protein; position 187 in the M2 protein; position 139 in the G protein; and positions 235, 323, and 1453 in the L protein, with the proviso that modification at position 101 in the F protein is not a substitution to Proline.

3. The isolated mammalian metapneumovirus of claim 1, wherein the genetic modification results in an amino acid substitution, deletion, or insertion at one or more amino acid positions selected from the group consisting of: position 132 in the M protein; positions 101, 280, and 471 in the F protein; position 187 in the M2 protein; position 139 in the G protein; and position 1453 in the L protein, with the proviso that modification at position 101 in the F protein is not a substitution to Proline.

4. The isolated mammalian metapneumovirus of claim 1, wherein the genetic modification results in an amino acid substitution, deletion, or insertion at one or more amino acid positions selected from the group consisting of: positions 235 and 323 in the L protein.

5. An isolated mammalian metapneumovirus, wherein the isolated mammalian metapneumovirus comprises a genetic modification at one or more of the nucleotide positions selected from the group consisting of: position 197 in the P open reading frame; position 9, 113, 155, 336, 394, and 436 in the M open reading frame; positions 277, 301, 325, 839, 1412, 1594, and 1612 in the F open reading frame; position 560 in the M2 open reading frame; position 415 and 491 in the G open reading frame; and positions 703, 967, and 4357 in the L open reading frame.

6. An isolated mammalian metapneumovirus, wherein the isolated mammalian metapneumovirus comprises a genetic modification resulting in one or more amino acid changes selected from the group consisting of:
position 66 in the P protein is altered to Val;
position 9 in the M protein is altered to His;
position 38 in the M protein is altered to Ser;
position 52 in the M protein is altered to Pro;
position 132 in the M protein is altered to Pro;
position 93 in the F protein is altered to Lys;
position 109 in the F protein is altered to Ser;
position 280 in the F protein is altered to Gly;
position 471 in the F protein is altered to Arg;
position 532 in the F protein is altered to Tyr;
position 538 in the F protein is altered to Tyr;
position 187 in the M2 protein is altered to Ile;
position 139 in the G protein is altered to Pro;
position 164 in the G protein is altered to Pro;
position 235 in the L protein is altered to Arg;
position 323 in the L protein is altered to Asp; and
position 1453 in the L protein is altered to Leu.

7. The isolated mammalian metapneumovirus of claim 1, wherein the isolated mammalian metapneumovirus comprises at least two, at least three, at least four, at least five, at least six, at least seven or at least eight of the specified genetic modifications.

8. The isolated mammalian metapneumovirus of claim 4, wherein the isolated mammalian metapneumovirus comprises genetic modifications resulting in amino acid substitution, deletion, or insertion at amino acid positions 235 and 323 in the L protein.

9. A recombinant mammalian metapneumovirus, wherein the recombinant mammalian metapneumovirus comprises two or more genetic modifications, wherein the genetic modification is an amino acid substitution, deletion, or insertion amino acid position 456 of the L gene; position 1094 of the L gene; or position 1246 of the L gene; or a nucleotide substitution, deletion, or insertion at the gene start sequence of the M2 gene.

10. A recombinant mammalian metapneumovirus, wherein the gene start sequence of the M2 gene of MPV is altered; Phe at amino acid position 456 of the L gene is mutated to Leu; and Met at amino acid position 1094 of the L gene is mutated to Val.

11. The mammalian metapneumovirus of claim 1, wherein the virus is attenuated.

12. The mammalian metapneumovirus of claim 1, wherein at least one of the genetic alterations consists of 2 or 3 nucleotide substitutions per codon.

13. The mammalian metapneumovirus of claim 1, wherein the virus is temperature-sensitive.

14. The mammalian metapneumovirus of claim 1, wherein the virus is a human metapneumovirus.

15. The human metapneumovirus of claim 14, wherein the human metapneumovirus is variant A1, A2, B1, or B2.

16. The human metapneumovirus of claim 14, wherein the human metapneumovirus is HMPV strain NL/1/99, NL/1 7/00, NL/1/00, or NL/1/94.

17. A method of stimulating the immune response against mammalian metapneumovirus in a mammal, said method comprising administering to the mammal the mammalian metapneumovirus of claim 1.

18.-22. (canceled)

23. An immunogenic composition comprising the metapneumovirus of claim 1, 2, 3, 4, 5, 6, 8, or 9 and a pharmaceutically acceptable excipient.

24. (canceled)

25. (canceled)

26. A method of producing a mammalian metapneumovirus comprising: a) introducing a recombinant nucleic acid comprising a cDNA encoding the mammalian metapneumovirus of claim 1 operatively linked to a promoter for a DNA-directed RNA polymerase into a host cell, wherein the host cell expresses (i) the N, P, and L proteins of a mammalian metapneumovirus and (ii) the DNA-directed RNA polymerase; and b) isolating the virus produced by the host cell.

27.-49. (canceled)
